SEQUENCE LISTING (579 sequences)

SEQ ID NO: 1 - DNA, 21 bp, Artificial Sequence
  Synthetic oligonucleotide
ctggaaggtgtctaccatcga

SEQ ID NO: 2 - DNA, 19 bp, Artificial Sequence
  Synthetic oligonucleotide
aaagaagcccaggctgaca

SEQ ID NO: 3 - DNA, 21 bp, Artificial Sequence
  Synthetic oligonucleotide
taaaccctgacccatctcaga

SEQ ID NO: 4 - DNA, 21 bp, Artificial Sequence
  Synthetic oligonucleotide
tgagactcatcagactggtga

SEQ ID NO: 5 - DNA, 21 bp, Artificial Sequence
  Synthetic oligonucleotide
aatgactgggttgtcgatgga

SEQ ID NO: 6 - DNA, 20 bp, Artificial Sequence
  Synthetic oligonucleotide
acagcggaatcttctgctga

SEQ ID NO: 7 - DNA, 20 bp, Artificial Sequence
  Synthetic oligonucleotide
tcctgcaagtgcaaagagtg

SEQ ID NO: 8 - DNA, 20 bp, Artificial Sequence
  Synthetic oligonucleotide
ggaatgtagcaaaggggtca

SEQ ID NO: 9 - DNA, 20 bp, Artificial Sequence
  Synthetic oligonucleotide
aggtggaggctttgtcccta

SEQ ID NO: 10 - DNA, 20 bp, Artificial Sequence
  Synthetic oligonucleotide
tataccatctggccccacca

SEQ ID NO: 11 - DNA, 19 bp, Artificial Sequence
  Synthetic oligonucleotide
gaaggtgaaggtcggagtc

SEQ ID NO: 12 - DNA, 20 bp, Artificial Sequence
  Synthetic oligonucleotide
gaagatggtgatgggatttc

SEQ ID NO: 13 - DNA, 60 bp, Artificial Sequence
  Synthetic oligonucleotide
gtaactgtgctgaatgctttagatgaggaaatgatccccaagtggtgaatgacacgccta

SEQ ID NO: 14 - DNA, 60 bp, Artificial Sequence
  Synthetic oligonucleotide
ggcgagattgaagggcttttgttattgttgttggatatttttgtttcccataaaagcaca

SEQ ID NO: 15 - DNA, 60 bp, Artificial Sequence
  Synthetic oligonucleotide
tttgaccgcagcatgcacaagctccaaagtggaattggccggctgattctgaaggaagaa

SEQ ID NO: 16 - DNA, 60 bp, Artificial Sequence
  Synthetic oligonucleotide
tttctggaagagattgcatctgaggaaattcaggaaggatctttgtagattggggggaga

SEQ ID NO: 17 - DNA, 60 bp, Artificial Sequence
  Synthetic oligonucleotide
tgtgttctcatgtaggatgtcagccctccctgcaacttctctttttggccaatgtctttt

SEQ ID NO: 18 - DNA, 60 bp, Artificial Sequence
  Synthetic oligonucleotide
aagccattcataaaagaagcgtcaatggccaaatactatgcatcagagattgcaggacaa

SEQ ID NO: 19 - DNA, 60 bp, Artificial Sequence
  Synthetic oligonucleotide
caacatccagttgaacaccattgcaaagcatatcgatgcagaatactgacgtctatagga

SEQ ID NO: 20 - DNA, 60 bp, Artificial Sequence
  Synthetic oligonucleotide
aattcagaaattgggttttggttcagtgattctcaagaaaaagatctcttgcccattaag

SEQ ID NO: 21 - DNA, 60 bp, Artificial Sequence
  Synthetic oligonucleotide
atcatgaagtgtgatattgacatcaggaaggacctctatgctaacaatgtcctatcaggg

SEQ ID NO: 22 - DNA, 60 bp, Artificial Sequence
  Synthetic oligonucleotide
accgcaaatgcttctaaaacactttcccgctcctctctgtctctagcacacaactgtgaa

SEQ ID NO: 23 - DNA, 60 bp, Artificial Sequence
  Synthetic oligonucleotide
acctctgagcagtgatatagcataataaagccccgggcattattattattatttcttttg

SEQ ID NO: 24 - DNA, 60 bp, Artificial Sequence
  Synthetic oligonucleotide
tatgggggtagatagaaaaggagttgaatcatcagagtaaactgccagttgcaaatttga

SEQ ID NO: 25 - DNA, 60 bp, Artificial Sequence
  Synthetic oligonucleotide
ctttataagtattggtttgggtgttccttccaagaaggactatagttagtaataaatgcc

SEQ ID NO: 26 - DNA, 60 bp, Artificial Sequence
  Synthetic oligonucleotide
gaagaggagaggggatggggcgacagatcctatcatcaactgtccagtggactggacctt

SEQ ID NO: 27 - DNA, 60 bp, Artificial Sequence
  Synthetic oligonucleotide
caccaattatggctacaatgacattgtcaccatcccagctggtgccactaatattgacgt

SEQ ID NO: 28 - DNA, 60 bp, Artificial Sequence
  Synthetic oligonucleotide
atcatgtcattgatgaactgccaaagtcaggggaggagggcagagactttgtgtttacat

SEQ ID NO: 29 - DNA, 60 bp, Artificial Sequence
  Synthetic oligonucleotide
actacaaggacatggctcacaaaaggttaatggatggttacctagaggattacctagccc

SEQ ID NO: 30 - DNA, 60 bp, Artificial Sequence
  Synthetic oligonucleotide
ctcctcaatctggtttatgaaacaactgacaaacacctttctcctgatggccagtatgtc

SEQ ID NO: 31 - DNA, 60 bp, Artificial Sequence
  Synthetic oligonucleotide
ttttttctctttttggtggtggttaaaagggaacacaaaacatttaaataaaactttcca

SEQ ID NO: 32 - DNA, 60 bp, Artificial Sequence
  Synthetic oligonucleotide
tcctcggaagacactctgaccgtggtcactgcggaccattcccacgtcttcacatttggt

SEQ ID NO: 33 - DNA, 60 bp, Artificial Sequence
  Synthetic oligonucleotide
gctacaaggtggtgggcggtgaacgagagaatgtctccatggtggactatgctcacaaca

SEQ ID NO: 34 - DNA, 60 bp, Artificial Sequence
  Synthetic oligonucleotide
acctggaagagcttcaaaccgagatacaagcactcccacttcatctggaaccgcacggaa

SEQ ID NO: 35 - DNA, 60 bp, Artificial Sequence
  Synthetic oligonucleotide
tcttagcttttagcactattggtaatttcagagtaggcccaaaggtgatatgactcccat

SEQ ID NO: 36 - DNA, 60 bp, Artificial Sequence
  Synthetic oligonucleotide
ggcccaaagaaagagctacctggaccttttgttttctgtttgacaacatgtttaataaat

SEQ ID NO: 37 - DNA, 60 bp, Artificial Sequence
  Synthetic oligonucleotide
agagctacctggaccttttgttttctgtctgacaacatgtttaataaataaaaatgtcac

SEQ ID NO: 38 - DNA, 60 bp, Artificial Sequence
  Synthetic oligonucleotide
gtgtcatattaggttaataaggctgctgtgttttaaagggcatttttatttgggttttgg

SEQ ID NO: 39 - DNA, 60 bp, Artificial Sequence
  Synthetic oligonucleotide
ctcgtaacagcgaacggtcagtcaagggatcataagtttttactgccagtattgagaaat

SEQ ID NO: 40 - DNA, 60 bp, Artificial Sequence
  Synthetic oligonucleotide
acttttattctttcaaagaaaatagactgccattttccatcaagattagagacaccagcc

SEQ ID NO: 41 - DNA, 60 bp, Artificial Sequence
  Synthetic oligonucleotide
atcagcattcattgacacatagctctaatgacatatgtatgaaaaaccatacactggatg

SEQ ID NO: 42 - DNA, 60 bp, Artificial Sequence
  Synthetic oligonucleotide
tgttggaataccggcggtgatctgtcttttataaactcacctgatttaaaggaaagatga

SEQ ID NO: 43 - DNA, 60 bp, Artificial Sequence
  Synthetic oligonucleotide
agcacccgtaaatggactttggtctcaatgctttgactctttgccgtggttttggatgtt

SEQ ID NO: 44 - DNA, 60 bp, Artificial Sequence
  Synthetic oligonucleotide
aattaaaacgcctacagctgcctcctagaatatagactgtctgtattattattcacctat

SEQ ID NO: 45 - DNA, 60 bp, Artificial Sequence
  Synthetic oligonucleotide
ctttctgctgtcaccttcgtcttgtcagaatgaatatagacactgtatctaagtgggacc

SEQ ID NO: 46 - DNA, 60 bp, Artificial Sequence
  Synthetic oligonucleotide
ttggtttcctctagggtgatattcgtcattactctgtctcttcaatccatccagctaaat

SEQ ID NO: 47 - DNA, 60 bp, Artificial Sequence
  Synthetic oligonucleotide
ctgtccatctgtgcataaggagaggaaagttccagggtgtgtatgtttcaggggcttcac

SEQ ID NO: 48 - DNA, 60 bp, Artificial Sequence
  Synthetic oligonucleotide
aatttgggtcaatacatccttttgtctcccaagggaagagaatgggcagcaggtatgtgt

SEQ ID NO: 49 - DNA, 60 bp, Artificial Sequence
  Synthetic oligonucleotide
tttggtcagacaagagaaggagggcatattgtctatgaccaacttcctactcccagttca

SEQ ID NO: 50 - DNA, 60 bp, Artificial Sequence
  Synthetic oligonucleotide
tcaggttccagtagttcattctaatgcctagattcttttgtggttgttgctggcccaatg

SEQ ID NO: 51 - DNA, 60 bp, Artificial Sequence
  Synthetic oligonucleotide
ctagtcacatcccctgccagatggagttcttcttttgtgagagacactgtaaacaacaca

SEQ ID NO: 52 - DNA, 60 bp, Artificial Sequence
  Synthetic oligonucleotide
aagactggcaaacagatggcaagggatgcccctctttttcataaaactctccaaggttca

SEQ ID NO: 53 - DNA, 60 bp, Artificial Sequence
  Synthetic oligonucleotide
tggtgcatgaattctcaagtactgtatttcactgtgttggtgtgtctgatggaaatttcg

SEQ ID NO: 54 - DNA, 60 bp, Artificial Sequence
  Synthetic oligonucleotide
gagcacttaaagtccagtgttggctgttagtgtatttgatattctgcctgtctcctcatg

SEQ ID NO: 55 - DNA, 60 bp, Artificial Sequence
  Synthetic oligonucleotide
agaacgaataccctatttaggttttaaacagattaacctttggactatgtttcaagctgc

SEQ ID NO: 56 - DNA, 60 bp, Artificial Sequence
  Synthetic oligonucleotide
atgtagagatccagtgttaagagttccatttgcttcaattaattatttaccttcctgtgg

SEQ ID NO: 57 - DNA, 60 bp, Artificial Sequence
  Synthetic oligonucleotide
atcagggcatatacaaaagggtttgttaaaactcgatgttaactttacaactttctgacc

SEQ ID NO: 58 - DNA, 60 bp, Artificial Sequence
  Synthetic oligonucleotide
atcacaaatcccctgcaagctattcaaatggtgatggatacgcttggcattccttattag

SEQ ID NO: 59 - DNA, 60 bp, Artificial Sequence
  Synthetic oligonucleotide
tctttggaagataacagtgatgtcacagggttggctatgtttattctgaatcgcctactt

SEQ ID NO: 60 - DNA, 60 bp, Artificial Sequence
  Synthetic oligonucleotide
aggccgagtaagagagattatgacatttcttgtaaatgatgttctgaaacaccaagctat

SEQ ID NO: 61 - DNA, 60 bp, Artificial Sequence
  Synthetic oligonucleotide
atcacaaatcccctgcaagccattcaaatggtgatggatacacttggcattccttattag

SEQ ID NO: 62 - DNA, 60 bp, Artificial Sequence
  Synthetic oligonucleotide
gtggggggcagctgcggtggggagctataaaatgacaattaaaagatacactagtctttt

SEQ ID NO: 63 - DNA, 60 bp, Artificial Sequence
  Synthetic oligonucleotide
attgttgtgataatttgtaattgtgacttgttctccccggctggcagcgtagtggggctg

SEQ ID NO: 64 - DNA, 60 bp, Artificial Sequence
  Synthetic oligonucleotide
tccctggaggatgcctgaattctacaaccggttcaagggccgcaatgacctgatggagta

SEQ ID NO: 65 - DNA, 60 bp, Artificial Sequence
  Synthetic oligonucleotide
tgtgaaggtgaccaacgtcaaggatggcaccacccaccagacctccttggagctcttcat

SEQ ID NO: 66 - DNA, 60 bp, Artificial Sequence
  Synthetic oligonucleotide
aataattgccaggagtacagtgctcttgttgatcttgtattcagtcaggttaaaacaatg

SEQ ID NO: 67 - DNA, 60 bp, Artificial Sequence
  Synthetic oligonucleotide
aactgtcatacgtatgggacctacacttaatctctatgctttacactagcttctgcattt

SEQ ID NO: 68 - DNA, 60 bp, Artificial Sequence
  Synthetic oligonucleotide
aataattgccaggagtacagtgctcttgttgatcttgtattcagtcaggttaaaacaacg

SEQ ID NO: 69 - DNA, 60 bp, Artificial Sequence
  Synthetic oligonucleotide
tgggtggggggttgtcatgggggaactgccctttaaattttaagtgacactacagaaaaa

SEQ ID NO: 70 - DNA, 60 bp, Artificial Sequence
  Synthetic oligonucleotide
tgaagcagagaaacattcacacacaaaaagcaacatagtcatgtgggtccagatggcctc

SEQ ID NO: 71 - DNA, 60 bp, Artificial Sequence
  Synthetic oligonucleotide
atgtgggtccagatggcctcagtcctagatgttggcaccctttgctgtgtctcctcagag

SEQ ID NO: 72 - DNA, 60 bp, Artificial Sequence
  Synthetic oligonucleotide
tatgctcagatattcatcgtaagtctcccttcacctgttacagagtttcagatcggtcac

SEQ ID NO: 73 - DNA, 60 bp, Artificial Sequence
  Synthetic oligonucleotide
tttgtcctaggtgtaccctttcctcatctctattaaattgtaaacaggactactgcatgt

SEQ ID NO: 74 - DNA, 60 bp, Artificial Sequence
  Synthetic oligonucleotide
gtagaggaaagggtgtgtgaacatggctaacaatctcaaatacccaaattgtgatagcat

SEQ ID NO: 75 - DNA, 60 bp, Artificial Sequence
  Synthetic oligonucleotide
ttgatggatgctacttctatttgtggggaaccttcaaacaccatccaaaggacaacctta

SEQ ID NO: 76 - DNA, 60 bp, Artificial Sequence
  Synthetic oligonucleotide
gcacggtttgtgagagcccagtcattgtgctgtttttaatttttcacatttttacaaata

SEQ ID NO: 77 - DNA, 60 bp, Artificial Sequence
  Synthetic oligonucleotide
ctgcgttaaaggctcgattttgtatctgcaggcagacacggatctgagaatctttattga

SEQ ID NO: 78 - DNA, 60 bp, Artificial Sequence
  Synthetic oligonucleotide
tttggctgtgtctaaactgcatcaccgcgttgtaaaatatagctgtacaaatatcagaat

SEQ ID NO: 79 - DNA, 60 bp, Artificial Sequence
  Synthetic oligonucleotide
atcagcgtcctatacattgaaggtgtgcatatatgttgaatgacattttagggacatggt

SEQ ID NO: 80 - DNA, 60 bp, Artificial Sequence
  Synthetic oligonucleotide
tcctatacatcgaaggtgtgcatatatgttgaatgacattttagggacatggtgttttta

SEQ ID NO: 81 - DNA, 60 bp, Artificial Sequence
  Synthetic oligonucleotide
ctaaacaggctcattacaaatggttaccttgttatttaacccatttgtctctacttttcc

SEQ ID NO: 82 - DNA, 60 bp, Artificial Sequence
  Synthetic oligonucleotide
cacagtgtctgttcttggggagcttgcagcagaaatgaatggggtttttgacactacatt

SEQ ID NO: 83 - DNA, 60 bp, Artificial Sequence
  Synthetic oligonucleotide
cgccccagcagtgtcctcagaacactctactcgcacctcccggtgatccatgaactctga

SEQ ID NO: 84 - DNA, 60 bp, Artificial Sequence
  Synthetic oligonucleotide
aagaccaccgcaggatgggcagcagagctctgacctaagaagctggacttttgggcagtc

SEQ ID NO: 85 - DNA, 60 bp, Artificial Sequence
  Synthetic oligonucleotide
tgtgttttctctggagatagaatgtaaaccatattaaaaggaaaaagtttcagacaagca

SEQ ID NO: 86 - DNA, 60 bp, Artificial Sequence
  Synthetic oligonucleotide
gtaaaccaaaaacttttaaatttcttcaggttttctaacatgcttaccactgggctactg

SEQ ID NO: 87 - DNA, 60 bp, Artificial Sequence
  Synthetic oligonucleotide
tcgctagcacaaaatattgtcgctaatagtcatttctgttttcccattgtaaatgctgtt

SEQ ID NO: 88 - DNA, 60 bp, Artificial Sequence
  Synthetic oligonucleotide
acacacgcttattgccgtggcaacagcggtagtgctagggggattaatttttatagcatt

SEQ ID NO: 89 - DNA, 60 bp, Artificial Sequence
  Synthetic oligonucleotide
tcaacttaagcataaagggctctgaacttttccactttagagtgaccgtcatttcaggag

SEQ ID NO: 90 - DNA, 60 bp, Artificial Sequence
  Synthetic oligonucleotide
atgccaggacctcggcaccttcactgagaacatggttgtctttgggtgccccaactgacc

SEQ ID NO: 91 - DNA, 60 bp, Artificial Sequence
  Synthetic oligonucleotide
gtgccgctgtgctgaggagaattgcttcatacaaaagtcggatgacaaggtcaccctgga

SEQ ID NO: 92 - DNA, 60 bp, Artificial Sequence
  Synthetic oligonucleotide
atgccaagacgaagagaaccagaaacaatgccaggacctcggcgccttcaccgagagcat

SEQ ID NO: 93 - DNA, 60 bp, Artificial Sequence
  Synthetic oligonucleotide
accgtgccttatgcctgtgtgtgatcagtttctggcacacagatgcctcaataaagattt

SEQ ID NO: 94 - DNA, 60 bp, Artificial Sequence
  Synthetic oligonucleotide
cctcttgcaaatcaatacagatcagtttagcaaatctactgtcaatttggcagtgatatt

SEQ ID NO: 95 - DNA, 60 bp, Artificial Sequence
  Synthetic oligonucleotide
ttctcaaaagaaggaggaaaaggtgtcttgctggcttgcctcttgcaattcaatacagat

SEQ ID NO: 96 - DNA, 60 bp, Artificial Sequence
  Synthetic oligonucleotide
gctggatgcccttttgaaggatctgtacgagaaaaacaaaaaggaaatgaatattcaaca

SEQ ID NO: 97 - DNA, 60 bp, Artificial Sequence
  Synthetic oligonucleotide
gtagtacttagcctacctagaccagcaagcattcatttttagctcgctcattttttacca

SEQ ID NO: 98 - DNA, 60 bp, Artificial Sequence
  Synthetic oligonucleotide
cttctgcagtgccttcactacactgccttacataaaccaaatcacaataaagttcatatt

SEQ ID NO: 99 - DNA, 60 bp, Artificial Sequence
  Synthetic oligonucleotide
gtggtgaatatcagtagtttgcagtgtttaagggcttttgaaaactgcagtgaagatctg

SEQ ID NO: 100 - DNA, 60 bp, Artificial Sequence
  Synthetic oligonucleotide
caggtcatgaggtttcattatcggcagtaactcgttgcacatgtttctagtggcaaaaaa

SEQ ID NO: 101 - DNA, 60 bp, Artificial Sequence
  Synthetic oligonucleotide
ttggtccatgacaaagttgtgcaaaactggtaaacgtctgcttcggagcttgctgcttaa

SEQ ID NO: 102 - DNA, 60 bp, Artificial Sequence
  Synthetic oligonucleotide
tgtgtagttaagcaccaaacagcagagagaacttagacactaccacaccaagccttgtga

SEQ ID NO: 103 - DNA, 60 bp, Artificial Sequence
  Synthetic oligonucleotide
ttgattttgtacatagtcctctggtctatctcatgaaacctcttctcagaccagttttct

SEQ ID NO: 104 - DNA, 60 bp, Artificial Sequence
  Synthetic oligonucleotide
agtcctctggtctatctcatgaaacctcttctcagaccaattttctaaatatatattgag

SEQ ID NO: 105 - DNA, 60 bp, Artificial Sequence
  Synthetic oligonucleotide
ggcagaggacgggaggagaccagtcccccacccagccgtaccagaaataaaggcttctgt

SEQ ID NO: 106 - DNA, 60 bp, Artificial Sequence
  Synthetic oligonucleotide
catgtcccgcagccgctgcaacacgctgtcctcccccaaccagtaccagtgacccagtgg

SEQ ID NO: 107 - DNA, 60 bp, Artificial Sequence
  Synthetic oligonucleotide
gacaacctgactatcaccatgcaccgcctgcagctgtcggacactggcacctacacctgc

SEQ ID NO: 108 - DNA, 60 bp, Artificial Sequence
  Synthetic oligonucleotide
atcagcctccccaaggttctgtcctgttccgagcaacttttctaattataaacatcacag

SEQ ID NO: 109 - DNA, 60 bp, Artificial Sequence
  Synthetic oligonucleotide
agaatgcccactgcctgtaacagccacctggagaacttcataaagatgtctcacagccct

SEQ ID NO: 110 - DNA, 60 bp, Artificial Sequence
  Synthetic oligonucleotide
cccatgtcaaaaacttggatgaaaatggcttggatttgctctcgaaaatgttaatctatg

SEQ ID NO: 111 - DNA, 60 bp, Artificial Sequence
  Synthetic oligonucleotide
tatcaaacttaagctgtacttcatcttctaatttcaaaagtataacttaaaaatgtaaat

SEQ ID NO: 112 - DNA, 60 bp, Artificial Sequence
  Synthetic oligonucleotide
atccaccaaggcatccgctgaagaccaacccatcacctcagttgttttttatttttctaa

SEQ ID NO: 113 - DNA, 60 bp, Artificial Sequence
  Synthetic oligonucleotide
ccgctgaagactaacccatcacctcagttgttttttatttttctaataaagtcatgtctc

SEQ ID NO: 114 - DNA, 60 bp, Artificial Sequence
  Synthetic oligonucleotide
ggtaatgataacttggtcaatgtgtggcctagtgctcctggagagggtggctgggttcct

SEQ ID NO: 115 - DNA, 60 bp, Artificial Sequence
  Synthetic oligonucleotide
ctgagtgcaaaactcagtagactcctctttgtcacttctctggagatccagcattcctta

SEQ ID NO: 116 - DNA, 60 bp, Artificial Sequence
  Synthetic oligonucleotide
ggctttactttgccactagagttgaaatataagggaacaggaaatgaatgcattgtggta

SEQ ID NO: 117 - DNA, 60 bp, Artificial Sequence
  Synthetic oligonucleotide
acctgaagagcttcaagctgaaaggaatttccacactgtcccctacatggtcggaattaa

SEQ ID NO: 118 - DNA, 60 bp, Artificial Sequence
  Synthetic oligonucleotide
gagcaatgaagtcatccactcctgcatctggttggtctttattgagcacctactatatgc

SEQ ID NO: 119 - DNA, 60 bp, Artificial Sequence
  Synthetic oligonucleotide
aacctgggcaagcatgagaagttctcggaggtgcttaagcggctgcgacttcagaagcga

SEQ ID NO: 120 - DNA, 60 bp, Artificial Sequence
  Synthetic oligonucleotide
agaaatgaagcccggcccacacccgacaccagccctgctgcttcctaacttattgcctgg

SEQ ID NO: 121 - DNA, 60 bp, Artificial Sequence
  Synthetic oligonucleotide
ctaacttattgcctgggcagtgcccaccatgcacccctgatgttcgccgcctggcgagcc

SEQ ID NO: 122 - DNA, 60 bp, Artificial Sequence
  Synthetic oligonucleotide
gtaagaaactgtctgatatgaatcacaacatggatgaatgtagtattttcctgaagtgtg

SEQ ID NO: 123 - DNA, 60 bp, Artificial Sequence
  Synthetic oligonucleotide
aaagtatgaattaggagccgctctgtttattggatgggcaggagcctcactgtgcataat

SEQ ID NO: 124 - DNA, 60 bp, Artificial Sequence
  Synthetic oligonucleotide
ggaagtcctggggtttttcctcttccttctttgtggtttctgttttgtaatttaagaaga

SEQ ID NO: 125 - DNA, 60 bp, Artificial Sequence
  Synthetic oligonucleotide
aacatcatccaagacttctacaatccgctggtggcctccgggcagaagcgggagatgggt

SEQ ID NO: 126 - DNA, 60 bp, Artificial Sequence
  Synthetic oligonucleotide
gatggctcaaccgtgactttgggctctgcttgcatcggtgttggccactgtccccattta

SEQ ID NO: 127 - DNA, 60 bp, Artificial Sequence
  Synthetic oligonucleotide
tccaggaagaaccctaaatttatggagaccgtggcggagaaagcgctgcaggaataccgc

SEQ ID NO: 128 - DNA, 60 bp, Artificial Sequence
  Synthetic oligonucleotide
cgctgagaggttgaccaggaaatacaacgagctgctaaagtcctaccagtggaagatgct

SEQ ID NO: 129 - DNA, 60 bp, Artificial Sequence
  Synthetic oligonucleotide
gtctacaggccaaaatgcgcacagttgattttcggtgtgttcctgtataacggcttgaaa

SEQ ID NO: 130 - DNA, 60 bp, Artificial Sequence
  Synthetic oligonucleotide
gtatttggccacccagcatttttgggccgaagaaaaaattcaaacctgttgtccagagac

SEQ ID NO: 131 - DNA, 60 bp, Artificial Sequence
  Synthetic oligonucleotide
aaaccatgaaggggccttttggctgaaatcaccacctgcctttggatgaaggactccgtt

SEQ ID NO: 132 - DNA, 60 bp, Artificial Sequence
  Synthetic oligonucleotide
tggatgaaagactccgttgggaataaatggccaaagcttataggactctgtgacaggttg

SEQ ID NO: 133 - DNA, 60 bp, Artificial Sequence
  Synthetic oligonucleotide
ttaccgctgcaatgacaccatcccagaggactatgagacccatcagctgcggcaagccta

SEQ ID NO: 134 - DNA, 60 bp, Artificial Sequence
  Synthetic oligonucleotide
gctgctgtggaaggacccgcgaaacgtgggttggaaggacaagaagtcctatcgttggtt

SEQ ID NO: 135 - DNA, 60 bp, Artificial Sequence
  Synthetic oligonucleotide
tatctctccatgttcagttccaaggagtcccagcggggcatgggctacatgcccaaacgt

SEQ ID NO: 136 - DNA, 60 bp, Artificial Sequence
  Synthetic oligonucleotide
tttctttgatcttctttttcttttctccccctcttttttgttctaaagaaaagtcatttt

SEQ ID NO: 137 - DNA, 60 bp, Artificial Sequence
  Synthetic oligonucleotide
ggatcgttttgttttgtttttaaagaaaggtgagattggcttggttcttcatgagcacat

SEQ ID NO: 138 - DNA, 60 bp, Artificial Sequence
  Synthetic oligonucleotide
agagctatatcaaatgtgctcatgaagaaccaagccaatctcacctttctttaaaaacaa

SEQ ID NO: 139 - DNA, 60 bp, Artificial Sequence
  Synthetic oligonucleotide
tcatgagcacatttgatatagctctttttctgtttttccttgctcatttcgttttgggga

SEQ ID NO: 140 - DNA, 60 bp, Artificial Sequence
  Synthetic oligonucleotide
agaccctggtgaaggaggtcgtacagaatttcgctaaggagtttgtgatcagtgatcgga

SEQ ID NO: 141 - DNA, 60 bp, Artificial Sequence
  Synthetic oligonucleotide
aacaatgtatgtgaaagtgtaaaatagaatgttactttagaatgactataaacattaaaa

SEQ ID NO: 142 - DNA, 60 bp, Artificial Sequence
  Synthetic oligonucleotide
tcacggccatagcttccaatacaagcacaggggagtttatagttctgatgtctttgacat

SEQ ID NO: 143 - DNA, 60 bp, Artificial Sequence
  Synthetic oligonucleotide
aagggcttcaatcatattggaggcttcaatcatcctttgatttgtactgagtactggttg

SEQ ID NO: 144 - DNA, 60 bp, Artificial Sequence
  Synthetic oligonucleotide
agaaaggcttaccttctgtcatcaagtgattgtatcatcctggatcgtcatttccaagga

SEQ ID NO: 145 - DNA, 60 bp, Artificial Sequence
  Synthetic oligonucleotide
tggatcgtcatttccaaggaactagcctttcttttcctaagcgtctgtatgtgttctaaa

SEQ ID NO: 146 - DNA, 60 bp, Artificial Sequence
  Synthetic oligonucleotide
cccaacttctctggcaactggaaaatcatccgatcggaaaacttcgaggaattgctcaaa

SEQ ID NO: 147 - DNA, 60 bp, Artificial Sequence
  Synthetic oligonucleotide
tgagggcctgagcaggaaagactggccctctggcttctacactttgtccctgtagcctat

SEQ ID NO: 148 - DNA, 60 bp, Artificial Sequence
  Synthetic oligonucleotide
ccctacaccaacaaagaggaatggctgcaagagcccagatcacccattccgggttcactc

SEQ ID NO: 149 - DNA, 60 bp, Artificial Sequence
  Synthetic oligonucleotide
cgttccaggagaaaaggctctacttcccagcctttccttgcccctgacatctggactctt

SEQ ID NO: 150 - DNA, 60 bp, Artificial Sequence
  Synthetic oligonucleotide
ttcgcccctcccttgttttatattttatgaagttagtgcgggctttgctgctccctggcc

SEQ ID NO: 151 - DNA, 60 bp, Artificial Sequence
  Synthetic oligonucleotide
ataacaacaactttggatttttatatataaactttgtgatttaaatttactgaatttaat

SEQ ID NO: 152 - DNA, 60 bp, Artificial Sequence
  Synthetic oligonucleotide
ctgtacttggataggctggctaacttgtaggaagagagcactgtatcgtatccttttgct

SEQ ID NO: 153 - DNA, 60 bp, Artificial Sequence
  Synthetic oligonucleotide
tgtacttggataggctggctaactcgtaggaagagagcactgtatggtatccttttgctt

SEQ ID NO: 154 - DNA, 60 bp, Artificial Sequence
  Synthetic oligonucleotide
cttatccaggcagctaaaaacctgatgaatgctgttgtcctcacggtgaaagcatcctat

SEQ ID NO: 155 - DNA, 60 bp, Artificial Sequence
  Synthetic oligonucleotide
ataggtgtagcttggagtgctggtatctaatataccattgtattcactaactaactcaaa

SEQ ID NO: 156 - DNA, 60 bp, Artificial Sequence
  Synthetic oligonucleotide
gtgaaaaatctggaagtgtaatggtagaacataaaacttgtattgcttctgtttcagtgc

SEQ ID NO: 157 - DNA, 60 bp, Artificial Sequence
  Synthetic oligonucleotide
gacttcatcatgctgcagaacaacgagcacagaattgcgcagtacctggccacttatggc

SEQ ID NO: 158 - DNA, 60 bp, Artificial Sequence
  Synthetic oligonucleotide
tgaccatcaacatgaagccccttcagctataccggaaaggtgtgatcaaggccacaccca

SEQ ID NO: 159 - DNA, 60 bp, Artificial Sequence
  Synthetic oligonucleotide
tagagaccctgtggcgcatcagtttctgggattttgtggacgtctccgtgcatgaactgc

SEQ ID NO: 160 - DNA, 60 bp, Artificial Sequence
  Synthetic oligonucleotide
ggcggggccggggggactctggtatctaattctttaatgattcctataaatctaatgaca

SEQ ID NO: 161 - DNA, 60 bp, Artificial Sequence
  Synthetic oligonucleotide
aaatttgctgtctaagattaatagcattcaaagatccccagacttcatagaatactcagg

SEQ ID NO: 162 - DNA, 60 bp, Artificial Sequence
  Synthetic oligonucleotide
caagaggcaaaggaatccatgtagtagatatcctctgcttaaaaactcactacggaggag

SEQ ID NO: 163 - DNA, 60 bp, Artificial Sequence
  Synthetic oligonucleotide
cgtacatgtcagaaaccattagcattgcatgcaggtttcatattctttctaagatggaaa

SEQ ID NO: 164 - DNA, 60 bp, Artificial Sequence
  Synthetic oligonucleotide
gtggtacaacgcctggaacgagaagcgcagggtctacgaagaatagggtgaaaaacctca

SEQ ID NO: 165 - DNA, 60 bp, Artificial Sequence
  Synthetic oligonucleotide
ccctcattttttggatagtcaccagaccgcaatggaaacgtcctaaggagccaaattcta

SEQ ID NO: 166 - DNA, 60 bp, Artificial Sequence
  Synthetic oligonucleotide
ttagataactccagcagaaaactgtaactgctatgtcttcaggaaaatgtagaagaaaga

SEQ ID NO: 167 - DNA, 60 bp, Artificial Sequence
  Synthetic oligonucleotide
cttcctgtgctgcttgtgtcaaatggaacctgccctctaaagcactttctttcctttact

SEQ ID NO: 168 - DNA, 60 bp, Artificial Sequence
  Synthetic oligonucleotide
cacgtgttctgaaaccactggtgtctgctcagatgtgttgggacaaaatgaaagtgactt

SEQ ID NO: 169 - DNA, 60 bp, Artificial Sequence
  Synthetic oligonucleotide
atgtggctccctcctgctgtcctacagtcacaacatggagtttgtctttttctctgacag

SEQ ID NO: 170 - DNA, 60 bp, Artificial Sequence
  Synthetic oligonucleotide
aggatttctactttggtcttcaagaaagctgtgccccagaacaccagagatttcaactta

SEQ ID NO: 171 - DNA, 60 bp, Artificial Sequence
  Synthetic oligonucleotide
ttcaagagatggaaccctaagtggagaatgagttattctaaggatttctactttggtctt

SEQ ID NO: 172 - DNA, 60 bp, Artificial Sequence
  Synthetic oligonucleotide
agtacaccttggagttttttcgaaatatgggttgggtttttgggctcttggttgatttaa

SEQ ID NO: 173 - DNA, 60 bp, Artificial Sequence
  Synthetic oligonucleotide
gtttatgctaaaaataacaccaaaatgtggtgaactcttaaggacttttcccttcaagtg

SEQ ID NO: 174 - DNA, 60 bp, Artificial Sequence
  Synthetic oligonucleotide
ttcactgaggatgataagccattattaagaagcgttgctaatgttatacagcaggctggg

SEQ ID NO: 175 - DNA, 60 bp, Artificial Sequence
  Synthetic oligonucleotide
actttttataagcattcttttaataaaggaaaattgtttttgaagtatacctcctttggg

SEQ ID NO: 176 - DNA, 60 bp, Artificial Sequence
  Synthetic oligonucleotide
gcacctgttacagagggaaggccaagtgctgcaagtgagctgagagtgaccagaagaaat

SEQ ID NO: 177 - DNA, 60 bp, Artificial Sequence
  Synthetic oligonucleotide
gggggtagttagggagagactacatgaaatggtgtgcccctattttctttctgatcctaa

SEQ ID NO: 178 - DNA, 60 bp, Artificial Sequence
  Synthetic oligonucleotide
agcaataaagtcatccactcctgcatctggttggtctttattgagcacctactatatgca

SEQ ID NO: 179 - DNA, 60 bp, Artificial Sequence
  Synthetic oligonucleotide
ctctgcacccccagcctatcccagaggccttgcaggtgaccagcagtgtcattgtattta

SEQ ID NO: 180 - DNA, 60 bp, Artificial Sequence
  Synthetic oligonucleotide
tgtgtagtgtatgttataatacaggctaagctgccacaataaaagagcttaaatactgtg

SEQ ID NO: 181 - DNA, 60 bp, Artificial Sequence
  Synthetic oligonucleotide
agatggggccaacaaaccgcctggacttctggagcccacgtccactctggtccgtgtgaa

SEQ ID NO: 182 - DNA, 60 bp, Artificial Sequence
  Synthetic oligonucleotide
aggcaggaaaacagccagaagccaccttgacacttttgaacatttccagttctgtagagt

SEQ ID NO: 183 - DNA, 60 bp, Artificial Sequence
  Synthetic oligonucleotide
aataacaccttccaaaaacctggagtgtaagagctttgtttctttatggaactcccctgt

SEQ ID NO: 184 - DNA, 60 bp, Artificial Sequence
  Synthetic oligonucleotide
atccatttactcagctggagaggagacatcaagaacatgccagacacatttcttttggtg

SEQ ID NO: 185 - DNA, 60 bp, Artificial Sequence
  Synthetic oligonucleotide
agtcatgagcagagctgaaaattttaaacaagttgagtacctccttattcatggaacagc

SEQ ID NO: 186 - DNA, 60 bp, Artificial Sequence
  Synthetic oligonucleotide
gtgccagttatggctataggtgctacaaaaacacagcaagggtgatgggaaagcattgta

SEQ ID NO: 187 - DNA, 60 bp, Artificial Sequence
  Synthetic oligonucleotide
aaatcatttccgcatcagctgctgaaacaacaaataggaattgtttttatggaagctttg

SEQ ID NO: 188 - DNA, 60 bp, Artificial Sequence
  Synthetic oligonucleotide
agtctaacaccacaactaatttcacccaaggttttaagcacgttctttcatcagaccctg

SEQ ID NO: 189 - DNA, 60 bp, Artificial Sequence
  Synthetic oligonucleotide
aaagcaaaggagtgaacttctaatgctgtaatttcagactcacatggttgcgcacatgga

SEQ ID NO: 190 - DNA, 60 bp, Artificial Sequence
  Synthetic oligonucleotide
gatgaacaggtcaactgcttcaacatcaattatctgaggaacgtggctctagtgtctgga

SEQ ID NO: 191 - DNA, 60 bp, Artificial Sequence
  Synthetic oligonucleotide
tggagggtcaaatggggggaaccccaccctaccccacccctttgaacactcattacagta

SEQ ID NO: 192 - DNA, 60 bp, Artificial Sequence
  Synthetic oligonucleotide
gaacagaaggaaggaatgccagctccatttggggaccagagccatccagaacctgagtcc

SEQ ID NO: 193 - DNA, 60 bp, Artificial Sequence
  Synthetic oligonucleotide
cattgcgcaaacactcagaaagtactgccaaaagcctaataaaaaatctaaagtttgctc

SEQ ID NO: 194 - DNA, 60 bp, Artificial Sequence; identical to SEQ ID NO: 193
  Synthetic oligonucleotide

SEQ ID NO: 195 - DNA, 60 bp, Artificial Sequence; identical to SEQ ID NO: 193
  Synthetic oligonucleotide

SEQ ID NO: 196 - DNA, 60 bp, Artificial Sequence
  Synthetic oligonucleotide
aagttattacaggtataaaagtgatgacctatcatgaggaaatgaaagtggctgatttgc

SEQ ID NO: 197 - DNA, 60 bp, Artificial Sequence
  Synthetic oligonucleotide
cagaaagaaagagcaataataattaattcacatgccatgtggattctatttataaatcac

SEQ ID NO: 198 - DNA, 60 bp, Artificial Sequence
  Synthetic oligonucleotide
gcttcactgaattcctgcattaacccaattgctctgtatttggtgagcaaaagattcaaa

SEQ ID NO: 199 - DNA, 60 bp, Artificial Sequence
  Synthetic oligonucleotide
gtaaaccaaaacccaacaatgtggccagaaagaaagagcaataataattaattcacacac

SEQ ID NO: 200 - DNA, 60 bp, Artificial Sequence
  Synthetic oligonucleotide
tgtgacaagtggacactatttatgttaaatatacaatcatcaaggaagtatgaagttatt

SEQ ID NO: 201 - DNA, 60 bp, Artificial Sequence
  Synthetic oligonucleotide
aggcagccatcataaccattgaatagcatgcaagggtaagaatgagtttttaactgcttt

SEQ ID NO: 202 - DNA, 60 bp, Artificial Sequence
  Synthetic oligonucleotide
ggaggcagccatcataaccattgaatagcatgcaagggtaagaatgagtttttaactgct

SEQ ID NO: 203 - DNA, 60 bp, Artificial Sequence
  Synthetic oligonucleotide
ggaaccactcccaccacaggcacaagctgtcacctagcagcctcaaaacgggtcagtatt

SEQ ID NO: 204 - DNA, 60 bp, Artificial Sequence
  Synthetic oligonucleotide
taaagggcagggcccacgtgtatagtatctgtatataagttgctgtgtgtctgtcctgat

SEQ ID NO: 205 - DNA, 60 bp, Artificial Sequence
  Synthetic oligonucleotide
atggagaaagaagtggagacagtcctttcccaccattcctgcctttaagccaaagaaaca

SEQ ID NO: 206 - DNA, 60 bp, Artificial Sequence
  Synthetic oligonucleotide
agttttccgggccaagaatttttatccatgaagactttcctacttttctcggtgttctta

SEQ ID NO: 207 - DNA, 60 bp, Artificial Sequence
  Synthetic oligonucleotide
tgtatggattagcatcagtgaagaacgttactcttttgtcaaggtatttggatttgctca

SEQ ID NO: 208 - DNA, 60 bp, Artificial Sequence
  Synthetic oligonucleotide
ttgtgaatctattgtttctcctctgaagcatttggtggcctaatttacaagcacgatgga

SEQ ID NO: 209 - DNA, 60 bp, Artificial Sequence
  Synthetic oligonucleotide
agattacacaaccttcacgacagttggtgattggctggatgccatcaagatggggcggta

SEQ ID NO: 210 - DNA, 60 bp, Artificial Sequence
  Synthetic oligonucleotide
aactggaggaggggactccaggaatggggaaatgtgacaccaccatcctgaagccagctt

SEQ ID NO: 211 - DNA, 60 bp, Artificial Sequence
  Synthetic oligonucleotide
ccaaacgagctgttcataacagcgattttgcctaacattatatcataagcgtgttccaat

SEQ ID NO: 212 - DNA, 60 bp, Artificial Sequence
  Synthetic oligonucleotide
ttgctccctgattaaaatgagatatggctatttggaagacactgcattttagccagtgta

SEQ ID NO: 213 - DNA, 60 bp, Artificial Sequence
  Synthetic oligonucleotide
taaatagggataagagaaactcttactatgcagattacgtttttgaatggtgaacaggct

SEQ ID NO: 214 - DNA, 60 bp, Artificial Sequence
  Synthetic oligonucleotide
aacacttggaggtgtgccttgtacgtcactcaacaaacactcagcagctgctaaaagaaa

SEQ ID NO: 215 - DNA, 60 bp, Artificial Sequence
  Synthetic oligonucleotide
gagttggtgttcataatttcagttctagttgattgcgagaattttcaaataaggaagagg

SEQ ID NO: 216 - DNA, 60 bp, Artificial Sequence
  Synthetic oligonucleotide
gtgaatagcaatatcccaactaaccttcgtgtgcttcgttcaatcctggaaaacctgaga

SEQ ID NO: 217 - DNA, 60 bp, Artificial Sequence
  Synthetic oligonucleotide
tgagcaaaggagcttaatgctaaggtcaaaaaggagagtgaaaggttgagaacaattgtc

SEQ ID NO: 218 - DNA, 60 bp, Artificial Sequence
  Synthetic oligonucleotide
tgtgatcatcagcaataaagatataataactctgttttcttagcctgtatagaggagagg

SEQ ID NO: 219 - DNA, 60 bp, Artificial Sequence
  Synthetic oligonucleotide
actgaagactttgaacacttgctttttgtgattgcttatgtcattagtgcctcatgactg

SEQ ID NO: 220 - DNA, 60 bp, Artificial Sequence
  Synthetic oligonucleotide
actcagctctcacaggggtaatcatctcaagtggtatttgtagccaagtgggagctattt

SEQ ID NO: 221 - DNA, 60 bp, Artificial Sequence
  Synthetic oligonucleotide
tccctcattcatcttgcaagcaaatcccatttcttgaaaagccttggagaactcggtttg

SEQ ID NO: 222 - DNA, 60 bp, Artificial Sequence
  Synthetic oligonucleotide
tgggactggaagtgacctgtacaagtgatgcagaaaggagggtttcaaagaaaaaggatt

SEQ ID NO: 223 - DNA, 60 bp, Artificial Sequence
  Synthetic oligonucleotide
tattggaagacttgggttgatctcttagaagccatgggacctcctccctcattcatcttg

SEQ ID NO: 224 - DNA, 60 bp, Artificial Sequence
  Synthetic oligonucleotide
tgctgtcactccgggcacctcgaacctcgtcttcacctatcctagcgtcctggagcagga

SEQ ID NO: 225 - DNA, 60 bp, Artificial Sequence
  Synthetic oligonucleotide
tgactatgagaccgttcgcaatgggggcctgatcttcgctggactggccttcatcgtggg

SEQ ID NO: 226 - DNA, 60 bp, Artificial Sequence
  Synthetic oligonucleotide
ctttagacctttgtccccgtcactgccagcgcttgggctgaaggaagctccagactcaat

SEQ ID NO: 227 - DNA, 60 bp, Artificial Sequence
  Synthetic oligonucleotide
ccaacactgtgtgaattatctaaatgcgtctaccattttgcactagggaggaaggataaa

SEQ ID NO: 228 - DNA, 60 bp, Artificial Sequence
  Synthetic oligonucleotide
gacgcgtccactactgtgtcaattatctaaatacgtctaccattttgcgctagggaggaa

SEQ ID NO: 229 - DNA, 60 bp, Artificial Sequence
  Synthetic oligonucleotide
gatctcttacctttggaaaataggggttaggcatgaaggtggttgtgattaagaagatgg

SEQ ID NO: 230 - DNA, 60 bp, Artificial Sequence
  Synthetic oligonucleotide
ggattggctttgatagaggaatggggatgatgtaagtttacagtattcctggggtttaat

SEQ ID NO: 231 - DNA, 60 bp, Artificial Sequence
  Synthetic oligonucleotide
tgtgtgtcagttctgtcagctgcaagttcttgtgtaatgaagtcaatgctgtcaggccaa

SEQ ID NO: 232 - DNA, 60 bp, Artificial Sequence
  Synthetic oligonucleotide
cctgccttaagtcaacttatttgtttttgccgggaaagtcgctacatggatcaatgggtt

SEQ ID NO: 233 - DNA, 60 bp, Artificial Sequence
  Synthetic oligonucleotide
ttgttccactcttacgagactgcaccaactcggatcatcagaaatggttcttcaaggagc

SEQ ID NO: 234 - DNA, 60 bp, Artificial Sequence
  Synthetic oligonucleotide
gatttgtgaatgatcccagaccaaccctgagattttgtcaacctgattaagtcaatatga

SEQ ID NO: 235 - DNA, 60 bp, Artificial Sequence
  Synthetic oligonucleotide
gagaatcagaagttcatcttgcaggaggatggatctttatttcacgaacagtccaagaaa

SEQ ID NO: 236 - DNA, 60 bp, Artificial Sequence
  Synthetic oligonucleotide
tccctataactcaaaataacttgtttgtaaaagaaaatttgtttacttacccattagtaa

SEQ ID NO: 237 - DNA, 60 bp, Artificial Sequence
  Synthetic oligonucleotide
acttttctaggatgaagacagcttatttttaagttgtatggtcttagttggtttagggtc

SEQ ID NO: 238 - DNA, 60 bp, Artificial Sequence
  Synthetic oligonucleotide
ggtatttgaaaaacgtttttcctaatttacatgttccagaggatagaccaggctggcatg

SEQ ID NO: 239 - DNA, 60 bp, Artificial Sequence
  Synthetic oligonucleotide
aaagctggggttcattttggtatatcacactgaaactgggtacccagagtgctgctgttt

SEQ ID NO: 240 - DNA, 60 bp, Artificial Sequence
  Synthetic oligonucleotide
cttggggacagatagaggggatggttggggatacttcccaaaactttttcaagtcaactt

SEQ ID NO: 241 - DNA, 60 bp, Artificial Sequence
  Synthetic oligonucleotide
ggattaagggtccaaaaatgctgatctaaggggttgccatggtgttgaacaatgcaactt

SEQ ID NO: 242 - DNA, 60 bp, Artificial Sequence
  Synthetic oligonucleotide
atggatggatggacttcggccgccgcagtgctgaggatgagaactaacaatcctagaacc

SEQ ID NO: 243 - DNA, 60 bp, Artificial Sequence
  Synthetic oligonucleotide
agcctatggatggatggacttcggccgccgcagtgctgaggatgagaactaacaatccta

SEQ ID NO: 244 - DNA, 60 bp, Artificial Sequence
  Synthetic oligonucleotide
gctgaagtgtggtgaatatgatgaaaatctgatgagaccaaacaagccatggggcacagt

SEQ ID NO: 245 - DNA, 60 bp, Artificial Sequence
  Synthetic oligonucleotide
atacaggccaaagaggtgctgaaaaaatatttggagtccaaggaggatgtggctgatgca

SEQ ID NO: 246 - DNA, 60 bp, Artificial Sequence
  Synthetic oligonucleotide
tattggggtgaccttcttggggactcgggggctggtctgatggaactgtgtatttattta

SEQ ID NO: 247 - DNA, 60 bp, Artificial Sequence
  Synthetic oligonucleotide
ttggggtgaccttcctgggaactggggggctggtctgatggaactgtgtatttatttaaa

SEQ ID NO: 248 - DNA, 60 bp, Artificial Sequence
  Synthetic oligonucleotide
aagggataaacctaaatatttacttgttatcattagagagggaacatcaaatgctgggac

SEQ ID NO: 249 - DNA, 60 bp, Artificial Sequence
  Synthetic oligonucleotide
cagctcaagtaccctaatttagttcttttggactaatacaattcaggaaagaaaaaaccc

SEQ ID NO: 250 - DNA, 60 bp, Artificial Sequence
  Synthetic oligonucleotide
gtgtgtaggacggggaggtcacgatggcgcgacgtctgcagaaatttcatgaggaggtat

SEQ ID NO: 251 - DNA, 60 bp, Artificial Sequence
  Synthetic oligonucleotide
tgtggaattcatccatccaattaattccagcttataatatttctggtccacatttgatgc

SEQ ID NO: 252 - DNA, 60 bp, Artificial Sequence
  Synthetic oligonucleotide
ccatagtaataatacacatttctgtgagtgctgacttgtctttgcaatatttcttctctg

SEQ ID NO: 253 - DNA, 60 bp, Artificial Sequence
  Synthetic oligonucleotide
accccttttagttaagtctttcactaaggttctcttgcatatatttcaagtgaatgttgg

SEQ ID NO: 254 - DNA, 60 bp, Artificial Sequence
  Synthetic oligonucleotide
agggaactgtcctacactgctattgttgctacatgtatcgagccttgattgctcctagtt

SEQ ID NO: 255 - DNA, 60 bp, Artificial Sequence
  Synthetic oligonucleotide
atgagaccatcctgaaagagcagaagggtcagagcatgttcgtggagaacaaggcctttt

SEQ ID NO: 256 - DNA, 60 bp, Artificial Sequence
  Synthetic oligonucleotide
cctgaaagagcagaagggtcagagcatgttcgtggagaacaaggccttttccatggatga

SEQ ID NO: 257 - DNA, 60 bp, Artificial Sequence
  Synthetic oligonucleotide
tccagccaaatagtgttctcggggtggtggctgggcagcgcctatgtttctctggagatt

SEQ ID NO: 258 - DNA, 60 bp, Artificial Sequence
  Synthetic oligonucleotide
gcaggaacaggtgtccatgaccctgattacctggatctcacaggttctgggaatgcaata

SEQ ID NO: 259 - DNA, 60 bp, Artificial Sequence
  Synthetic oligonucleotide
gggcctgctgtggctattccaacatctctggtcagggcctggaagtaccttaggacacat

SEQ ID NO: 260 - DNA, 60 bp, Artificial Sequence
  Synthetic oligonucleotide
aggcctggatcttgctcctctgtgaggaacaagggtgcctaataaaaacatttctgcttt

SEQ ID NO: 261 - DNA, 60 bp, Artificial Sequence
  Synthetic oligonucleotide
tgggtgtgggtgcatgtgggtgtttacacacatgcctacaggtatgcgtgattgtgtgtg

SEQ ID NO: 262 - DNA, 60 bp, Artificial Sequence
  Synthetic oligonucleotide
cactcaaggccccagcctggcacaaatggatgcatacagttctgtgtactgccaggcatg

SEQ ID NO: 263 - DNA, 60 bp, Artificial Sequence
  Synthetic oligonucleotide
tcgttgggaaattgatgaccggaaactagaaagcccctataatgtggaatatgcatatgt

SEQ ID NO: 264 - DNA, 60 bp, Artificial Sequence
  Synthetic oligonucleotide
ctgacgttactaagcattgcagcacaatgtagaaattggcttgggatggataggtatagg

SEQ ID NO: 265 - DNA, 60 bp, Artificial Sequence
  Synthetic oligonucleotide
gcaaacacgagcacactctcttcgaacccaattgtgggtgtagcaatgaaagcaatatga

SEQ ID NO: 266 - DNA, 60 bp, Artificial Sequence
  Synthetic oligonucleotide
aagtccactgttagtctctccgttagcaccagggacacacttgttctgagttttgttcat

SEQ ID NO: 267 - DNA, 60 bp, Artificial Sequence
  Synthetic oligonucleotide
ctccttctgggggttatcgtatgtacaaagtttaccttataatggctcaaattgtattta

SEQ ID NO: 268 - DNA, 60 bp, Artificial Sequence
  Synthetic oligonucleotide
gaatgaatatggtttgtgctggaagcctccgaggtggaaaaaactcgtgcaatggagatt

SEQ ID NO: 269 - DNA, 60 bp, Artificial Sequence
  Synthetic oligonucleotide
aaattaggcgccttgtttgagctgcatttcacacttctttagagctagctgacctttggc

SEQ ID NO: 270 - DNA, 60 bp, Artificial Sequence
  Synthetic oligonucleotide
aacccaaggaatgatggaatcaacacaacatagtatgtttgctttccttacccaattgta

SEQ ID NO: 271 - DNA, 60 bp, Artificial Sequence
  Synthetic oligonucleotide
ttctgcacaagaaatccaccaaaatcccggagtctgaggacctttaatgggctttgtcat

SEQ ID NO: 272 - DNA, 60 bp, Artificial Sequence
  Synthetic oligonucleotide
agatcaatccattgtatcattcagttcttctaaagcctacgttggttaggctgatggcag

SEQ ID NO: 273 - DNA, 60 bp, Artificial Sequence
  Synthetic oligonucleotide
ccaggtattttcagattgtaggagttttctttcttaacaatttcaacaggccactcactc

SEQ ID NO: 274 - DNA, 60 bp, Artificial Sequence
  Synthetic oligonucleotide
gcaacagataatcctatggtctttgccaataggggagagacaatttctggaggaaacttc

SEQ ID NO: 275 - DNA, 60 bp, Artificial Sequence
  Synthetic oligonucleotide
ttggggtcggtatctagtgctatccattcatctgtggtcgttccctctttgaagatgttt

SEQ ID NO: 276 - DNA, 60 bp, Artificial Sequence
  Synthetic oligonucleotide
tctgaggactccttgcatttggaatcatccggtttatttatgtgcaatttccttcccctc

SEQ ID NO: 277 - DNA, 60 bp, Artificial Sequence
  Synthetic oligonucleotide
ataggtttgtactgtgcctaatttactttgtaaaccagaatgattccgtttttgcctcac

SEQ ID NO: 278 - DNA, 60 bp, Artificial Sequence
  Synthetic oligonucleotide
aaacgacctccactgaactgggtttgacctctgttgtactgatgtgtcgtgactaaataa

SEQ ID NO: 279 - DNA, 60 bp, Artificial Sequence
  Synthetic oligonucleotide
gggctataaaatatcatttttcaggtttattcttttagcaggtgtagttaaacgacctcc

SEQ ID NO: 280 - DNA, 60 bp, Artificial Sequence
  Synthetic oligonucleotide
taaaagataagagacacaacatgtattatgcacttcatttctctactgtgtggagaaagc

SEQ ID NO: 281 - DNA, 60 bp, Artificial Sequence
  Synthetic oligonucleotide
aaaggagagggattatagagaaaaggcgtcgggatcggataaataacagtttatctgagt

SEQ ID NO: 282 - DNA, 60 bp, Artificial Sequence
  Synthetic oligonucleotide
aagctctttgtcaagttagtgattgcatttgatcccaaaacaagatgaatgtatgcaatg

SEQ ID NO: 283 - DNA, 60 bp, Artificial Sequence
  Synthetic oligonucleotide
tactcggggcaaagtactagctctgtgattagattgaattctccaacaacaacatctcag

SEQ ID NO: 284 - DNA, 60 bp, Artificial Sequence
  Synthetic oligonucleotide
ttccagtcccaaatcatttacttttctgtggtccagccctactcctataagtcatgatct

SEQ ID NO: 285 - DNA, 60 bp, Artificial Sequence
  Synthetic oligonucleotide
ggtagggaggcaggaagagggaaacattgtgtcttgtttaggatccttattgtgtgtatc

SEQ ID NO: 286 - DNA, 60 bp, Artificial Sequence
  Synthetic oligonucleotide
ccaaatcatttacttttctgtggtccagccctactcctataagtcatgatctccaaagct

SEQ ID NO: 287 - DNA, 60 bp, Artificial Sequence; identical to SEQ ID NO: 286
  Synthetic oligonucleotide

SEQ ID NO: 288 - DNA, 60 bp, Artificial Sequence
  Synthetic oligonucleotide
tgtcttccaactgcagtctccacagtcttcagaagacaaatgctcaggtagtcactgttt

SEQ ID NO: 289 - DNA, 60 bp, Artificial Sequence
  Synthetic oligonucleotide
ggtctctcggaagccaccgtgtggttctttcacaggcacgtttattttgctgaaataaaa

SEQ ID NO: 290 - DNA, 60 bp, Artificial Sequence
  Synthetic oligonucleotide
agacaaagtagtgaacatcaatgaacatctgatagagataaactgtaatcaggcataagc

SEQ ID NO: 291 - DNA, 60 bp, Artificial Sequence
  Synthetic oligonucleotide
catcattatcaatatgtcatctttagcaggactcatgcccgttgcacagcagccggttta

SEQ ID NO: 292 - DNA, 60 bp, Artificial Sequence
  Synthetic oligonucleotide
cctctgctcactttaagaactttaactgactccaaaaatctcaggaattaaactgttaac

SEQ ID NO: 293 - DNA, 60 bp, Artificial Sequence
  Synthetic oligonucleotide
agtggatgaaaacttcgatcctttacctgattattggctatctcttctgttcaagaaatt

SEQ ID NO: 294 - DNA, 60 bp, Artificial Sequence
  Synthetic oligonucleotide
catcaagggggtcttgttttgctagagagtttggggtttggtttgtggatttcattgtga

SEQ ID NO: 295 - DNA, 60 bp, Artificial Sequence
  Synthetic oligonucleotide
tgcagatagtgcctctgcaaactaaggagtgactaggtgggttggggaccccctcaggat

SEQ ID NO: 296 - DNA, 60 bp, Artificial Sequence
  Synthetic oligonucleotide
aaagccaccggaggaaaggaaaaaacatcggccaacctagaaacgttttcattcgtcatt

SEQ ID NO: 297 - DNA, 60 bp, Artificial Sequence
  Synthetic oligonucleotide
gttctggtgtcatagatgtcccattttgtgaggtagagctgtgcattaaacttgcacatg

SEQ ID NO: 298 - DNA, 60 bp, Artificial Sequence
  Synthetic oligonucleotide
gcacagggaagttctggtgtcatagatatcccgttttgtgaggtagagctgtgcattaaa

SEQ ID NO: 299 - DNA, 60 bp, Artificial Sequence; identical to SEQ ID NO: 298
  Synthetic oligonucleotide

SEQ ID NO: 300 - DNA, 60 bp, Artificial Sequence
  Synthetic oligonucleotide
cgactgtcgagattgcccagtatgttctgtgaacacaaataaacttgatttactgtctgc

SEQ ID NO: 301 - DNA, 60 bp, Artificial Sequence
  Synthetic oligonucleotide
tgtgagatgttccccctgctgtaaatgcaggtctcttggtatttattgagctttgtggga

SEQ ID NO: 302 - DNA, 60 bp, Artificial Sequence; identical to SEQ ID NO: 300
  Synthetic oligonucleotide

SEQ ID NO: 303 - DNA, 60 bp, Artificial Sequence
  Synthetic oligonucleotide
gtacgtaatatttattttaacttatgcaagggtgtgagatgttccccctgctgtaaatgc

SEQ ID NO: 304 - DNA, 60 bp, Artificial Sequence
  Synthetic oligonucleotide
gccacaacaaccctcttaaaactaattggctttttagaaacaccccacaaaagctcagaa

SEQ ID NO: 305 - DNA, 60 bp, Artificial Sequence
  Synthetic oligonucleotide
tgcttccggacaacttccccagataccccgtgggcaagttcttccaatatgacacctgga

SEQ ID NO: 306 - DNA, 60 bp, Artificial Sequence
  Synthetic oligonucleotide
ctcaactcagctcctttaacgctaatatttccggcaaaatcccatgcttgggttttgtct

SEQ ID NO: 307 - DNA, 60 bp, Artificial Sequence
  Synthetic oligonucleotide
catgaaacacttctcatcatattgtatgtaagtaattgcatttctgctcttccaaagctc

SEQ ID NO: 308 - DNA, 60 bp, Artificial Sequence
  Synthetic oligonucleotide
tttctgctcttccaaagctcctgcgtctgtttttaaagagcatggaaaaatactgcctag

SEQ ID NO: 309 - DNA, 60 bp, Artificial Sequence
  Synthetic oligonucleotide
aatcaaagctacctgtggtgatgttgccaccggttaaaatgtacactggatatgttgtta

SEQ ID NO: 310 - DNA, 60 bp, Artificial Sequence
  Synthetic oligonucleotide
ctgtgtggaaccactgactactggctctcattgacttccttactaagcatagcaaacaga

SEQ ID NO: 311 - DNA, 60 bp, Artificial Sequence
  Synthetic oligonucleotide
ttgaggttgtctgagtcttgggtctatgccttgaaaaaagctgaattattggacagtctc

SEQ ID NO: 312 - DNA, 60 bp, Artificial Sequence
  Synthetic oligonucleotide
ccacaaagtggacgcctgctgtatcttcccaacagtggcttcacagacccacaagagaag

SEQ ID NO: 313 - DNA, 60 bp, Artificial Sequence
  Synthetic oligonucleotide
ctcccggatgccttggagatagaggcctgccaggtgtactttacttacgacccctactca

SEQ ID NO: 314 - DNA, 60 bp, Artificial Sequence
  Synthetic oligonucleotide
tccccccagaatataaatctcaggtaataaggctttagaactgctgataaagcggatcgt

SEQ ID NO: 315 - DNA, 60 bp, Artificial Sequence
  Synthetic oligonucleotide
tagctgtttctctctttaatctcacgtagcctttttcaggttagtacgtgttcttctgtc

SEQ ID NO: 316 - DNA, 60 bp, Artificial Sequence
  Synthetic oligonucleotide
atctgcaaatcgtgactaagtacatcctgattcctgcaagccagcagccaaaggagaata

SEQ ID NO: 317 - DNA, 60 bp, Artificial Sequence
  Synthetic oligonucleotide
acccgcgagttcgggctcctgctgctcttcctctgcgtggccatcgccctcttcgcgccc

SEQ ID NO: 318 - DNA, 60 bp, Artificial Sequence
  Synthetic oligonucleotide
ccctatgtgtgtttccctcaataaggagatgccttgttcttttcaccatgcaaataacat

SEQ ID NO: 319 - DNA, 60 bp, Artificial Sequence
  Synthetic oligonucleotide
cctgtcggtgctcttcgtgaccgtcaccgccgtcaacctctccgtcagcaccttgcccag

SEQ ID NO: 320 - DNA, 60 bp, Artificial Sequence
  Synthetic oligonucleotide
ccagcccctatgtgtgtttccctcaataaggagatgccttgttcttttcaccatgcaaat

SEQ ID NO: 321 - DNA, 60 bp, Artificial Sequence
  Synthetic oligonucleotide
ccgccgatgcacccgcgagttcgggctcctgctgctcttcctctgcgtggccatcgccct

SEQ ID NO: 322 - DNA, 60 bp, Artificial Sequence
  Synthetic oligonucleotide
aagttgaacaatcctagccattgacaatcgtgatagttattattttcccatttgctgtct

SEQ ID NO: 323 - DNA, 60 bp, Artificial Sequence
  Synthetic oligonucleotide
tcgttttgtgtttttcccaaaacttgaacttgcaggcaagccttggttgggtatttgatt

SEQ ID NO: 324 - DNA, 60 bp, Artificial Sequence
  Synthetic oligonucleotide
cctgcgccattgcctgctgcctgtgctcagccgcccacgtgcctgggtagcggtccactg

SEQ ID NO: 325 - DNA, 60 bp, Artificial Sequence
  Synthetic oligonucleotide
gcttgctgaagcgtcaggtgaccgagttcagctcccataaggtggcggcacctaaggagg

SEQ ID NO: 326 - DNA, 60 bp, Artificial Sequence
  Synthetic oligonucleotide
cttttgtaactcaagtcttgaaatgttctgtagtgttaagcaaagtctcctcttgcttga

SEQ ID NO: 327 - DNA, 60 bp, Artificial Sequence
  Synthetic oligonucleotide
atgatgaacagagttatgattcctatgataacagctatagcaccccagcccaaagtggtg

SEQ ID NO: 328 - DNA, 60 bp, Artificial Sequence
  Synthetic oligonucleotide
aaacacacagctgctgaatgttcaacctgtgaaactgagatgtttctagaatgaaacagt

SEQ ID NO: 329 - DNA, 60 bp, Artificial Sequence
  Synthetic oligonucleotide
aggataccaaatggaaacacatgatgatgcctctgggtctgtatgagaccgtgatgaagt

SEQ ID NO: 330 - DNA, 60 bp, Artificial Sequence
  Synthetic oligonucleotide
ctcaaagttttcttaagggaaaacactacaaaaagtcacaaggataccaaatggaaacac

SEQ ID NO: 331 - DNA, 60 bp, Artificial Sequence
  Synthetic oligonucleotide
cacatagtcctttgtgaacttgtttgtgaaggaagttcactttttgtgtacatacgtgta

SEQ ID NO: 332 - DNA, 60 bp, Artificial Sequence
  Synthetic oligonucleotide
ttgcaaggcattagaaaaaatttcacaattacaggggactgaaaatgtgatttcaaccag

SEQ ID NO: 333 - DNA, 60 bp, Artificial Sequence
  Synthetic oligonucleotide
acactccaagacctgtgccttttagagaagctcacaatgatttaaggactgtttgaaact

SEQ ID NO: 334 - DNA, 60 bp, Artificial Sequence
  Synthetic oligonucleotide
aacacactggagaggtctaaagtggaagaaactacagagcacttggttacaaagagcaga

SEQ ID NO: 335 - DNA, 60 bp, Artificial Sequence
  Synthetic oligonucleotide
tcaagccttgaccacttgtgatgacatcttaatcaaacaggaccagactctggctgaact

SEQ ID NO: 336 - DNA, 60 bp, Artificial Sequence
  Synthetic oligonucleotide
gcttgggagatgctttcaggttgcagccagaaggggttttttaaatgacttctctggatt

SEQ ID NO: 337 - DNA, 60 bp, Artificial Sequence
  Synthetic oligonucleotide
ggttgaaataggagatgacctctaactgatagaacgttactttgtgtcgtgatgaaaact

SEQ ID NO: 338 - DNA, 60 bp, Artificial Sequence
  Synthetic oligonucleotide
attgttgggatcctgggaattatctgtcttatcttaatggcctctgtggtaacgatagtt

SEQ ID NO: 339 - DNA, 60 bp, Artificial Sequence
  Synthetic oligonucleotide
agtggaatattaagtaaaagttgggcactaatctggattaacattcgaggaaatcagttg

SEQ ID NO: 340 - DNA, 60 bp, Artificial Sequence
  Synthetic oligonucleotide
ataggacagatcccatctcctccacccaatacattattagactgaactgtgacctgaaat

SEQ ID NO: 341 - DNA, 60 bp, Artificial Sequence
  Synthetic oligonucleotide
tcatgcgattatgccatcgacctttatccctctctatacaatggtcactttttccagaat

SEQ ID NO: 342 - DNA, 60 bp, Artificial Sequence
  Synthetic oligonucleotide
aaagtgggaaataacttgcaacgtctgttagagatggttgctacacatgttgggtctgta

SEQ ID NO: 343 - DNA, 60 bp, Artificial Sequence
  Synthetic oligonucleotide
agaatgaagataaaatgttgatcatgtatatatatccatagtgaataaaattgtctcagt

SEQ ID NO: 344 - DNA, 60 bp, Artificial Sequence
  Synthetic oligonucleotide
agtgtattcgacaactctgtgagtttcttacagaaaatggttatgcacataatgtgtcca

SEQ ID NO: 345 - DNA, 60 bp, Artificial Sequence
  Synthetic oligonucleotide
gaagatgaaattaaccgccgcacagctgctgagaatgagtttgtggtgctgaagaaggat

SEQ ID NO: 346 - DNA, 60 bp, Artificial Sequence
  Synthetic oligonucleotide
gaagatgaaattaaccaccgcacagctgctgagaatgagtttgtggtgctgaagaaggat

SEQ ID NO: 347 - DNA, 60 bp, Artificial Sequence
  Synthetic oligonucleotide
gcctttagttctccactggggaggaatcctggaccaagcacaaaaacttaacaaaagtga

SEQ ID NO: 348 - DNA, 60 bp, Artificial Sequence
  Synthetic oligonucleotide
atggccaggttgcctacgagtggggtccactgatgaaaagaggttttttgtacttacata

SEQ ID NO: 349 - DNA, 60 bp, Artificial Sequence
  Synthetic oligonucleotide
ccaggttgcctaggagtggggtccactgatgaaaagaggtgttttgtacttacataagaa

SEQ ID NO: 350 - DNA, 60 bp, Artificial Sequence
  Synthetic oligonucleotide
catttccctgcagatggtacagatgttcctgccttagagtcatctctagttccccacctc

SEQ ID NO: 351 - DNA, 60 bp, Artificial Sequence
  Synthetic oligonucleotide
gtcttccagaagaagaagctgggctgtcaactcctggggaagtataagcagatcatcgcc

SEQ ID NO: 352 - DNA, 60 bp, Artificial Sequence
  Synthetic oligonucleotide
tctggcacccggggaggtagcatttccctgcagatggtacagatgttcctgccttagagt

SEQ ID NO: 353 - DNA, 60 bp, Artificial Sequence
  Synthetic oligonucleotide
aggagcaggctgggatcccaactatcgcttgttgcctcttttcaagtggaatttgaattt

SEQ ID NO: 354 - DNA, 60 bp, Artificial Sequence
  Synthetic oligonucleotide
aggagcaggctgggatcccaactatcgcttgttgcctctttttcaagtggaatttgaatt

SEQ ID NO: 355 - DNA, 60 bp, Artificial Sequence
  Synthetic oligonucleotide
acacactggctcctagacctaaagggtatgagctggagctaaggccagctagagcttcca

SEQ ID NO: 356 - DNA, 60 bp, Artificial Sequence
  Synthetic oligonucleotide
acttgaaggtttgatgccaaagcagacattttcctcacacccacctgctgctgtatgaat

SEQ ID NO: 357 - DNA, 60 bp, Artificial Sequence
  Synthetic oligonucleotide
ggtgtgatggacgggcagcttcctgtgtgctccaagggatgagcctcgtggggcagaggg

SEQ ID NO: 358 - DNA, 60 bp, Artificial Sequence
  Synthetic oligonucleotide
ctttcctcggttctggcctccagaccagagtaaggggcaggtccctccaacaggtgctca

SEQ ID NO: 359 - DNA, 60 bp, Artificial Sequence
  Synthetic oligonucleotide
agcactgatttactctgtaaaaagcaaaatctctctgtcctaaactaatggaagcgattc

SEQ ID NO: 360 - DNA, 60 bp, Artificial Sequence
  Synthetic oligonucleotide
tctggctccctggatgacacagagacggagcagctgttacaggaagagcagtctgagtgt

SEQ ID NO: 361 - DNA, 60 bp, Artificial Sequence
  Synthetic oligonucleotide
ttttttctgaataagtctctcataatgagtgcagtgtcagactgtgcctactctgatggt

SEQ ID NO: 362 - DNA, 60 bp, Artificial Sequence
  Synthetic oligonucleotide
ttaagggatattgggaaaagttttggtgtgtttctgttgacttcttttttgtatgctgtg

SEQ ID NO: 363 - DNA, 60 bp, Artificial Sequence
  Synthetic oligonucleotide
aggaagcttatagtgggtcaaacaaggaggtgtttagtgtgttgtttaaaaagaaggctc

SEQ ID NO: 364 - DNA, 60 bp, Artificial Sequence
  Synthetic oligonucleotide
gcaccactacaatgtccattttaatggcctggaatgcaaaacgcctgaagaatacaaagg

SEQ ID NO: 365 - DNA, 60 bp, Artificial Sequence
  Synthetic oligonucleotide
ctgctgaagatcattggattttccacatcagtaactgccctggggtttgtgctgtacaaa

SEQ ID NO: 366 - DNA, 60 bp, Artificial Sequence
  Synthetic oligonucleotide
ccccagtgctacacgttggagtatacctatgtgtgtgctttgccactgaagtaagatttt

SEQ ID NO: 367 - DNA, 60 bp, Artificial Sequence
  Synthetic oligonucleotide
ttttctttcttggcctcaagttcaatatggagagggattgcttccctgaatcctctcttc

SEQ ID NO: 368 - DNA, 60 bp, Artificial Sequence
  Synthetic oligonucleotide
acagcatcaatagaaagtcatctttgagataatttaaccctgcctctcagagggttttct

SEQ ID NO: 369 - DNA, 60 bp, Artificial Sequence
  Synthetic oligonucleotide
gtctgcttggatttcctacagcccccgtgggcatggaccacctttattttatacaaaatt

SEQ ID NO: 370 - DNA, 60 bp, Artificial Sequence
  Synthetic oligonucleotide
ccgcctctactccatacacaggccttgcaaacagtgtctcaacgaggtctgcttctacag

SEQ ID NO: 371 - DNA, 60 bp, Artificial Sequence
  Synthetic oligonucleotide
tgagaggaaggaggatctccttcttctccaaccattgacagctaacccttagacagtatt

SEQ ID NO: 372 - DNA, 60 bp, Artificial Sequence
  Synthetic oligonucleotide
ttgaccctcctactccacattgcaacatttgcatcagacagcatttcaattccagtatta

SEQ ID NO: 373 - DNA, 60 bp, Artificial Sequence
  Synthetic oligonucleotide
agatgactgtttctcatgcctttatcttccttcatgtaagtaaagtggacctttgtgctc

SEQ ID NO: 374 - DNA, 60 bp, Artificial Sequence
  Synthetic oligonucleotide
tattcaacctgtcctttcagggagtttattggaggatcaaagaactgaaagcactagagc

SEQ ID NO: 375 - DNA, 60 bp, Artificial Sequence
  Synthetic oligonucleotide
ttctcaatctaaatgcctttcaggtgggccgcttccttggctacctggttccagggggct

SEQ ID NO: 376 - DNA, 60 bp, Artificial Sequence
  Synthetic oligonucleotide
aagtggctggaagagtcccttagtactcttctagcatttagatctacactctcgagttaa

SEQ ID NO: 377 - DNA, 60 bp, Artificial Sequence
  Synthetic oligonucleotide
aacatttgctctgggggggcagggaatacacagatgcgttgcaaaggtaggttgaaggga

SEQ ID NO: 378 - DNA, 60 bp, Artificial Sequence
  Synthetic oligonucleotide
accgccgcgccaaggcaaagagactacaagaggcagagctggagaagctgaagatggccg

SEQ ID NO: 379 - DNA, 60 bp, Artificial Sequence
  Synthetic oligonucleotide
agctgctgtgcctgatgtcgggacagccctgctcccaagtacaaatagagtgacccgtaa

SEQ ID NO: 380 - DNA, 60 bp, Artificial Sequence
  Synthetic oligonucleotide
ttgggaactctagtctcgcctcgggttgcaatggaccccaactgctcctgtgccgctggt

SEQ ID NO: 381 - DNA, 60 bp, Artificial Sequence
  Synthetic oligonucleotide
gcatcggagaagtgcagctgctgcgcctgatgtcgggacagccctgctcccaagtacaaa

SEQ ID NO: 382 - DNA, 60 bp, Artificial Sequence
  Synthetic oligonucleotide
tgtgccaagtgtgcccacggctgcatctgcaaagggacgtcggagaagtgcagctgctgt

SEQ ID NO: 383 - DNA, 60 bp, Artificial Sequence
  Synthetic oligonucleotide
caaccctgaccgtgaccgtttgctatattcctttttctatgaaataatgtgaatgataat

SEQ ID NO: 384 - DNA, 60 bp, Artificial Sequence
  Synthetic oligonucleotide
cgctcccagatgtaaagaacgcgacttccacaaacctggattttttatgtacaaccctga

SEQ ID NO: 385 - DNA, 60 bp, Artificial Sequence
  Synthetic oligonucleotide
cacaatgattccaacttcaactctttcccttggtttactagagactacaggcttactggc

SEQ ID NO: 386 - DNA, 60 bp, Artificial Sequence
  Synthetic oligonucleotide
gagcacacgctagttcagaaagtccaagcaccatcaaacttaccatggcttcagtagtaa

SEQ ID NO: 387 - DNA, 60 bp, Artificial Sequence
  Synthetic oligonucleotide
tggtgtctttatctgcagtaacctcaccttcgccactttattccacaccatctgagagta

SEQ ID NO: 388 - DNA, 60 bp, Artificial Sequence
  Synthetic oligonucleotide
ccaagacctccttggttgaaacaactgatggaacgctagtgaccaccataaagatgtcaa

SEQ ID NO: 389 - DNA, 60 bp, Artificial Sequence
  Synthetic oligonucleotide
cagaatcagcagaaatgatgatcaagacacaaacagatcctcctgggtctacaccagaga

SEQ ID NO: 390 - DNA, 60 bp, Artificial Sequence
  Synthetic oligonucleotide
cagggatccactcagctatgactcatggattttcacaattggatgtgaccactcttatga

SEQ ID NO: 391 - DNA, 60 bp, Artificial Sequence
  Synthetic oligonucleotide
tatccctttttccatgacactaagcaatgcagaaacaagtgccgaaagggtcagaagcac

SEQ ID NO: 392 - DNA, 60 bp, Artificial Sequence
  Synthetic oligonucleotide
agcatcaagagttatttttctgactgtcaagtttcaacattcaggtctgtccccaacagg

SEQ ID NO: 393 - DNA, 60 bp, Artificial Sequence
  Synthetic oligonucleotide
tgagtcctgggatcagacaccccttcacgtgtatccccacacaaatgcaagctcaccaag

SEQ ID NO: 394 - DNA, 60 bp, Artificial Sequence
  Synthetic oligonucleotide
tgaggaagtggacgagatgtaccgggaggcacccattgataagaaaggcaacttcaacta

SEQ ID NO: 395 - DNA, 60 bp, Artificial Sequence
  Synthetic oligonucleotide
tgtgctcaggagttgcgggcagcatggacatctgtcccagaggaggcagaatctccaata

SEQ ID NO: 396 - DNA, 60 bp, Artificial Sequence
  Synthetic oligonucleotide
ttttcagccaggaagagctctctggaaattctgagttgatacaaaagtacagaaatatca

SEQ ID NO: 397 - DNA, 60 bp, Artificial Sequence
  Synthetic oligonucleotide
cgtttggctgcactaactttggtagctcagtgtgcatctagagtgggactggggagggag

SEQ ID NO: 398 - DNA, 60 bp, Artificial Sequence
  Synthetic oligonucleotide
gttttgagcgttgtattccaaaggcctcatctggagcctcgggaaagtctggtcccacat

SEQ ID NO: 399 - DNA, 60 bp, Artificial Sequence
  Synthetic oligonucleotide
aggaagctgagcagatccctgtgatgcctgtgacctcaattaaagcaattcctttgacct

SEQ ID NO: 400 - DNA, 60 bp, Artificial Sequence
  Synthetic oligonucleotide
ctgctgtgaaagaggctggctacacaatcgaatggtttgaggtgatctcgcaaagttatt

SEQ ID NO: 401 - DNA, 60 bp, Artificial Sequence
  Synthetic oligonucleotide
ctactagctctttgagataatacattccgaggggctcagttctgccttatctaaatcacc

SEQ ID NO: 402 - DNA, 60 bp, Artificial Sequence
  Synthetic oligonucleotide
agcagcttttgactgtttccagagtgcttataatatacataactccctggaaattactga

SEQ ID NO: 403 - DNA, 60 bp, Artificial Sequence
  Synthetic oligonucleotide
ctaaatgttgcgtgggtggcatgagttgaagaaggcaaaggcttgtaaatttacccaatg

SEQ ID NO: 404 - DNA, 60 bp, Artificial Sequence
  Synthetic oligonucleotide
aagttttctgctgtaaagaaagctgtaatatatagtaaaactaaatgttgcgtgggtggc

SEQ ID NO: 405 - DNA, 60 bp, Artificial Sequence
  Synthetic oligonucleotide
tataacaaaatgttttattttcattttagcaaaaattgtcttataatactagctaacggc

SEQ ID NO: 406 - DNA, 60 bp, Artificial Sequence
  Synthetic oligonucleotide
gctaacggcaaagacgtttttatagggaaactatttatatgtaacatcctgatttacagc

SEQ ID NO: 407 - DNA, 60 bp, Artificial Sequence
  Synthetic oligonucleotide
catctgcgtggctctgctggtcgtgggcatcgtctgtgtggtggcctactgcaagaccaa

SEQ ID NO: 408 - DNA, 60 bp, Artificial Sequence
  Synthetic oligonucleotide
ggacagagatgtttggagaaactgcctttgcgattgtacatgccagatcctaagcaaagt

SEQ ID NO: 409 - DNA, 60 bp, Artificial Sequence
  Synthetic oligonucleotide
ctagctaacggcaaaggcgtttttatagggaaactatttatatgtaacatcctgatttac

SEQ ID NO: 410 - DNA, 60 bp, Artificial Sequence
  Synthetic oligonucleotide
ctgcaatctagtgacaaagtcgaaagtttaacaggcttttctcatgaagaactagacgac

SEQ ID NO: 411 - DNA, 60 bp, Artificial Sequence
  Synthetic oligonucleotide
tagttcccctgattctgaccttgagtttgtagccaatactaaggcaagggtcaaagagct

SEQ ID NO: 412 - DNA, 60 bp, Artificial Sequence
  Synthetic oligonucleotide
ttttctcacgaagaaccagatgactcttggtaaccatgtttgctgcccagcttctaactt

SEQ ID NO: 413 - DNA, 60 bp, Artificial Sequence
  Synthetic oligonucleotide
aagggccctccaagccttaatggcacccttaagcctccatgcccaggccaaaagatgctt

SEQ ID NO: 414 - DNA, 60 bp, Artificial Sequence
  Synthetic oligonucleotide
gggctaacttaaaagagttttttcaatgctgcagtgactgaagaagcagtccactcccat

SEQ ID NO: 415 - DNA, 60 bp, Artificial Sequence
  Synthetic oligonucleotide
agggctatgcctgtgtcttattgagacaccttggcaaagagatggctgattctgggtggt

SEQ ID NO: 416 - DNA, 60 bp, Artificial Sequence
  Synthetic oligonucleotide
tcaaaggaattactctcttcttgttaaattagctaaatcatgtaaccgcagataggaagg

SEQ ID NO: 417 - DNA, 60 bp, Artificial Sequence
  Synthetic oligonucleotide
tttttcctttgatgttcaagtcctagtctataggattggcagtttaaatgctttactccc

SEQ ID NO: 418 - DNA, 60 bp, Artificial Sequence
  Synthetic oligonucleotide
ttagatatctgcaggggtgtctaaaagtgtgttcattttgcagcaatgtttaggtgcata

SEQ ID NO: 419 - DNA, 60 bp, Artificial Sequence
  Synthetic oligonucleotide
ttgaaggcaaagatcatcaatatctgcatctggctgctgtcgtcatctgttggcatctct

SEQ ID NO: 420 - DNA, 60 bp, Artificial Sequence
  Synthetic oligonucleotide
cagttcttaaacgtgtataactattgtcagacaatttataggtgtttcatctagtcctgg

SEQ ID NO: 421 - DNA, 60 bp, Artificial Sequence
  Synthetic oligonucleotide
gaaaatcacatccaaaaacggtataacccagcttccttaaggcaattttcttctctgaaa

SEQ ID NO: 422 - DNA, 60 bp, Artificial Sequence
  Synthetic oligonucleotide
tagtcgtggagatgtcttcgtacagttcttcaggaagagaggagttcaatgatctgggtt

SEQ ID NO: 423 - DNA, 60 bp, Artificial Sequence
  Synthetic oligonucleotide
tacggtggcgaacgaggccacgctgctcgatactgactacgacaatttcctgtttctctg

SEQ ID NO: 424 - DNA, 60 bp, Artificial Sequence
  Synthetic oligonucleotide
caaggccccctctgtccttttcagaacacatggacttggaggcagatttgaaataaactt

SEQ ID NO: 425 - DNA, 60 bp, Artificial Sequence
  Synthetic oligonucleotide
gccatgttgccacatgagcaagcttgggtgctcccaaggttcaaatactttttattagac

SEQ ID NO: 426 - DNA, 60 bp, Artificial Sequence
  Synthetic oligonucleotide
tttccctccaagctcctattttactgtgtcagctggaaggaaacctttccctcttgggac

SEQ ID NO: 427 - DNA, 60 bp, Artificial Sequence
  Synthetic oligonucleotide
ttactgtgtcagctggaaggaaacctttccctcttgggacctctttaccctctgtgacct

SEQ ID NO: 428 - DNA, 60 bp, Artificial Sequence
  Synthetic oligonucleotide
agctctggaaacagatgtctagtgatcatctcagctgaagtgtggcttgcgtaaataact

SEQ ID NO: 429 - DNA, 60 bp, Artificial Sequence
  Synthetic oligonucleotide
attggctataagcacttggaattgtactgggttttctgtaaagttttagaaactagctac

SEQ ID NO: 430 - DNA, 60 bp, Artificial Sequence
  Synthetic oligonucleotide
cttcagggaagatggctatcagatgaatgcacaaatgctgtggtgaacttcttatccaga

SEQ ID NO: 431 - DNA, 60 bp, Artificial Sequence
  Synthetic oligonucleotide
ttcattgtctggataactatacaacctgaaaactgtcatttcaggttctgtgctcttttt

SEQ ID NO: 432 - DNA, 60 bp, Artificial Sequence
  Synthetic oligonucleotide
tttgctagccaaaaggtatgggggcttcatgaaaaggtatggaggcttcatgaagaaaat

SEQ ID NO: 433 - DNA, 60 bp, Artificial Sequence
  Synthetic oligonucleotide
catttcaggttctgtgctctttttggagtccttaagctcagtattggtctgttgcagcta

SEQ ID NO: 434 - DNA, 60 bp, Artificial Sequence
  Synthetic oligonucleotide
gagaaaagcaaagctctttcttattttcctcataatcagctaccctggaggggagggaga

SEQ ID NO: 435 - DNA, 60 bp, Artificial Sequence
  Synthetic oligonucleotide
ttacctgaaccagtgaacttacaagcaagtgtgactgtttcctgtgacctgaagatagcc

SEQ ID NO: 436 - DNA, 60 bp, Artificial Sequence
  Synthetic oligonucleotide
ctgctgcctgtctccctgacccatgatctggcaagttaggcacagtcagacatggacagt

SEQ ID NO: 437 - DNA, 60 bp, Artificial Sequence
  Synthetic oligonucleotide
ggagagtggtcactggggaaaaggacctggccatcaccttccagtacctgctgcctgtct

SEQ ID NO: 438 - DNA, 60 bp, Artificial Sequence
  Synthetic oligonucleotide
ctatgaggcaccacgtaagacctcctgcccttagctctcttgctcaccacccaagaacct

SEQ ID NO: 439 - DNA, 60 bp, Artificial Sequence
  Synthetic oligonucleotide
agaatctgacatcatgacaacaaatggtgtaattcatgttgtagataaactcctctatcc

SEQ ID NO: 440 - DNA, 60 bp, Artificial Sequence
  Synthetic oligonucleotide
aggaagttgcaagccaacaaaaaagttcaaggatctagaagacgattaagggaaggtcgt

SEQ ID NO: 441 - DNA, 60 bp, Artificial Sequence
  Synthetic oligonucleotide
ttgagcccttgccgggccttttttccacctgccaattctacatgtattgttgtggtttta

SEQ ID NO: 442 - DNA, 60 bp, Artificial Sequence
  Synthetic oligonucleotide
tctccaggcagtagatcctcttcaagatcctgctattactatgagtcaagccactacaga

SEQ ID NO: 443 - DNA, 60 bp, Artificial Sequence
  Synthetic oligonucleotide
aatcactgtagtctaagacctgatctatagatgacctagaatagccatgtactataatgt

SEQ ID NO: 444 - DNA, 60 bp, Artificial Sequence
  Synthetic oligonucleotide
ttttttaaatgtgcagtacacatcagcctcactgagctaataaagggaaacgaatgtttc

SEQ ID NO: 445 - DNA, 60 bp, Artificial Sequence
  Synthetic oligonucleotide
tctcaatcactactcttcttgaagcactattatttattcttccgctgtctgcctgcagca

SEQ ID NO: 446 - DNA, 60 bp, Artificial Sequence
  Synthetic oligonucleotide
actgatcaacagcatggaccagaatatgttcaggaacttttccttcatgaaccccgggat

SEQ ID NO: 447 - DNA, 60 bp, Artificial Sequence
  Synthetic oligonucleotide
tggggtgtttgttcccattggatgcatttctatcaaaactctatcaaatgtgatggctag

SEQ ID NO: 448 - DNA, 60 bp, Artificial Sequence
  Synthetic oligonucleotide
tcaaatgtgatggctagattctaacatattgccatgtgtggagtgtgctgaacacacacc

SEQ ID NO: 449 - DNA, 60 bp, Artificial Sequence; identical to SEQ ID NO: 448
  Synthetic oligonucleotide

SEQ ID NO: 450 - DNA, 60 bp, Artificial Sequence
  Synthetic oligonucleotide
agcagcacaggaatcttacttcttggcagctgcagtctgtcaagatgagacatcagatta

SEQ ID NO: 451 - DNA, 60 bp, Artificial Sequence
  Synthetic oligonucleotide
ccagaacaaattttaacaaaaggacaaccacagagggatatagtgaatatcgtatcattg

SEQ ID NO: 452 - DNA, 60 bp, Artificial Sequence
  Synthetic oligonucleotide
tatacgaagctggaatttgatgaagcaaggagcttctggaataaaggaaattattcaaga

SEQ ID NO: 453 - DNA, 60 bp, Artificial Sequence
  Synthetic oligonucleotide
tggtactgtgatgcatttcaagtggcagttttatcacgtttgaatctaccattcatagcc

SEQ ID NO: 454 - DNA, 60 bp, Artificial Sequence
  Synthetic oligonucleotide
atttgatttccggacatatgattcagaaggcgtgatactgtacgcagaatctatcgatca

SEQ ID NO: 455 - DNA, 60 bp, Artificial Sequence
  Synthetic oligonucleotide
cagtgaagccactaaccctggagctagtggaggaaactgtgcaggctatggaggtggagt

SEQ ID NO: 456 - DNA, 60 bp, Artificial Sequence
  Synthetic oligonucleotide
acactgagctcacccacagagcccgtgaagaggtctggccgctaccactttgtgcctgga

SEQ ID NO: 457 - DNA, 60 bp, Artificial Sequence
  Synthetic oligonucleotide
ccagctgcctattgatttaagctttcctgttgaatgacaaagtatgtggttttgtaattt

SEQ ID NO: 458 - DNA, 60 bp, Artificial Sequence
  Synthetic oligonucleotide
gaacatggtttgactcatcttatatgggaaaccatgtagcagtgagtcatatcttaatat

SEQ ID NO: 459 - DNA, 60 bp, Artificial Sequence
  Synthetic oligonucleotide
tcttgtgaatggactgtcagctgttaaactgttcctgttttgaagtgctattacctttct

SEQ ID NO: 460 - DNA, 60 bp, Artificial Sequence
  Synthetic oligonucleotide
tgatcattttttggcaattaatcagaagaagagtgggaagccggtattcatttataagca

SEQ ID NO: 461 - DNA, 60 bp, Artificial Sequence
  Synthetic oligonucleotide
agaccaaaaaatgtaaccgattcattgagcacctacattaatgctaattatattaggggc

SEQ ID NO: 462 - DNA, 60 bp, Artificial Sequence
  Synthetic oligonucleotide
gttttaactacttgtctctcttttgctaagaagggatttttgaatatgctatctacctgg

SEQ ID NO: 463 - DNA, 60 bp, Artificial Sequence
  Synthetic oligonucleotide
actcaagctactggcacataatgaaagattacttcatgacattccattgctcttcttttg

SEQ ID NO: 464 - DNA, 60 bp, Artificial Sequence
  Synthetic oligonucleotide
caagctactggcacatagtgaaagattacttctgacattccattgctcttcttttgaaaa

SEQ ID NO: 465 - DNA, 60 bp, Artificial Sequence
  Synthetic oligonucleotide
aaattttggattgtatgttcaggagaagagggatggattgaaaagaaggcagcagctaga

SEQ ID NO: 466 - DNA, 60 bp, Artificial Sequence
  Synthetic oligonucleotide
tctgggtctcaggacagtgatgttggctagcccaggggaatgtatttttcaaaacataca

SEQ ID NO: 467 - DNA, 60 bp, Artificial Sequence
  Synthetic oligonucleotide
taagacatgaaaggttggccttactgttgaacaagaagtaaatccacaggctcctgttat

SEQ ID NO: 468 - DNA, 60 bp, Artificial Sequence
  Synthetic oligonucleotide
tgagactcatctgggatttggctttccttggaagctcttacgtgatgtgggaaatgacaa

SEQ ID NO: 469 - DNA, 60 bp, Artificial Sequence
  Synthetic oligonucleotide
aggcgttctctagatcctttcctctgtttccctctctcgctggcaaaagtatgatctaat

SEQ ID NO: 470 - DNA, 60 bp, Artificial Sequence
  Synthetic oligonucleotide
gatggtacatgacttgattcaacgtttggttctgaacttacacactgatgcgtttactca

SEQ ID NO: 471 - DNA, 60 bp, Artificial Sequence
  Synthetic oligonucleotide
ggttagatgccaccatgtagggattatcgcgagtggttgaccttacacttactccttaaa

SEQ ID NO: 472 - DNA, 60 bp, Artificial Sequence
  Synthetic oligonucleotide
gcttttctgtaataagcttccttttataatagtgctcagcttagctctctcagatcctat

SEQ ID NO: 473 - DNA, 60 bp, Artificial Sequence
  Synthetic oligonucleotide
tgcacggtttacgccacaaaagtgctcttgacatccgtgacaccgttttgactttttgtt

SEQ ID NO: 474 - DNA, 60 bp, Artificial Sequence
  Synthetic oligonucleotide
gggctactgccactgtgtgccttccgccaacacctcctgtccccacctaagcctggtggg

SEQ ID NO: 475 - DNA, 60 bp, Artificial Sequence
  Synthetic oligonucleotide
tagctgccttcgcaccttgctgtgtgacctgaggccctcactgagcctcaatttcctcat

SEQ ID NO: 476 - DNA, 60 bp, Artificial Sequence
  Synthetic oligonucleotide
gtgggggtgaggggctccgggtcactgctgtatataactcccctcccccagaaaaataaa

SEQ ID NO: 477 - DNA, 60 bp, Artificial Sequence
  Synthetic oligonucleotide
ctcacagtggcctgtaacaatttcttctgggagaacagttgagcagacagccacattggg

SEQ ID NO: 478 - DNA, 60 bp, Artificial Sequence
  Synthetic oligonucleotide
ataagcagcccaggaagaaatgaaaactcctctgatgtggttggggggtctgccagctgg

SEQ ID NO: 479 - DNA, 60 bp, Artificial Sequence
  Synthetic oligonucleotide
cttccacaagtactcgggcaaagagggtgacaagttcaagctcaacaagtcagaactaaa

SEQ ID NO: 480 - DNA, 60 bp, Artificial Sequence
  Synthetic oligonucleotide
ccaaaagtgtttgttggcaattattcccctaggctgagcctgctcatgtacctctgatta

SEQ ID NO: 481 - DNA, 60 bp, Artificial Sequence
  Synthetic oligonucleotide
cttaaccaaacggatgaaactctgagcaatgttgaggtgtttatgcaattaatatatgac

SEQ ID NO: 482 - DNA, 60 bp, Artificial Sequence; identical to SEQ ID NO: 481
  Synthetic oligonucleotide

SEQ ID NO: 483 - DNA, 60 bp, Artificial Sequence
  Synthetic oligonucleotide
aacataaaagctaggagatgtggcatctgaacatttttgctttgctgccagagtaaccct

SEQ ID NO: 484 - DNA, 60 bp, Artificial Sequence
  Synthetic oligonucleotide
ggtcatgtgtgctagttcaccagagaaaattgaaatcttggctcctccaaatgggtctgt

SEQ ID NO: 485 - DNA, 60 bp, Artificial Sequence
  Synthetic oligonucleotide
aacaactgtcagttcatcctgcatgggaaaaatgttggaatgggagtctgaaatggggct

SEQ ID NO: 486 - DNA, 60 bp, Artificial Sequence
  Synthetic oligonucleotide
gtatgctggtagctagtgatttacacaggtttagttgactaatgaggcattacaaataat

SEQ ID NO: 487 - DNA, 60 bp, Artificial Sequence
  Synthetic oligonucleotide
catgctctccaaaccactttttgcagctttctctagttcaagttcaccagactctataaa

SEQ ID NO: 488 - DNA, 60 bp, Artificial Sequence
  Synthetic oligonucleotide
gacaacatttgatcccaagaaaaccagaatggaaccctttcacttcaaaaactcagttat

SEQ ID NO: 489 - DNA, 60 bp, Artificial Sequence
  Synthetic oligonucleotide
tgtgtgcatactctagaagagtagggaaaataatgcttgttacaattcgacctaatatgt

SEQ ID NO: 490 - DNA, 60 bp, Artificial Sequence
  Synthetic oligonucleotide
tgaccactggagagtctaagccatccttagcatgggcatccatcttcctaaactgttatt

SEQ ID NO: 491 - DNA, 60 bp, Artificial Sequence
  Synthetic oligonucleotide
gtagtgcttatacctatatagtccaaaggaagaatgacagctgccctgaagtgaaggtgt

SEQ ID NO: 492 - DNA, 60 bp, Artificial Sequence
  Synthetic oligonucleotide
ataccctgcagtgcactgtagaacagaagataagaacttttctctgaatttgggtcttct

SEQ ID NO: 493 - DNA, 60 bp, Artificial Sequence
  Synthetic oligonucleotide
tatgtatgatgtgatctggtccagccagggcctggtttgtcagctatctaggtttgataa

SEQ ID NO: 494 - DNA, 60 bp, Artificial Sequence
  Synthetic oligonucleotide
gagatatgtagaaagactctttggttcacattccgatattaaaatagtgacatgaactgg

SEQ ID NO: 495 - DNA, 60 bp, Artificial Sequence
  Synthetic oligonucleotide
aatagtttttgttaaaccttttgtaaagtaccaaggctcccattaacaaattacggcctc

SEQ ID NO: 496 - DNA, 60 bp, Artificial Sequence
  Synthetic oligonucleotide
actacattttaaagggaatgtgtatgtgaagagcactaccaacatcgcttttgttttaag

SEQ ID NO: 497 - DNA, 60 bp, Artificial Sequence
  Synthetic oligonucleotide
agatagcagaagagtaaataagtactcagtattgaccacctacatctgaaatctacaaca

SEQ ID NO: 498 - DNA, 60 bp, Artificial Sequence
  Synthetic oligonucleotide
gaaatccaaaatagtcatgtttctgcagtattctgtagccaacttaaacctgtgctttca

SEQ ID NO: 499 - DNA, 60 bp, Artificial Sequence
  Synthetic oligonucleotide
cattgtgaatccctttgccttggaatccacaatccttgacaacgaagactcagacaccaa

SEQ ID NO: 500 - DNA, 60 bp, Artificial Sequence
  Synthetic oligonucleotide
ttggggatgattcttaccttggtaattaaatgaagctacacatttgggtaatctagcaaa

SEQ ID NO: 501 - DNA, 60 bp, Artificial Sequence
  Synthetic oligonucleotide
gtacctaagtaaggatgatctaggataagtaactcctgttttatattgagtactttaggg

SEQ ID NO: 502 - DNA, 60 bp, Artificial Sequence
  Synthetic oligonucleotide
gtgaacatttcaaccagccttatagctgttctcatcatcaccttctgcattgtgaccgtg

SEQ ID NO: 503 - DNA, 60 bp, Artificial Sequence
  Synthetic oligonucleotide
caaggcaaatgattttgtgtttcttgatgacagactattaagtttgggacttattttccc

SEQ ID NO: 504 - DNA, 60 bp, Artificial Sequence
  Synthetic oligonucleotide
aggtcctttccaccctgagacttggctccaccactgatatcctcctttggggaaaggctt

SEQ ID NO: 505 - DNA, 60 bp, Artificial Sequence
  Synthetic oligonucleotide
acagcaggatttcaggaagtgccagttgatcaatgaataaataaatgagcctatttctct

SEQ ID NO: 506 - DNA, 60 bp, Artificial Sequence
  Synthetic oligonucleotide
aagagatgttgtcctgacacttgtggcatcaaatgcctggatcctgttgacaccccaaac

SEQ ID NO: 507 - DNA, 60 bp, Artificial Sequence
  Synthetic oligonucleotide
tttaagagcgatcctcatcccttcagcaatatgtatttgagttcacactatttctgtttt

SEQ ID NO: 508 - DNA, 60 bp, Artificial Sequence
  Synthetic oligonucleotide
aagagtggcagaggctactacaaaaagcaaccttttcattttcactaagagtttaaaagc

SEQ ID NO: 509 - DNA, 60 bp, Artificial Sequence
  Synthetic oligonucleotide
gaagataatcgatagtcatgttttttagactctctgtattgcttggtaagctacgtagta

SEQ ID NO: 510 - DNA, 60 bp, Artificial Sequence
  Synthetic oligonucleotide
ttgatgtgtgggagcacgcttactaccttcagtataaaaatgtcaggcctgattatctaa

SEQ ID NO: 511 - DNA, 60 bp, Artificial Sequence
  Synthetic oligonucleotide
cgtctgggatgaaggcttgtcagcacttccagtttagaacgcaatgtttctagagacata

SEQ ID NO: 512 - DNA, 60 bp, Artificial Sequence
  Synthetic oligonucleotide
ctctggagaaagctctggaggcctttgaaacatttaaaaagggattggggttgaaaatca

SEQ ID NO: 513 - DNA, 60 bp, Artificial Sequence
  Synthetic oligonucleotide
gtagccatgacatagcttgagctatagcctttaattccttactttggctatgggtggagg

SEQ ID NO: 514 - DNA, 60 bp, Artificial Sequence
  Synthetic oligonucleotide
cagcacttccagtttagaacgcaatgtttctagagacatattggctgtttgttttgatga

SEQ ID NO: 515 - DNA, 60 bp, Artificial Sequence
  Synthetic oligonucleotide
atgagggctgagttatgaaaagataacttctgaagacttaactggcccagaagctgattt

SEQ ID NO: 516 - DNA, 60 bp, Artificial Sequence
  Synthetic oligonucleotide
accctatttccaagttcaagttaactagctttgaatgtgtcccaaaacagcttcctccat

SEQ ID NO: 517 - DNA, 60 bp, Artificial Sequence
  Synthetic oligonucleotide
gtcccaaaacagcttcctccatttcctgaaagtttattgatcaaagaaatgttgtcccgg

SEQ ID NO: 518 - DNA, 60 bp, Artificial Sequence
  Synthetic oligonucleotide
ctgggcgcgcctccgctcgacggctacccgttgcccacgcccgacacgtccccgctggac

SEQ ID NO: 519 - DNA, 60 bp, Artificial Sequence
  Synthetic oligonucleotide
ccgccatgaactacgacaagctgagccgctccatccgccagtattacaagaagggcatca

SEQ ID NO: 520 - DNA, 60 bp, Artificial Sequence
  Synthetic oligonucleotide
ttccacagccatgaatttcacagccatgaagatatgctggttgtagaccccaaaagtaag

SEQ ID NO: 521 - DNA, 60 bp, Artificial Sequence
  Synthetic oligonucleotide
gaatgtaataagaatctggtggtgtcaattgcttacttgttttcccacagttgtccagca

SEQ ID NO: 522 - DNA, 60 bp, Artificial Sequence
  Synthetic oligonucleotide
gaaatatagtccaagctttctctgtggaaaaagacaaaactcattagtagacatgtttcc

SEQ ID NO: 523 - DNA, 60 bp, Artificial Sequence
  Synthetic oligonucleotide
tcattagtagacatgtttccctattgctttcataggcaccagtcagaataaagaatcata

SEQ ID NO: 524 - DNA, 60 bp, Artificial Sequence
  Synthetic oligonucleotide
atatccccttggatttcacttgcattgtgcaataagcaaagaagggttgataaaagttct

SEQ ID NO: 525 - DNA, 60 bp, Artificial Sequence
  Synthetic oligonucleotide
ctggaagagaacaccattttatctcaggttagtgaagaatcagtgcaggtccctgactct

SEQ ID NO: 526 - DNA, 60 bp, Artificial Sequence
  Synthetic oligonucleotide
aattaaagaggtaccctttgagacactagcccagtggaatctagaacacgctactttaaa

SEQ ID NO: 527 - DNA, 60 bp, Artificial Sequence
  Synthetic oligonucleotide
agggaactaatgcaactggaaaaggagctggtagaacgtcaacctcaagtggacatgtta

SEQ ID NO: 528 - DNA, 60 bp, Artificial Sequence
  Synthetic oligonucleotide
aacctcaagtggacatgttacaggagatttcaaacagccttctcattaagggacatggag

SEQ ID NO: 529 - DNA, 60 bp, Artificial Sequence
  Synthetic oligonucleotide
aatctgggaggcagaagccaaatctgttttggatcaagatgatgtggacacctcaatgga

SEQ ID NO: 530 - DNA, 60 bp, Artificial Sequence
  Synthetic oligonucleotide
caatgcgccacctatgagtctgtcaatgtgaccgattttaagtcaagttggagaaatggg

SEQ ID NO: 531 - DNA, 60 bp, Artificial Sequence
  Synthetic oligonucleotide
aagaagctaaagagaaagtccagatcaatgtggtaaaactcattgcagcgttgaagaact

SEQ ID NO: 532 - DNA, 60 bp, Artificial Sequence
  Synthetic oligonucleotide
agctccacgggaagaacctggaactggggaagatcatggacaggttcgaagaggttgtgt

SEQ ID NO: 533 - DNA, 60 bp, Artificial Sequence
  Synthetic oligonucleotide
ttgaagaagaaccagcccagcctgcctcctatcttttcctggaatatttttggggttgga

SEQ ID NO: 534 - DNA, 60 bp, Artificial Sequence
  Synthetic oligonucleotide
acccgtgtggtaccttcagccctggccaagctttgaggctctgtcactgagcaatggtaa

SEQ ID NO: 535 - DNA, 60 bp, Artificial Sequence
  Synthetic oligonucleotide
ccgtggagatcccaactggtttatgaagaaagcgcaggagcataagagggaattcacaga

SEQ ID NO: 536 - DNA, 60 bp, Artificial Sequence
  Synthetic oligonucleotide
caagcaagagcactgcctctatagggtaacctggaacattctctaggttatatcaatata

SEQ ID NO: 537 - DNA, 60 bp, Artificial Sequence
  Synthetic oligonucleotide
gccggaagctgatgaacatcgccttcaatgacatgaaccccttccgcatgaaacagctgc

SEQ ID NO: 538 - DNA, 60 bp, Artificial Sequence
  Synthetic oligonucleotide
ccagaaaatgaatgatactatatttggtttcacaatggaggagcgctcatgggggcccta

SEQ ID NO: 539 - DNA, 60 bp, Artificial Sequence
  Synthetic oligonucleotide
tccgcccttccctgacactgtctgctgccccaatcgccgtcacaataaaagaaagtgtgg

SEQ ID NO: 540 - DNA, 60 bp, Artificial Sequence
  Synthetic oligonucleotide
tcacaggctctccgtggcctggaactgcagccccagctgcatcctacacccccaccccaa

SEQ ID NO: 541 - DNA, 60 bp, Artificial Sequence
  Synthetic oligonucleotide
ctgtccacgctgtactggttcacggtggagttcgggctgtgtaagcagaacggggaggtg

SEQ ID NO: 542 - DNA, 60 bp, Artificial Sequence
  Synthetic oligonucleotide
ggatgaggaaattgagaagctgtccacgctgtcatggttcacggtggagttcgggctgtg

SEQ ID NO: 543 - DNA, 60 bp, Artificial Sequence
  Synthetic oligonucleotide
ttccagactgcttccaatttttctggaacacattaaatatggatcagttataagtagcag

SEQ ID NO: 544 - DNA, 60 bp, Artificial Sequence
  Synthetic oligonucleotide
acagggtctgcaaggtctttggttcagctaagctaggaatgaaatcctgcttcagtgtat

SEQ ID NO: 545 - DNA, 60 bp, Artificial Sequence
  Synthetic oligonucleotide
tctttctgtaaccataacaacttcatatatgaggacttgtgtctctgtgcttttaaatgc

SEQ ID NO: 546 - DNA, 60 bp, Artificial Sequence
  Synthetic oligonucleotide
actttttacttttttgcgtgtggagctgtattcccgagaccaacgaagcgttgggatact

SEQ ID NO: 547 - DNA, 60 bp, Artificial Sequence
  Synthetic oligonucleotide
tcgttgtgttgttttgctgcactttttacttttttgcgtgtggagctgtattcccgagac

SEQ ID NO: 548 - DNA, 60 bp, Artificial Sequence
  Synthetic oligonucleotide
tgtggagtgaaaattgggcatgccattacattgctttttcttggtggttaaaaagaatga

SEQ ID NO: 549 - DNA, 60 bp, Artificial Sequence
  Synthetic oligonucleotide
caagatggcacccaagtgtttggcttctggctacctaaggttaacatgtcactagagtat

SEQ ID NO: 550 - DNA, 60 bp, Artificial Sequence
  Synthetic oligonucleotide
ctgtctagaaggaatgtatttgttgctaaattttgtagcactgtttacagttttcctcca

SEQ ID NO: 551 - DNA, 60 bp, Artificial Sequence
  Synthetic oligonucleotide
ccttttatggaaaccgttttttaaaaaagtgaatgtacacaaatccacagaagactgtgg

SEQ ID NO: 552 - DNA, 60 bp, Artificial Sequence
  Synthetic oligonucleotide
atgagtgatctaaatttgcagcaatgatactaaacaactctctgaaatttctcaagcacc

SEQ ID NO: 553 - DNA, 60 bp, Artificial Sequence
  Synthetic oligonucleotide
cttatctggtgtgttcatatagaatcacctagaaggataaagtcgctgtagagttaatga

SEQ ID NO: 554 - DNA, 60 bp, Artificial Sequence
  Synthetic oligonucleotide
tcagggtttcaagaagtcttagggcttccaggggtcccctggaagctttagaatatttat

SEQ ID NO: 555 - DNA, 60 bp, Artificial Sequence
  Synthetic oligonucleotide
gaagaaactgcttgttgtgtatcagtaatcattagtggcaatgatgacattctgaaaagc

SEQ ID NO: 556 - DNA, 60 bp, Artificial Sequence
  Synthetic oligonucleotide
aagaaactgcttgttgtgtatcagtaatcattagtggcaatgatgacattctgaaaagct

SEQ ID NO: 557 - DNA, 60 bp, Artificial Sequence
  Synthetic oligonucleotide
gccgggctggggctgcgtaggtgaaaaggcagaacactccgcgcttcttagaagaggagt

SEQ ID NO: 558 - DNA, 60 bp, Artificial Sequence
  Synthetic oligonucleotide
tgtagttcttgcatcctatactggataagcctgttttaacctgctatgatgggtgcttcc

SEQ ID NO: 559 - DNA, 60 bp, Artificial Sequence
  Synthetic oligonucleotide
tagagattttaggcgtcttcggatatcttctcacctatgttccctggctaagaagtcaga

SEQ ID NO: 560 - DNA, 60 bp, Artificial Sequence
  Synthetic oligonucleotide
tgtgaggcgattattttaagtaattatcttaccaagcccaagactggttttaaagttacc

SEQ ID NO: 561 - DNA, 60 bp, Artificial Sequence
  Synthetic oligonucleotide
ctcacctttgggacaggcactcagctagaagtgggactcaatatccagaaccctgaccct

SEQ ID NO: 562 - DNA, 60 bp, Artificial Sequence
  Synthetic oligonucleotide
atcttcagtgggttctcttgggctctaggtcctggagaatgttgtgaggggtttattttt

SEQ ID NO: 563 - DNA, 60 bp, Artificial Sequence
  Synthetic oligonucleotide
tttctttaaccatttttgaaacccttcaaaggcagagacttgtccagcctaacctgcctg

SEQ ID NO: 564 - DNA, 60 bp, Artificial Sequence
  Synthetic oligonucleotide
atcattcatggtctaaaaaacaatgaaaccaatgaaatggcctctctgatcatcacagaa

SEQ ID NO: 565 - DNA, 60 bp, Artificial Sequence
  Synthetic oligonucleotide
tagtgttcataaagaaatacatagtattcttcttctcaagacgtggggggaaattatctc

SEQ ID NO: 566 - DNA, 60 bp, Artificial Sequence
  Synthetic oligonucleotide
tctggctgtgggataaatgtgtgtgggaatattgaaacatcgcctaggaattgtggtttg

SEQ ID NO: 567 - DNA, 60 bp, Artificial Sequence
  Synthetic oligonucleotide
agcccctttcccttgtcaatgacagtcatcctaatgataataaaacctgcatccagataa

SEQ ID NO: 568 - DNA, 60 bp, Artificial Sequence
  Synthetic oligonucleotide
gctgattggggaaataattttcaacactatcctgaattatgtgcctgtctagataagcag

SEQ ID NO: 569 - DNA, 60 bp, Artificial Sequence
  Synthetic oligonucleotide
ttgagatactgggtttggtgttttctatggtcctgtattgccagatcgggaacaaatgaa

SEQ ID NO: 570 - DNA, 60 bp, Artificial Sequence
  Synthetic oligonucleotide
tgatgactctgcttctatgaggtcaccagcagtagaaccatatcttgctggcatacactt

SEQ ID NO: 571 - DNA, 60 bp, Artificial Sequence
  Synthetic oligonucleotide
atccaacttatccaaaaatcagaaaggatatacttgataaggcccgtgcgtctttaagac

SEQ ID NO: 572 - DNA, 60 bp, Artificial Sequence
  Synthetic oligonucleotide
aaagccaacatgaaggggtcatatagtcttgtagaagcacagaaatcaaaagtgtagcta

SEQ ID NO: 573 - DNA, 60 bp, Artificial Sequence
  Synthetic oligonucleotide
ctttactgcgaataagcttttaatgctccaaatgctgacccatgcaatatttcctcatgt

SEQ ID NO: 574 - DNA, 60 bp, Artificial Sequence
  Synthetic oligonucleotide
agtttatactcaccttttatgaaagcactgcatgaataaaattattcctttgtattttta

SEQ ID NO: 575 - DNA, 60 bp, Artificial Sequence
  Synthetic oligonucleotide
cactcttagagtccagcttgtaatggttctttacacatgagtcacaagttacagctgtga

SEQ ID NO: 576 - DNA, 60 bp, Artificial Sequence
  Synthetic oligonucleotide
tcactgcatacattgtggaaggtgtagggagtgaagtctcacataggaggacctgtgtga

SEQ ID NO: 577 - DNA, 60 bp, Artificial Sequence
  Synthetic oligonucleotide
catgaataaagttattcctttgtatttttacttttaaatgtcttctgcattcacttatat

SEQ ID NO: 578 - DNA, 60 bp, Artificial Sequence
  Synthetic oligonucleotide
ccagttagcagaatcaagacctacaccatcacggaaggctccttgagagcagtaattttt

SEQ ID NO: 579 - DNA, 60 bp, Artificial Sequence
  Synthetic oligonucleotide
ctcatttcactttacaccctcatggactgagattatactcaccttttatgaaagcactgc

(19) United States
(12) Patent Application Publication
(10) Pub. No.: US 2019/0276890 A1
(43) Pub. Date: Sep. 12, 2019

(54) GENE EXPRESSION PROFILE AS AN ENDOMETRIAL RECEPTIVITY MARKER

(71) Applicant: IGENOMIX S.L., Valencia (ES)

(72) Inventors: Carlos Simón Vallés, Valencia (ES); José Horcajadas Almansa, Valencia (ES); Patricia Diaz Gimeno, Valencia (ES); Antonio Pellicer Martínez, Valencia (ES)

(73) Assignee: IGENOMIX S.L., Valencia (ES)

(21) Appl. No.: 16/140,437

(22) Filed: Sep. 24, 2018

Related U.S. Application Data
(63) Continuation of application No. 13/057,135, filed on Mar. 11, 2011, now Pat. No. 10,081,840.

(51) Int. Cl. C12Q 1/6883

(52) U.S. Cl. CPC C12Q 1/6883; C12Q 2600/112; C12Q 2600/158

(57) ABSTRACT

The present invention relates to determining the receptivity of human endometrium from a gene expression profile. More specifically, the invention consists of developing a specific expression microarray of endometrial receptivity (Endometrial Receptivity Array or ERA) which allows evaluating the receptive state of a human endometrium, as well as assessing said state for diagnostic and therapeutic purposes.

### FIELD OF THE ART

The present invention relates to determining the receptivity of the human endometrium from a gene expression profile. More specifically, it consists of developing a specific expression microarray of endometrial receptivity (Endometrial Receptivity Array or ERA) which allows evaluating the receptive state of a human endometrium, as well as assessing said state for diagnostic and therapeutic purposes.

### PRIOR ART

The endometrium is the mucosa coating the inside of the uterine cavity. Its function is to house the embryo, allowing its implantation and favoring the development of the placenta. This process requires a receptive endometrium capable of responding to the signals of the blastocyst, which is the stage of development of the embryo when it implants. Human endometrium is a tissue cyclically regulated by hormones, the hormones preparing it to reach said receptivity state are estradiol, which induces cell proliferation, and progesterone which is involved in differentiation, causing a large number of changes in the gene expression profile of the endometrium, which reaches a receptive phenotype for a short time period referred to as “window of implantation”. Though there is no consensus as to the implantation period in humans, clinical studies suggest that this process takes place between days 20 and 24 of a normal ovulation cycle (Wilcox et al., 1999), day LH+7 (day 20-21) being considered critical.

The evolution of our knowledge about the human endometrium contrasts with the lack of progress in developing new diagnostic methods for the dating and study thereof. The endometrium is still evaluated today by means of histological studies based on in observations described over 50 years ago (Noyes et al., 1950) or with macroscopic techniques with little resolution as equally non-objective ultrasound studies which lack specificity and produce widely varying results.

In 1950, Noyes et al. described for the first time a method for endometrial dating based exclusively on histological criteria and on the morphological changes of the different compartments of the endometrium in response to the presence of estrogens and progesterone. Noyes et al. studied the histological features of endometrial biopsies taken during 8,000 spontaneous cycles in 300 women (Noyes et al., 1950). They were able to relate different histological patterns with particular moments of the menstrual cycle by correlating the histological changes with the basal body temperature. These morphological criteria continue to be used today and are considered the Gold Standard for the study of the endometrium, evaluation of endometrial receptivity and detection of endometrial anomalies.

However, this technique does have its drawbacks. It has been demonstrated that the use of histological features fails when distinguishing the phase of the menstrual cycle, and it also fails as a means to discriminate between fertile and infertile women, concluding that it is not suitable for clinical use. The subjectivity involved in visual observation means that there is an inter-observer, intra-observer and inter-cycle variability altering the consistency of the results obtained. Furthermore, ovarian stimulation typical of assisted reproductive treatments (ART) modifies the endometrial maturation process compared to natural cycles which can barely be explained with Noyes' criteria (Papanikolaou et al., 2005). For this reason there are many works in the literature which question the histological observations interpreted by one or several pathologies both in retrospective clinical studies (Balash et al., 1992; Batista et al., 1993; Shoupe et al., 1989), prospective clinical studies (Li et al., 1989; Creus et al., 2002; Ordi et al., 2003), and recently in randomized studies (Murray et al., 2004; Coutifaris at al., 2004). The Practice Committee of the American Society for Reproductive Medicine (ASRM) also establishes that even though the classic criterion of the luteal phase defect consists of a delay in the endometrial maturation of ≥2 days following the Noyes criteria, this Committee has serious doubts as to the accuracy of said histological criteria and therefore of the prevalence of the luteal phase defect (LPD) and even of its clinical relevance as a cause of infertility (ASRM, 2000).

In this sense, Balasch et al., 1992 demonstrated that the incidence of LPD and histological endometrial patterns were similar in fertile and infertile women. Moreover, a suitable endometrial histology in the ovulation cycle or in previous ones was riot related to the pregnancy data in infertile women concluding that the histological evaluation of the endometrium in the luteal phase is not useful for predicting or improving the reproductive results (Balasch et al., 1992). In other studies of the same group, it was demonstrated that there was a clear dissociation in the temporary expression of a series of markers related to the window of implantation (alpha and beta 3 integrins) and the pinopod expression. They furthermore did not find differences in the expression of these markers between fertile and infertile women (Crews et al., 2002). They also demonstrated a high variability between cycles and low reproducibility for these markers (Ordi et al., 2003).

Li et al. 1989 dated 63 endometrial biopsies on two different occasions by the same pathologist, demonstrating that there was complete agreement in only 24% of them. In a separate study, they observed that between different cycles in the same woman, there was complete agreement in only 4% of the cases. These data emphasize the lack of precision of traditional dating methods and their lack of any assurances for predicting the development in the following cycles (Li et al., 1989).

The differences between pathologists varied depending on the day of the menstrual cycle in which the endometrial biopsy is taken. Over 20% of the endometrial biopsies were dated with a difference of at least two days between pathologists in the early, mid and late luteal phases. Inter-cycle variations reach 60% in the mid luteal phase (Murray et al., 2004). It has been demonstrated that during the window of implantation, a very similar percentage of women has the endometrium out of phase, 49.4% fertile versus 43.2% infertile (p=0.33) and, ultimately, that the histological dating is not related to fertility status (Coutifaris et al., 2004). These variations described suggest that the traditional criteria are not precise and that new technologies are required for dating and functionally identifying the endometrial samples.

In the pre-genomic era, only “gene-by-gene” studies could be carried out to select useful candidates for studying uterine receptivity or for determining the endometrial situation in women with or without endometriosis.

Therefore, in the present genomic era objective tools based on molecular criteria which improve the diagnostic capacity of determined techniques such as the histological technique, which is very useful, however, for other types of needs, are sought.

In the mid 1990s (Schena et al., 1995), a revolutionary technology was developed for determining and quantifying the expression of messenger RNA (mRNA) in a sample, gene expression microarrays. Their main advantage is that they offer the possibility of simultaneously analyzing thousands of genes in a single experiment. A DNA microarray consists of a large number of DNA molecules arranged on a solid substrate such that they form an array of sequences in two or three dimensions. These fragments of genetic material can be short sequences called oligonucleotides or larger sequences, such as complementary DNA (cDNA) which is synthesized from mRNA, or PCR products (in vitro replication of DNA sequences by means of the polymerase chain reaction). These single-strand nucleotides fragments immobilized on the support are referred to as “probes”. The nucleic acids of the samples to be analyzed are labeled using different methods (enzymatic, fluorescent methods, etc.) and are incubated on the probe panel, which allows hybridization (recognition and binding between complementary molecules) of homologous sequences. During hybridization, the labeled genetic material samples bind to their complementary samples immobilized on the support of the chip, allowing the identification and quantification of the DNA present in the sample. The suitable bioinformatic tools and scanner then allow interpreting and analyzing the data obtained (Al-Shahrour F et al., 2005).

To use a microarray, commercially available microarrays can be used or one can be custom designed.

To design a microarray, the following operations must be performed:

a) Choosing the type of probe, oligos, cDNA, . . .

b) Labeling probes or samples: enzymatic, fluorescent, . . .

c) Support material: glass, plastic, membranes, . . .

d) Immobilizing probes: active, passive, covalent, . . .

e) Manufacturing: printing, in situ synthesis, . . .

f) Detecting hybridization: scanner, fluorometry, . . .

g) Data processing: software.

This technology is being applied to the analysis of gene expression, sequencing, therapy follow-up, preventive medicine, drug toxicology and molecular diagnosis. The manufacture of microarrays, also referred to as bioarrays or biochips has been described in various patent documents, such as for example WO 2005/018796 A1, US 2005/0048554 A1, and US 2005/0046758 A1. Their use has also been applied to dendrimers (WO 2005/040094 A1) and large biomolecules (US 2005/0042363 A1) or for collecting information on samples, such as for example identifying a carcinogenic or pathogenic cell in an individual (WO 2005/016230 A2). Their use is also known for immobilizing nucleic acids which are complementary to a variety of genes, being applied to the field of chemistry, biology, medicine and medical diagnostics (U.S. Pat. No. 6,821,724 B1). Microarrays are currently being used to make comparisons based on genomic data and to research different systems.

There are different patent and non-patent literature publications on this subject. Microarray technology has allowed globally studying the gene expression of the endometrium under physiological conditions during the different phases of the menstrual cycle in the natural cycle (Ponnampalam et al., 2004, Talbi et al., 2005). With respect to the human window of implantation, gene expression profiles of the endometrium in the natural cycle have been described (Borthwick et al., 2003; Carson et al., 2002; Riesewijk et al., 2003; Mirkin et al., 2005). The gene expression profile of the endometrium during the window of implantation in stimulated cycles has also been analyzed (Mirkin et al., 2004; Horcajadas et al., 2005 (Provide literature reference in the Literature section); Simón C et al., 2005) and in response to drugs such as RU486 (Catalano et al., 2003 (Provide literature reference in the Literature section); Sharkey et al., 2005).

The refractory profile of the human endometrium in the presence of an intrauterine device (IUD) during the window of implantation has also been studied (Horcajadas et al. 2006). All these works have recently been reviewed by the authors of the present application (Horcajadas et al., 2007). The conclusion of said study is that even though different genomic studies of the human endometrium in different physiological and pathological conditions have been conducted in the last 4 years, generating a large amount of information on the gene regulation during the window of implantation both in fertile and infertile women, the key molecules and mechanisms have yet to be discovered.

In the field of patents, there are several which try to determine endometrial receptivity/non-receptivity, though neither the genes, nor the technology, nor the predictive systems they postulate coincide with those used in the present invention.

Patent document US 2003/0077589 A1 describes a method for diagnosing endometriosis based on identifying the product of at least one of the genes of the group consisting of fibronectin, PTK7 transmembrane receptor, type XVIII collagen, alpha 1, protein similar to subtilisin (PACE4), laminin M chain (merosin), elastin, type IV collagen, alpha 2, interferon-alpha-inducible gene p27, reticulocalbin, aldehyde dehydrogenase 6, gravin, nidogen and phospholipase C epsilon, in which a small amount of the control gene indicates the presence of endometriosis.

Patent application US 2003/0125282 A1 describes two human MATER proteins (mice MATER proteins were already known) and their relationship and use for fertility disorders.

Document US 2003/0186300 A1 describes methods and commercial compositions for the diagnosis and treatment of reproduction-associated diseases. The invention also relates to methods and compositions for the determination and modulation of endometrial receptivity.

Patent US 2005/0032111 A1 uses the expression of cadherin-11 in endometrial tissue as an indicator of the capacity for establishing or maintaining a pregnancy.

Document US 2005/0106134 A1 relates to the role of the enzyme proprotein convertase 5/6 during pregnancy, and particularly its detection and the detection of its isoforms in the uterus. This enzyme is useful in fertility control for monitoring a premature pregnancy and for detecting the uterine receptivity in mammals. New forms of proprotein convertase 5/6 are also described.

Patent US 2003/0228636 A1 describes a method for detecting endometrial receptivity for embryo implantation, which comprises: obtaining a sample of the endometrium, contacting the endometrium with a monoclonal antibody for β₃, and detecting β₃ in the endometrium. Contraceptives and diagnostic kits useful for carrying out the methods of the invention are also mentioned.

Patent application WO 2005/061725 A1 describes methods for detecting markers associated with endometrial diseases or a determined endometrial phase in a woman, which comprise measuring the peptide endometrial markers or the polynucleotides encoding the markers in the studied sample. The invention also provides methods for detecting endometrial diseases, as well as kits for carrying out the methods of the invention.

Document WO 01/39548 A2 relates to the pharmaceutical use of the fibulin-1 polypeptide and nucleic acid in birth control in women, and for the diagnosis and treatment of the endometriosis.

In patent WO 2004/058999 A2, the invention relates to a method and the means for determining the specific conditions or changes in the uterine mucosa or in the epithelium of other organs. The method allows determining the overexpression of type 1-β (β7, β6, B6e) mRNA subunits of human gonadotropin. The measurements of the expression of β7, β6, β6e are used to indicate the receptivity of the uterine mucosa to implantation of an embryo or to indicate neoplastic changes in epithelia.

Patent US 2004/0005612 A1 identifies genetic sequences with expression levels which are suppressed or induced in the human endometrium during the window of implantation. The genes characterized during the window of implantation provide material for screening tests for the purpose of determining endometrial alterations and fertility disorders, as well as endometrial-based birth control methods.

Patent U.S. Pat. No. 6,733,962 B2 describes a method for diagnosing abnormal endometrial development of a woman based on the expression of cyclin E and p27 in a sample obtained after day 20 of the menstrual cycle of a woman which ideally lasts 28 days.

In summary, for over 50 years the attempt has been made to determine a histological standard for being used in the clinical diagnosis of endometrial receptivity based on morphological observations. Today, with microarray technology, which is much more precise than morphological observations, works have been published relating to different genes present throughout the menstrual cycle, but the results do not coincide because the experimental design, collecting the samples and selecting the genes are crucial for reaching any conclusions.

Therefore, it is still and more than ever necessary to have a microarray which encompasses selecting genes which generate an expression profile that serves to diagnose and determine if the state of a particular endometrium corresponds to the receptivity/non-receptivity state.

Therefore, a list of genes and probes has been determined in this application which, once incorporated to a microarray, by means of analyzing the joint expression of these genes in the sample under study using a defined and trained computational prediction model, is capable of evaluating the receptivity/non-receptivity state of a sample of the endometrium obtained 7 days after the LH surge, as well as situations of sub-fertility of an endometrial origin depending on the gene expression profile of all of them.

Therefore, the method of the present invention uses the joint expression of the process-related mRNA as a whole as an endometrial receptivity marker, unlike the remaining receptivity molecular markers of the prior which are based on studying a molecule or a small group of molecules considered independently.

### OBJECT OF THE INVENTION

The present invention allows determining the human endometrial receptivity functional state by means of using two components: on one hand, the design of a specific microarray which identifies the gene expression profile of the situation of human endometrial receptivity/non-receptivity and on the other, the subsequent analysis of the expression profile of this specific microarray by means of a computational predictor which is capable of assigning a receptivity status.

To that end, the steps described below are followed:

1. Identifying a set of genes that are involved in endometrial receptivity for their inclusion in a specific microarray of endometrial receptivity (Endometrial Receptivity Array, ERA).

2. Creating the specific microarray.

3. Analyzing the expression pattern of the ERA during the window of implantation by means of bioinformatic tools, to be able to establish the endometrial receptivity profile and create a prediction model.

4. Developing software which, with this prediction model based on the gene expression profile, allows quantitatively and objectively evaluating and predicting the in vivo endometrial receptive state.

The foundation of the microarray is the following: when a gene is active, mRNA molecules which have a base sequence complementary to that of the gene are produced. When a gene is inactive mRNA is not produced. The analysis consists of extracting the total mRNA from two cell populations which vary in the situation to be studied, in this case receptive and non-receptive endometrium, labeling it with a fluorescent substance and hybridizing it on the microarray. Since each mRNA matches up only to the probe of the gene having the same complementary base sequence, those probes which capture the most mRNA—and which therefore shine with more fluorescence—will indicate which genes were the most active. If the fluorescence pattern of the receptive endometrium is compared to that of the non-receptive endometrium, it will be known which genes are differentially expressed in one situation with respect to the other, and that they are therefore process-related.

The probes are designed so that the mRNA of the gene to which they belong bond to them and are fixed in the support of the array. The oligonucleotides forming the probe are inserted in an automated manner in a layer of glass, nylon or plastic, being placed in squares acting like a micro-test tube. The oligonucleotide microarrays are made in an automated manner and inserted by robots by means of photolithography or piezoelectric printing. The result is an automated and normalized process which allows thousands of printings per cm² and minute.

The distribution of the probes in the microarray as a set of probes is generally observed; those having the same sequence are located at the same point in the array. In the ERA of the present invention, the probes are oligos with 60 nucleotides. Therefore, what is labeled and loose an the solution hybridized in the microarray are labeled mRNA fragments, which will bind to the probe fixed to the support as explained, by sequence homology, such that the more labeled mRNA that binds to at one point, which corresponds to the specific probe of a gene, the more light will be detected at that point and it is therefore concluded that said gene is the most active.

Having established the operation of the microarray object of the invention, and having delimited the receptivity expression pattern for evaluating the receptivity/non-receptivity state of an endometrium by means of bioinformatic methods, the receptivity states of other pathological processes resulting in infertility or subfertility of an endometrial origin, such as implantation failure due to an endometrial cause and hydrosalpinx, can also be evaluated using the same method.

In addition to the use of the microarray of the present invention for molecular diagnosis, the latter can also be used as a biotechnological tool for studying the possible effect of drugs and/or inert devices in the endometrium, such as for example the response to contraceptive drugs, both in in vitro and in vivo assays.

More specifically, the microarray of the present invention is suitable for determining from a biological sample of human endometrium the normalcy/abnormality situation in the receptive profile of said endometrium, because the microarray is a customized expression microarray which analyzes the mRNA set of the biopsy. The expression receptivity profile is defined and classified to that end and using a computational prediction model. It is also capable of defining the normal receptivity state and other situations of receptivity, both subfertility and infertility, as well as the exposure to drugs and/or inert devices, because software is used to analyze the microarray which contains the necessary information so that from an endometrial biopsy obtained during the receptive period and after being analyzed by the ERA, the gene expression data are preprocessed, such that the sample is classified in the class determined by the prediction model.

The microarray of the present invention is an oligo expression microarray with an 8×15K format (8 arrays of 15,000 probes) per slide (FIG. 3). Each array contains 15,744 points: 659 probes in which are included the selected genes (8 replicas per probe, 4552 points), 536 control points and 10656 free (empty) points.

### BRIEF DESCRIPTION OF THE FIGURES

FIG. 1 shows a list of the 569 probes corresponding to the 238 genes with an FDR<0.05 and an FC>3 which are those which have been selected and are specified in FIG. 2.

FIG. 2 shows a list of the 238 genes selected with an FDR<0.05 and an FC>3.

FIG. 3 shows a specific microarray (ERA) (Agilent Technologies). The figure shows how the ERA, oligo expression microarray, has a format of 8×15K (8 arrays with 15,000 probes) per slide. Each array contains 15,744 points: 569 probes in which the selected genes are included (8 replicas per probe, 4,552 points), 536 control points and 10656 free (empty) points.

FIG. 4 shows a table in which the forward and reverse primers designed from the genes to be amplified by means of quantitative PCR are shown.

FIG. 5 shows the mean expression of the probes of each gene in the array compared with the expression in the quantitative PCR.

FIG. 6 shows a diagram summarizing how the molecular tool and the main components which form it have been designed.

FIG. 7 shows the result of a computational prediction model generated with a training set of 23 samples having the described characteristics, which have been analyzed with the ERA. A. The prediction model distinguishes between two classes, Receptive (samples on day 20-21) and Other (samples on days of the cycle outside receptivity). The rows show each of the samples analyzed with the ERA array, and column 1 shows the actual class known a priori and column 2 shows the class assigned by the prediction model. It is observed that it predicts with a 100% success rate after calculating the error by cross-validation. B. Confusion matrix in which it is seen that 11 samples are classified as other days of the cycle and 12 samples are classified as receptive, there being no false positives or false negatives.

FIG. 8 shows a diagram of the process to be followed for determining the state of endometrial receptivity of a woman.

### DETAILED DESCRIPTION OF THE INVENTION

Endometrial receptivity is the state in which the endometrium is prepared for embryo implantation. This occurs in all menstrual cycles in a time period referred to as window of implantation, which has a variable duration and opens around day 19 of the cycle and closes on day 24, day 21 being considered a reference day.

Ovulation occurs after the luteinizing hormone (LH) surge, which occurs around day 14. A more exact way to know the actual moment in the menstrual cycle is to measure this LH surge in blood, the day it occurs being considered as day LH 0 and day 15 of the cycle LH+1 and day 21 of the cycle LH+7.

A molecular diagnostic tool allows analyzing the transcriptome of a subset of genes of the genome related to the receptivity status.

After taking an endometrial biopsy on day 21 of the menstrual cycle (receptive phase, LH+7), it can be evaluated whether the woman has a normal receptive endometrium or whether, on contrast, the expected expression pattern is not shown.

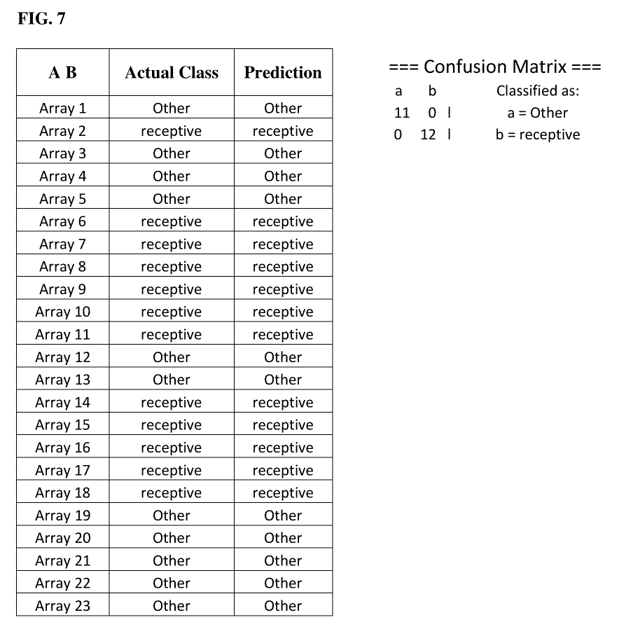

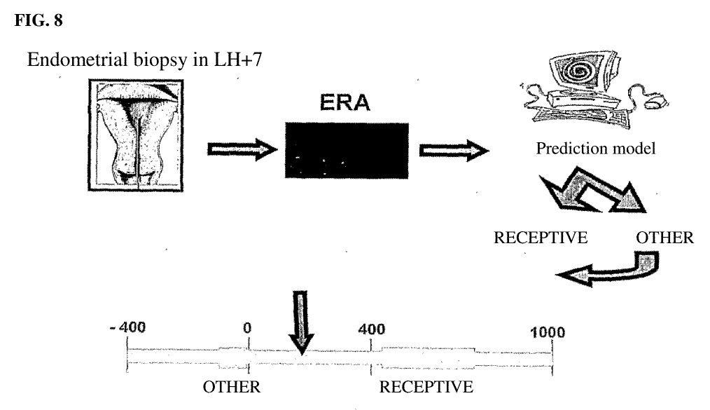

The endometrial biopsy is processed to extract its RNA, and this labeled RNA will hybridize with the probes fixed in the ERA, being able to detect the expression levels of the genes depending on the intensity of each point by means of a scanner. The data of the intensities of each point are analyzed by the prediction model which has previously been trained, and this model, depending on the entire set of points, classifies the samples as normal receptive samples or outside of normalcy samples (FIG. 8).

The prediction model is a mathematical system using different algorithms, formulas, to distinguish between classes, and is trained with the training set to define the normal receptivity profile, and to define the receptivity profile of endometrial pathologies or status of subfertility due to endometrial causes which cause implantation failure; such as endometriosis, hydrosalpinx, etc., for example, which would be the main causes.
1. Identifying the genes involved in endometrial receptivity for generating the specific microarray of endometrial receptivity.

The first phase of the project consists of identifying the genes which are specifically regulated in the endometrium of day LH+7 and which will be part of the customized microarray.

In most published works, the mentioned genes have been selected when they are induced or suppressed two times. Different and stricter selection criteria have been followed in the present invention:

### Gene Selection Criterion.

The genes have been selected based on the differences of the endometrial gene expression profile represented by LH+1, LH+3 and LH+5 (non-receptive) against LH+7 as the receptive state. The expression levels have been obtained from a whole genome oligo expression microarray. Those genes showing significant differences of expression in these two situations have been chosen using the criteria of FDR<0.05* and FC>3**.

*FDR: False Discovery Rate. This parameter corrects the P-value depending on the size of the sample. The value of FDR 0.05 is the significance that is typically taken into account at the statistical level and involves running a 5% risk that the differences are due to chance and not to the biological process in question.

**FC: Fold change. This means the number of times that the expression of a gene changes in one situation with respect to another. With regard to FC>3, the criterion is to assume that if it changes more than three times, it is sufficient change to consider the gene important for the process.

The possibility that the differences of expression may be due to chance and not to the biological process has been considered with FDR. Furthermore, the genes with an Fc above a threshold value of 3 have been selected so that the final number of genes worked with is feasible. More importance is therefore given to the genes which change the most because a directly proportional ratio between more changes and greater importance for the process is assumed. This strict criterion combines both the statistical and the biological requirement. Furthermore, the functional sense of this gene selection has been corroborated in the biological process of endometrial receptivity. To that end, the genes were ontologically classified by means of bioinformatic tools using FATIGO GEPAS (Al-Shahrour F et al., 2005) given that the biological processes represented in a manner exceeding what is expected with a significance of 0.05 are the response to stress, the defense response and cell adhesion, which are fairly relevant processes in preparing an endometrium for the possible implantation of the blastocyst.

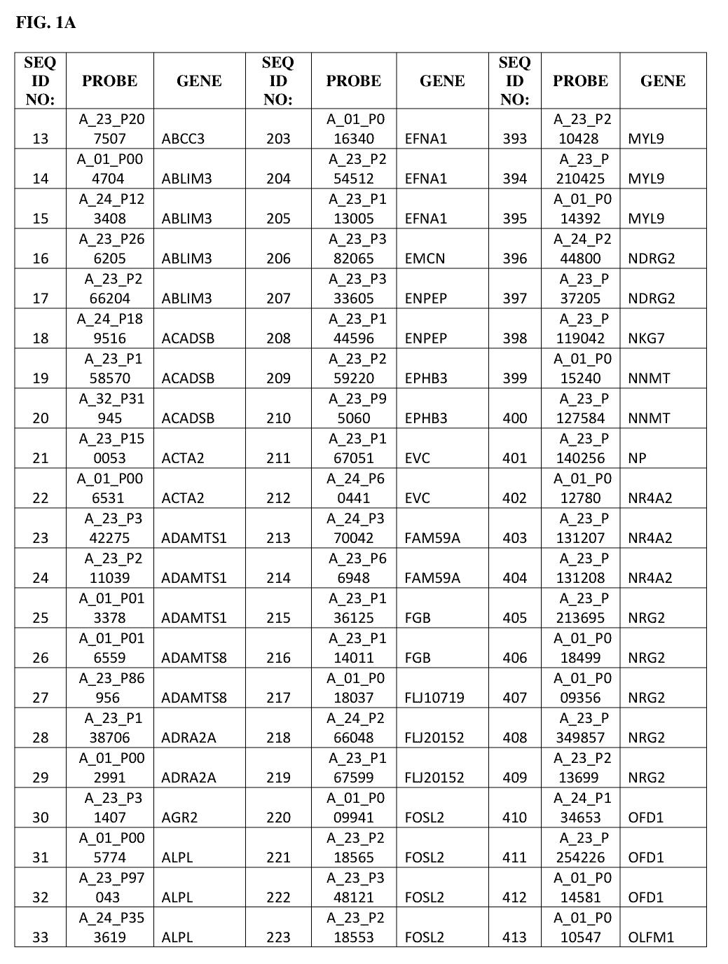

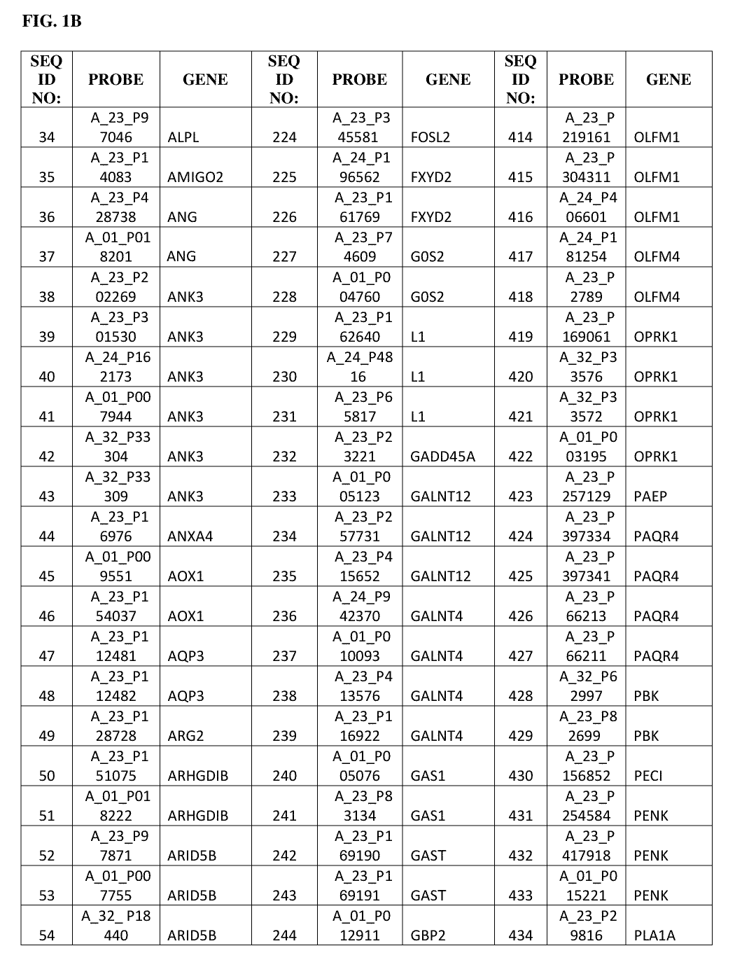

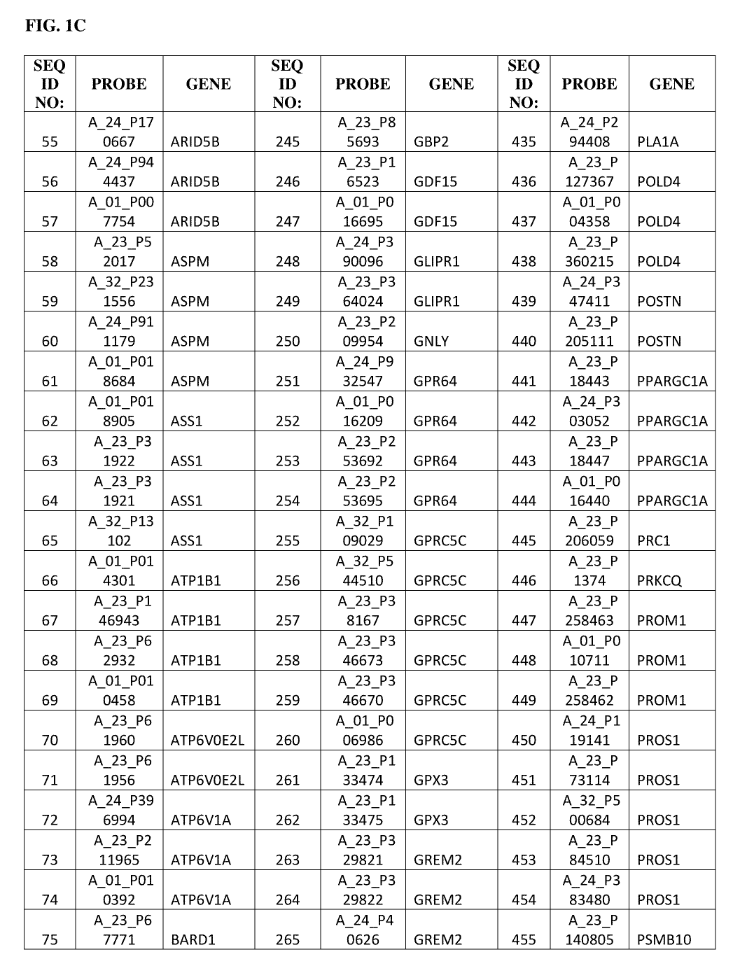

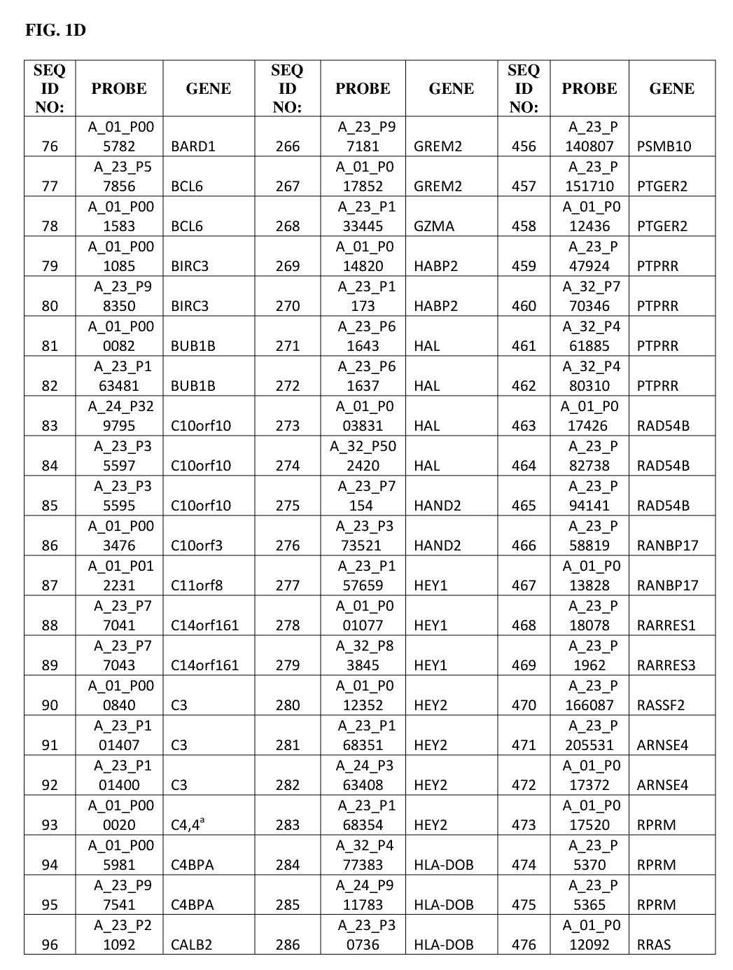

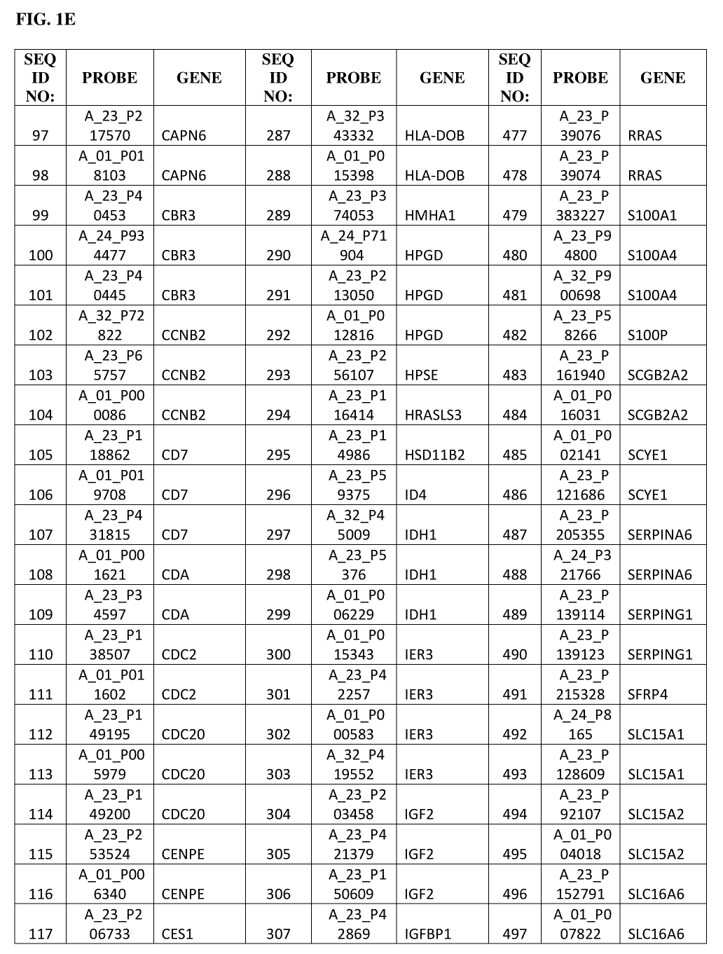

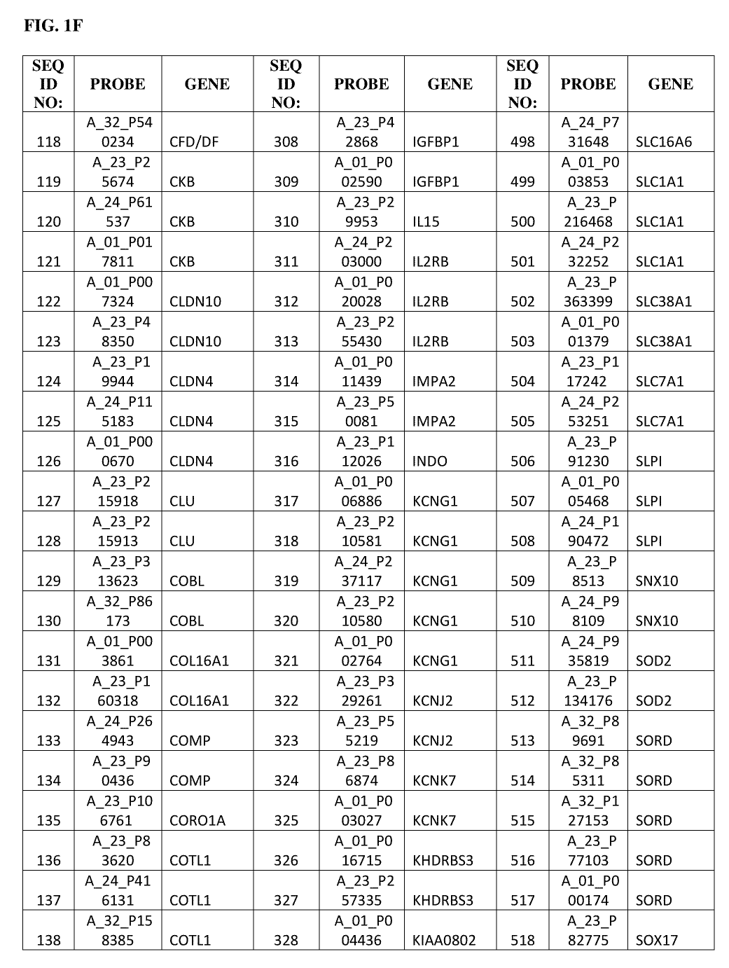

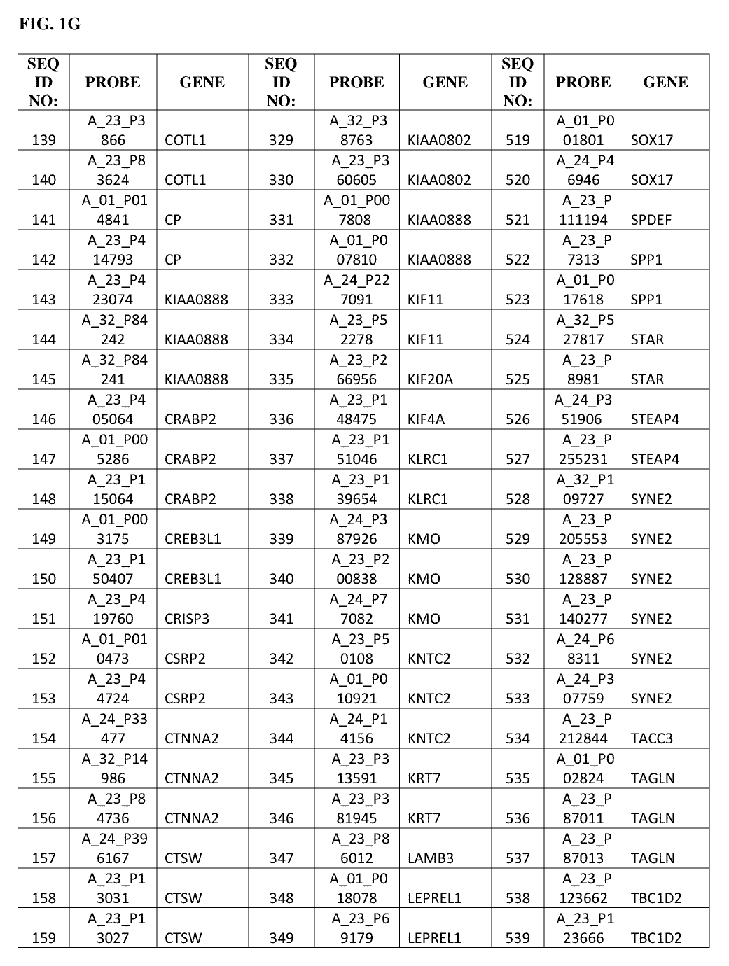

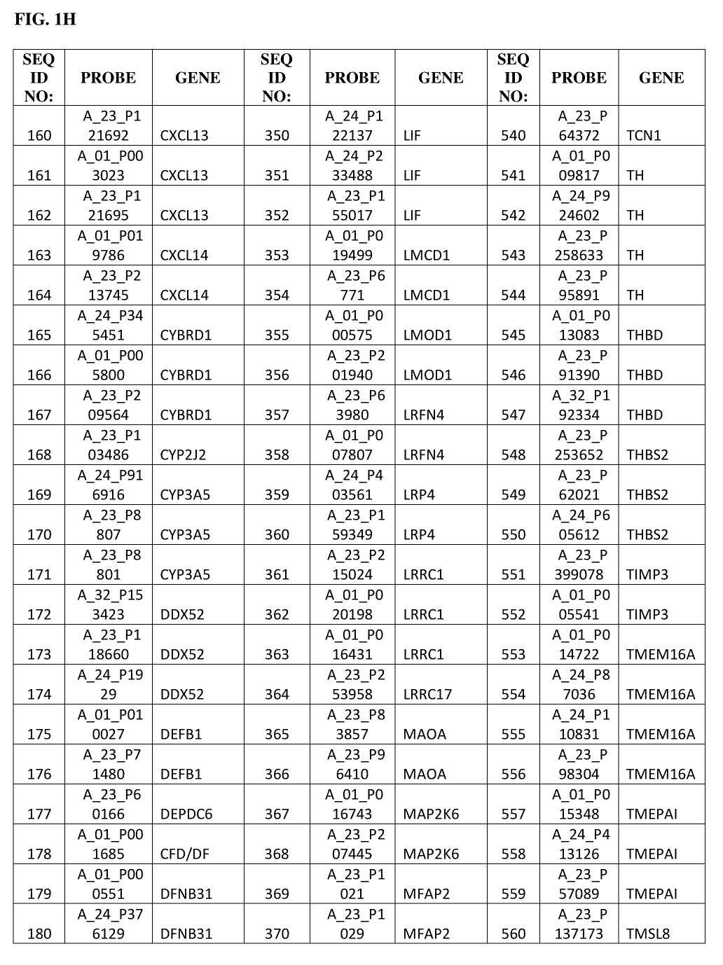

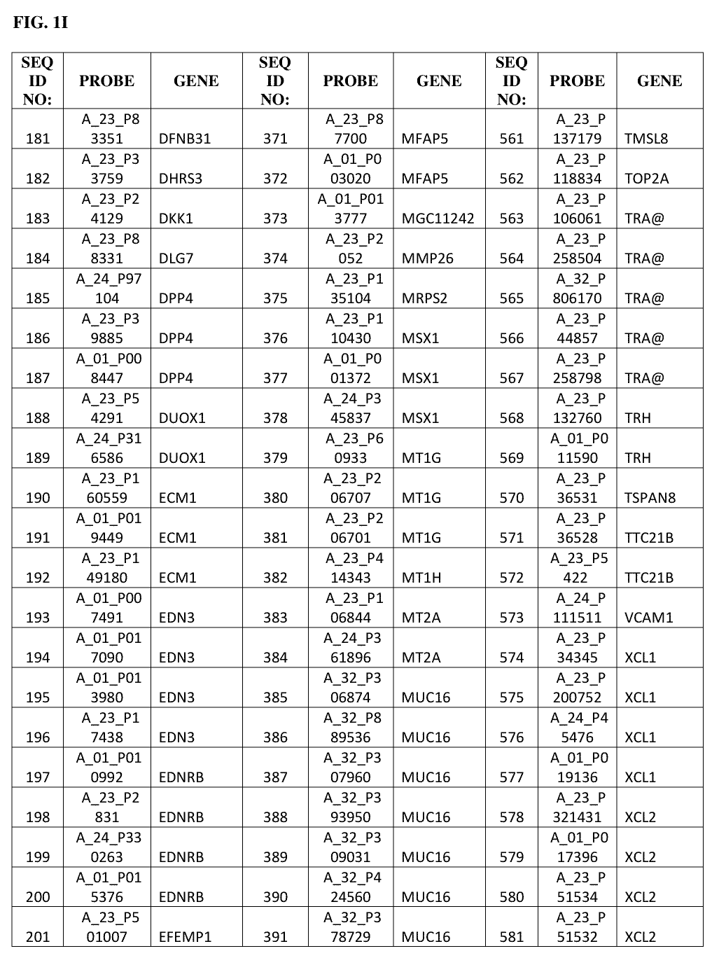

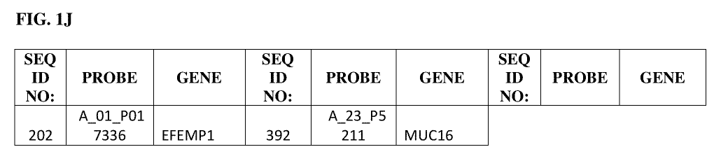

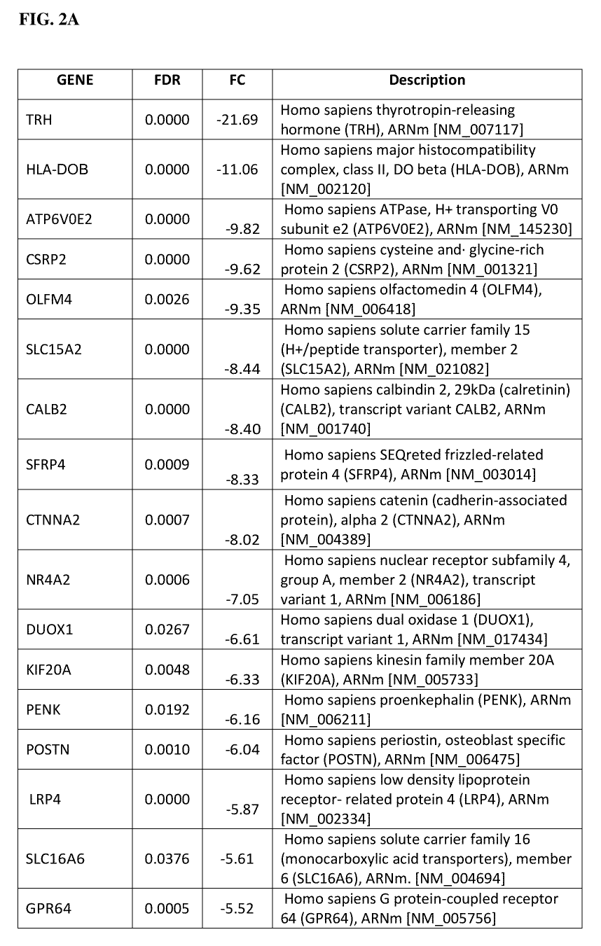

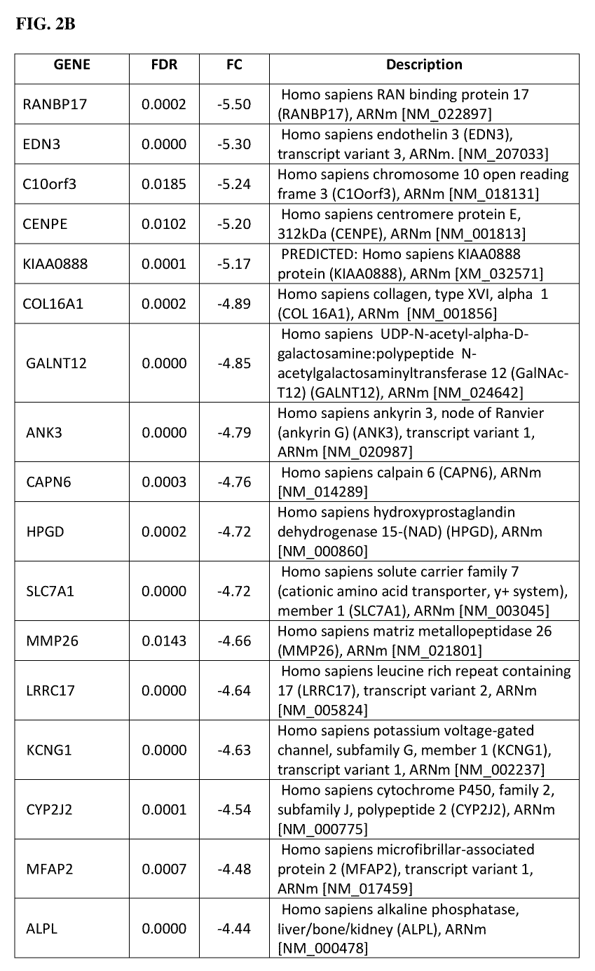

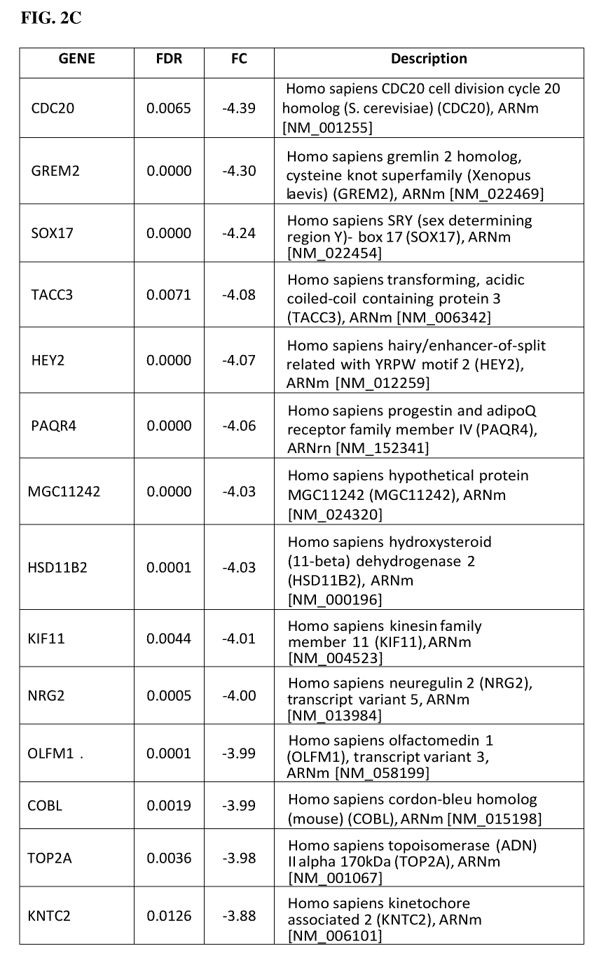

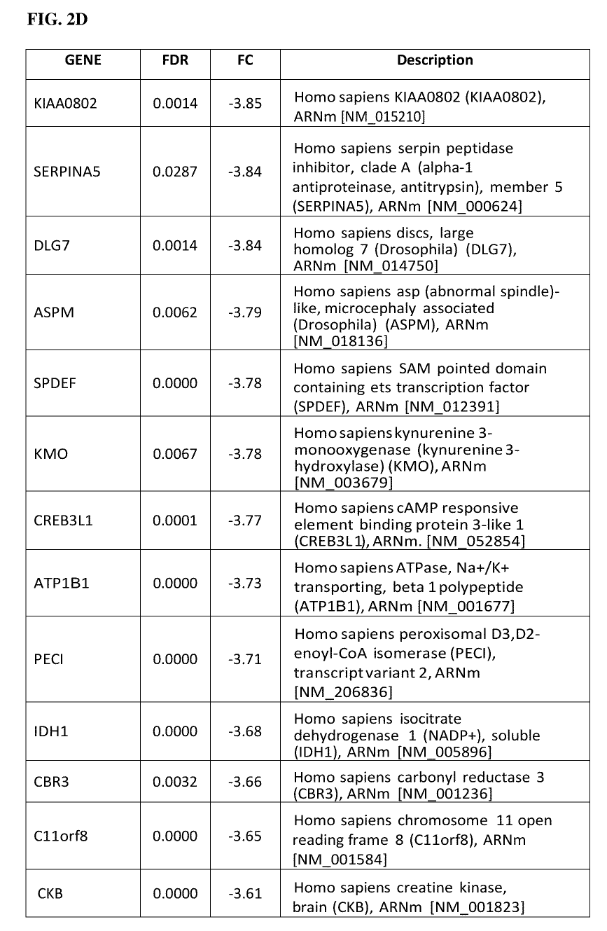

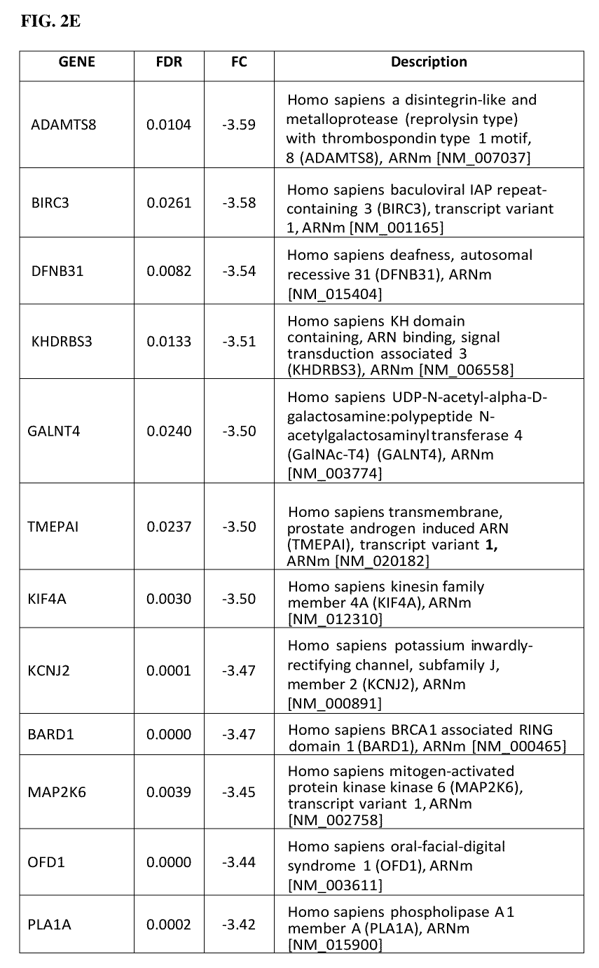

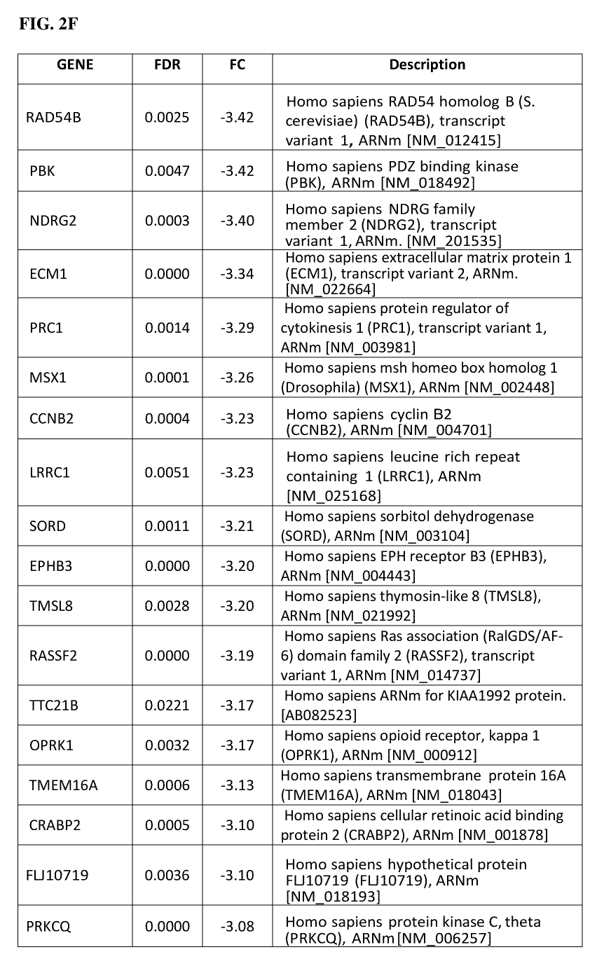

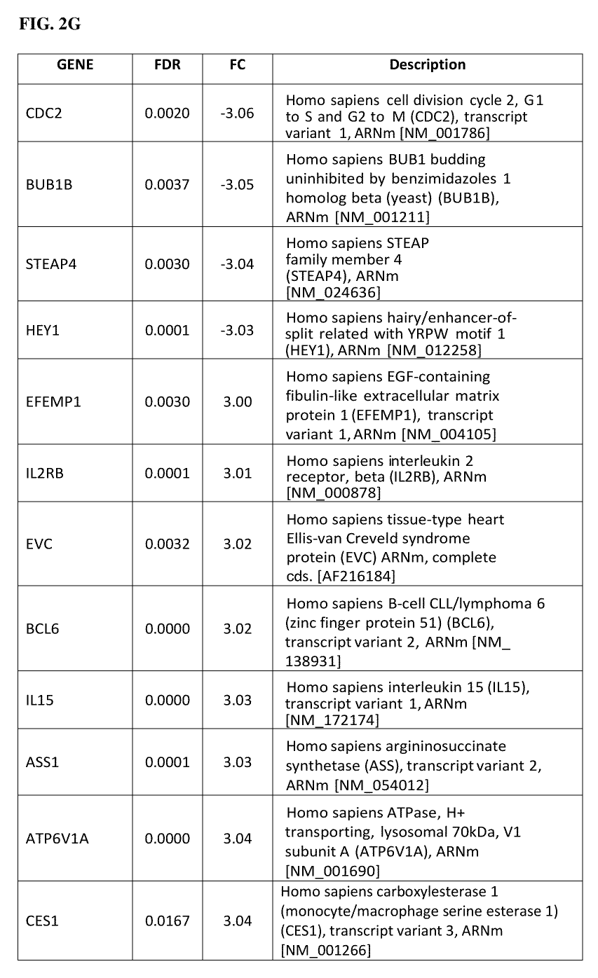

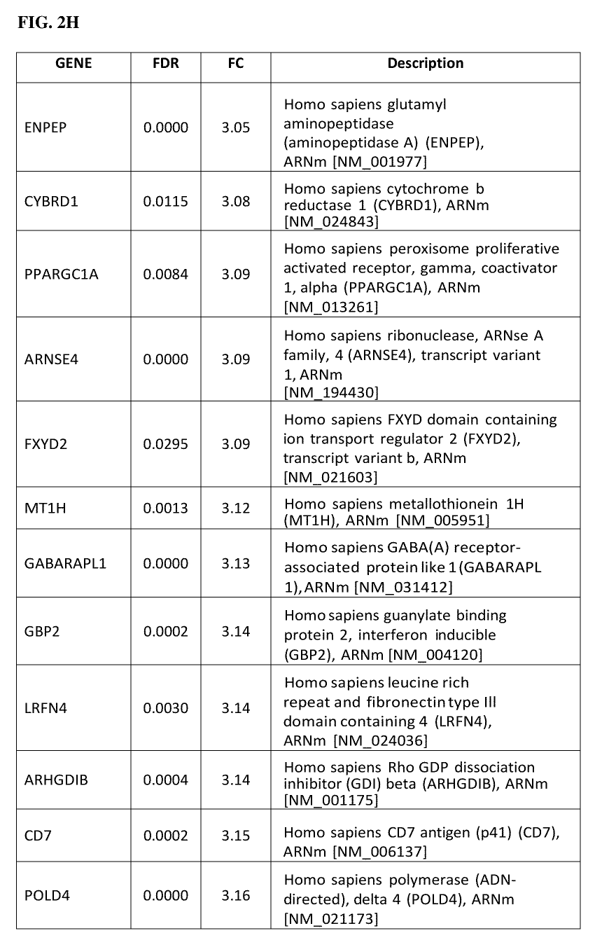

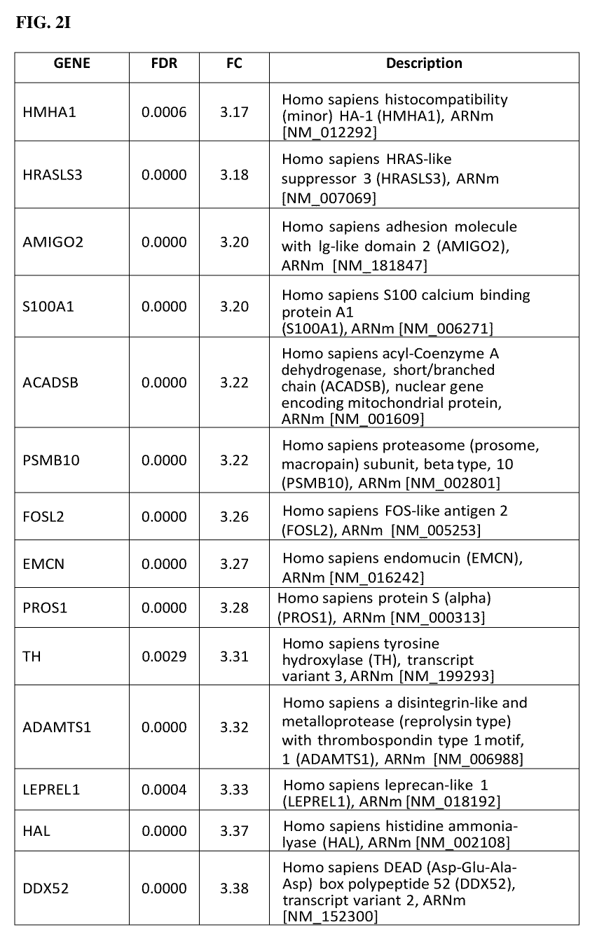

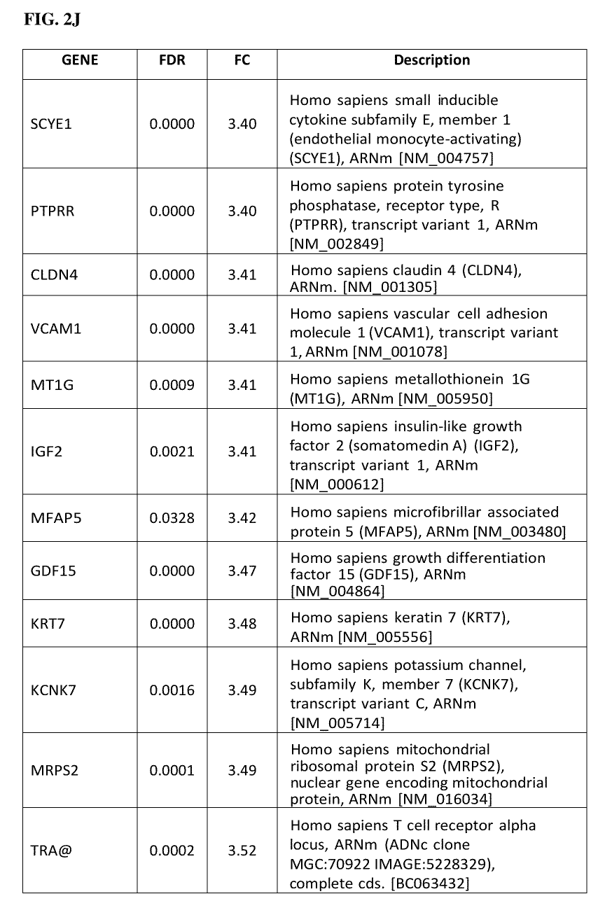

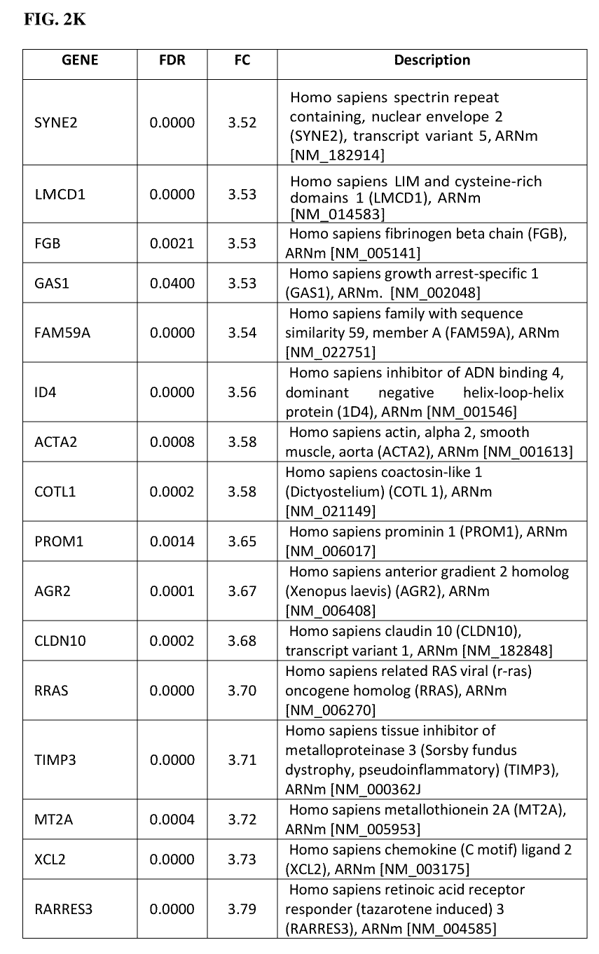

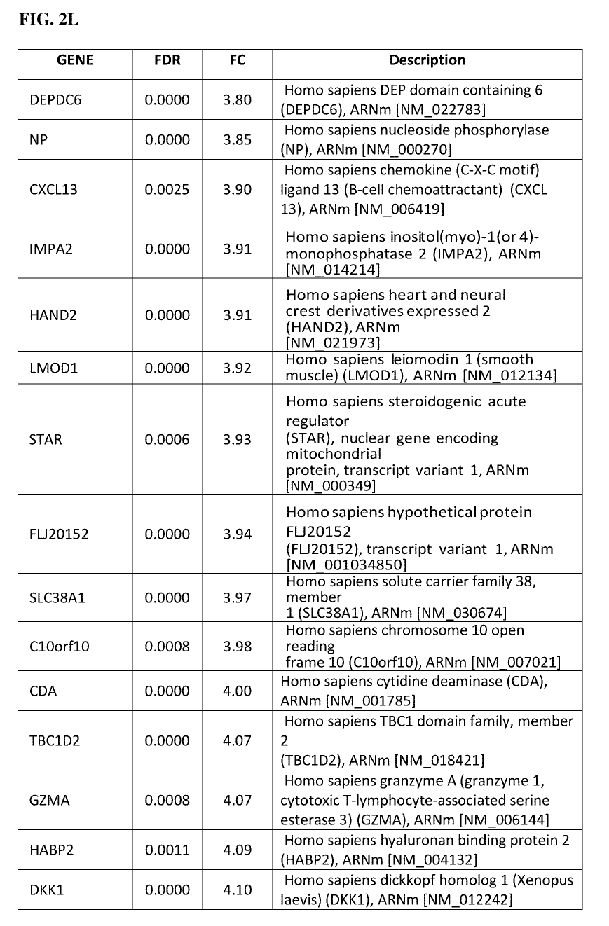

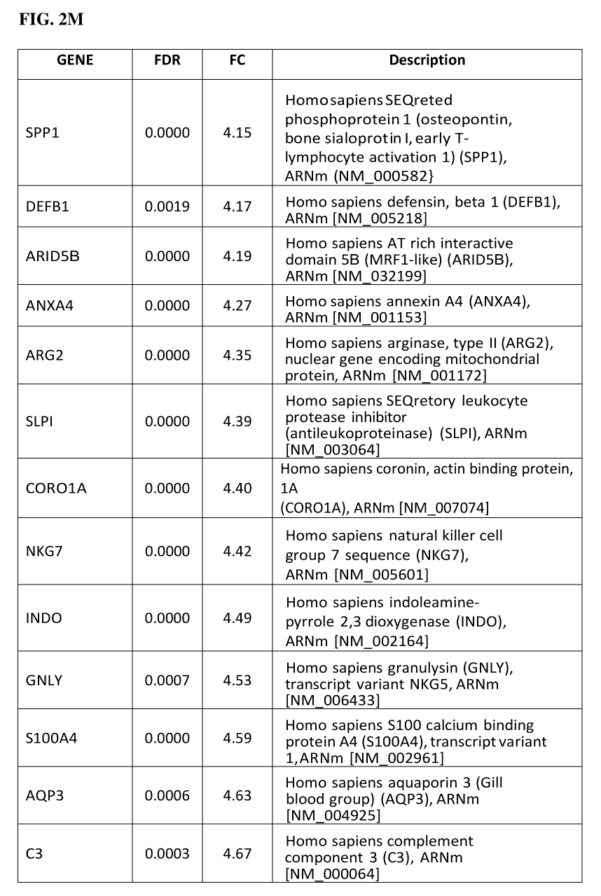

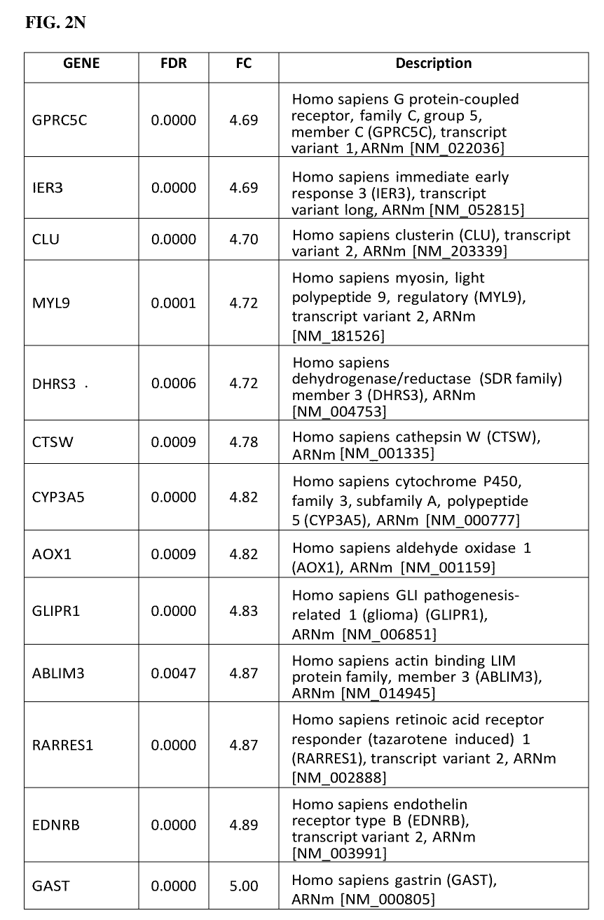

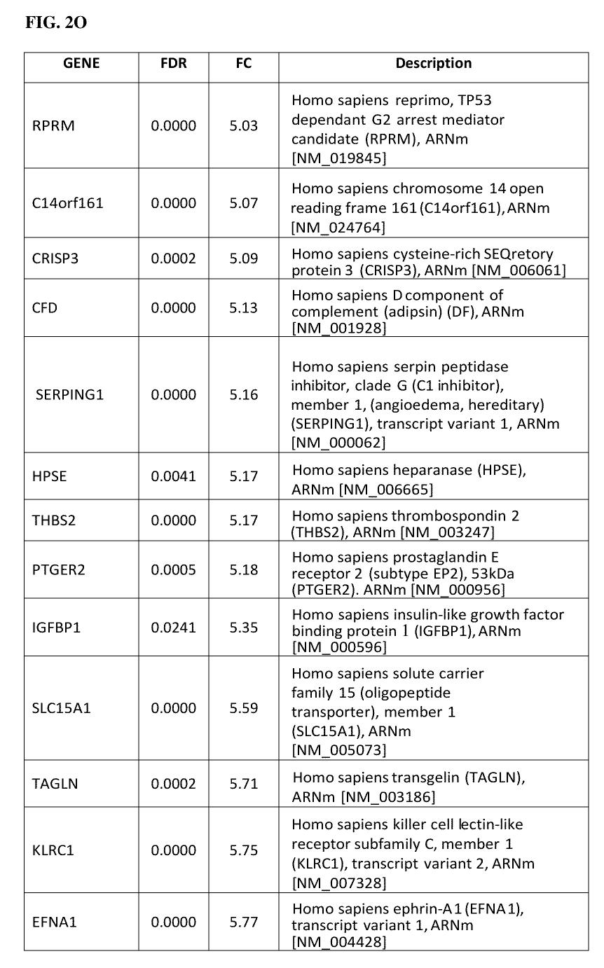

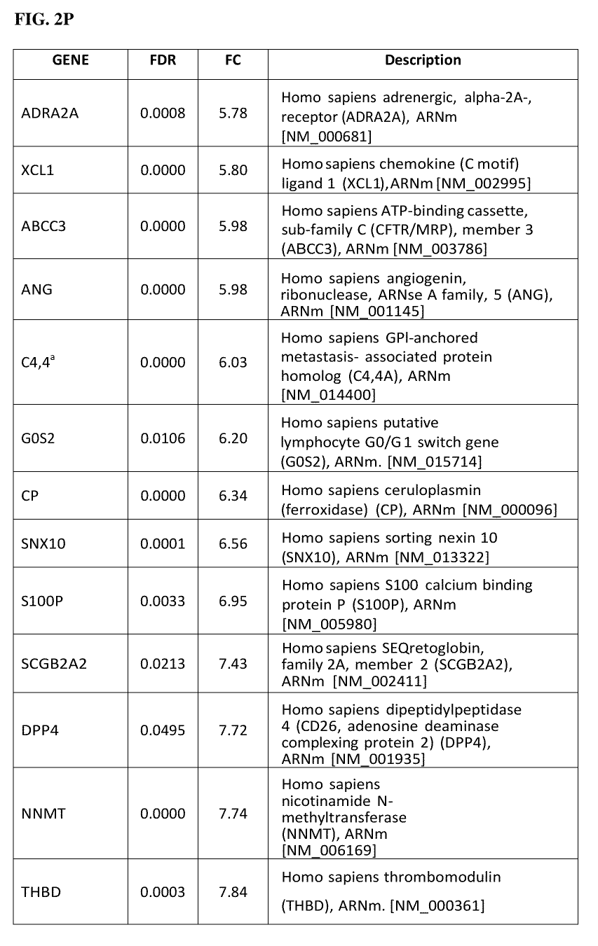

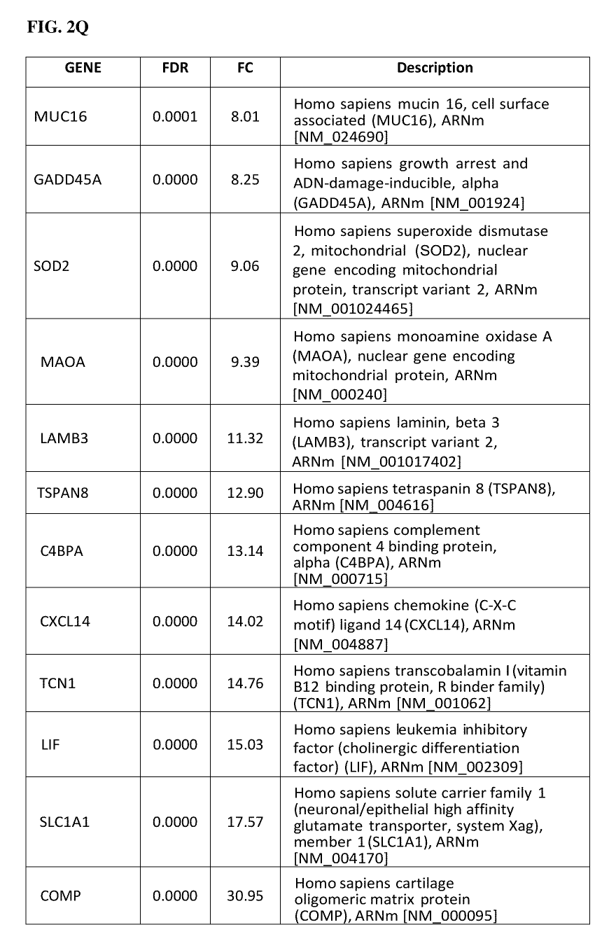

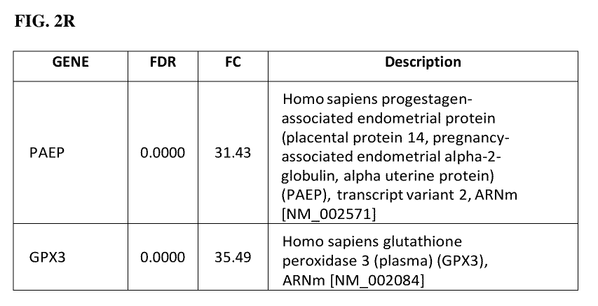

Those genes with these characteristics have been chosen and this has resulted by means of computer programs in a total of 238 genes (FIG. 2) represented by 569 probes (FIG. 1).
2. Creating the specific microarray (Era) (Agilent Technologies)

The ERA is an oligo expression microarray with a format of 8×15K (8 arrays of 15,000 probes) per slide (FIG. 3). Each array contains 15,744 points: 569 probes in which the selected genes are included (8 replicas per probe, 4,552 points), 536 control points and 10,656 free (empty) points.

### Expression Analysis by Means of the ERA

In this section, the expression data generated by the ERA for classifying the endometrial samples in two or more classes according to the different receptivity profiles that are generated (normal receptive; pathological receptive; normal non-receptive . . . ) are used to generate the prediction model and to check its efficacy.

To that end, endometrial biopsies of fertile women are selected. All the independent samples are from women with proven fertility on different days of the menstrual cycle. They are Caucasian women with a body mass index between 19 and 25 kg/m² and whose ages range between 18 and 35 years old.

Said samples were used to generate a prediction model.

To that end, the total RNA was extracted using the Trizol protocol (Invitrogen) following the manufacturer's instructions (Life Technologies, Inc., USA). The samples were homogenized using 1 ml of trizol for each 75 mg of tissue, they were incubated at room temperature for 5 minutes, and 200 μl of chloroform were added for the same amount of tissue and were incubated at room temperature for 5 minutes. They were then centrifuged for 15 minutes at 12,000×g (4° C.). The aqueous phase was precipitated with an equal volume of 2-propanol (isopropanol), it was incubated on ice for 5 minutes and centrifuged for 30 minutes at 12,000×g (4° C.). The precipitate was washed with 70% ethanol in water treated with diethylpyrocarbonate (DEPC) to subsequently resuspend it in water-treated DEPC (15 μl). This protocol usually produces 1-2 μg of total RNA per mg of endometrial tissue. The RNA thus extracted is treated with DNase for 1 hour at 37° C. to remove the traces of DNA and purify it again using the Qiagen RNeasy kit following the manufacturer's instructions. The RNA that is obtained after the columns of the RNeasy kit is analyzed to check its quality in the Agilent 2100 bioanalyzer using the Agilent brand RNA specific chips, RNA Nano LabChip.

Only those RNAs having the following characteristics have been used for subsequent analyses:

They did not have detectable genomic DNA,

They had a concentration greater than 200 μg/ml,

The value of the radius of rRNA was 28s/18S>1.2, and

The RIN (RNA Integrity Number) value>7.0.

After the analyses with the samples selected due to their suitable quality, single-stranded complementary DNA (cDNA) generated from the total RNA by incubating it between one and two hours at 40° C. with reverse transcriptase, nucleotides and an oligonucleotide polydT-T7, which has not only the poly T sequence which hybridizes with the polyA tail of messenger RNA, but also the recognition sequence for T7 RNA polymerase.

The cDNA obtained in the previous step is incubated for 2 hours at 40° C. in the presence of T7 RNA polymerase and nucleotides, one of which is labeled with Cy3, to produce complementary RNA called cRNA.

That cRNA is purified by means of a purification kit based on affinity chromatography and is quantified.

Once purified, that labeled cRNA is fragmented for 30 minutes at 60° C. and hybridized in the microarray for 17 hours 65° C. Once that time has elapsed, the microarray is washed to remove unspecific hybridizations. Once hybridized and washed, the microarrays are centrifuged at 3,000 rpm for 3 minutes to dry the microarrays and they are then read by means of scanning them in an Axon GenePix 4100A, reading for Cy3 intensities (532 nm).

As a result, after the relevant data processing enclosed below, a gene expression matrix was generated the rows of which correspond to the 569 probes of the 238 genes selected and the columns of which correspond to the different samples.

### Processing the Data of the Array

The correction of the bottom effect has been done by subtracting half the median of the latter from the intensity of the point. Interarray normalization has been done using the quantile method.

The mean of the eight replicas of each probe is then calculated. The different probes of the same gene (probe set) are analyzed individually and the results are processed by bioinformatic tools.

### Validating the Results of the ERA by Means of PCR

The results obtained in the ERA have been validated by means of quantitative PCR for the purpose of giving the results greater consistency and checking that the microarray analysis is reliable.

Reverse transcription is performed to obtain RNA in the form of cDNA, to that end 1 μg of total RNA was placed in the presence of 1 μg oligo (dT) (Clontech) until reaching a final volume of 12.5 μl with water treated with DEPC (diethylpyrocarbonate). It was heated for 2 minutes at 70° C. so that any possible secondary structure in the mRNA would denature, and it was then kept on ice for 2 minutes.

Then 6.5 μl of a MIX solution with 4 μl of buffer, 1 μl dNTP, 0.5 μl RNase and 1 μl of reverse transcriptase (Rt-PCR Clontech) were added for each of the 30 samples to be validated. The reverse transcription lasted for 1 hour in the thermal cycler. 80 μl of water with DEPC are added and concentration of single-stranded cDNA obtained is measured by spectrophotometry placing 2 μl of sample and 98 μl of DEPC-treated water. The amount of cDNA that has been reverse transcribed must be between 80 to 120 ng/μl to start from similar concentrations, though it is normalized with the internal pattern, in our case GAPDH. In any case, in order for the quantitative PCR to work correctly, the range of cDNA to be amplified must be between 50-500 ng/μl. If any sample is not within those parameters, it is diluted.

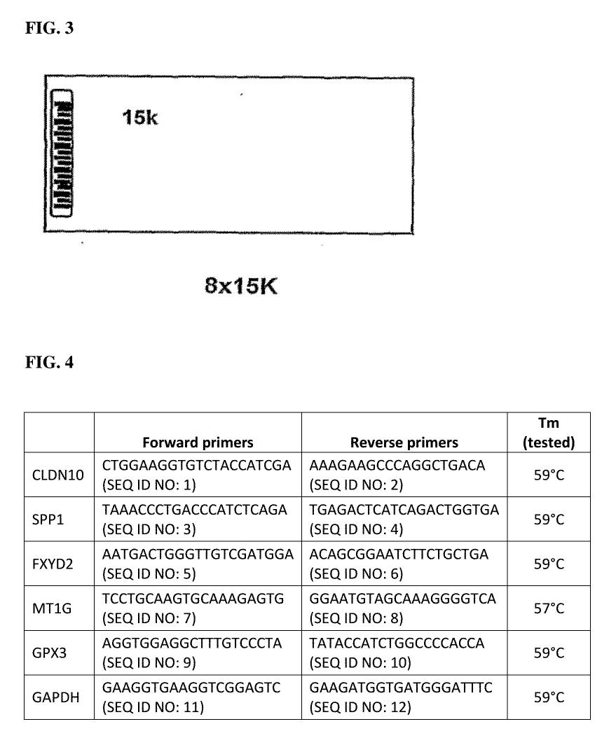

The forward and reverse primers were designed for five genes with increased LH+7 (FIG. 4). The oligonucleotide sequences of the primers were designed with the GeneFisher bioinformatic program (see FIG. 4 and sequence listing). The detection system was performed with SYBR Green I binding to double-stranded DNA (Roche). This detection system establishes a linear dynamic range for detecting specific PCR products. All the Q-PCR experiments were conducted using the SYBR Green PCR Master Mix (Roche) and the universal conditions of the thermal cycle parameters indicated by the manufacturers using the Roche Light Cycler. 40 cycles were performed. The temperatures at which the primers work well can be observed in FIG. 4. The relative quantification was performed by means of the standard master curve method.

The expression of GPX3; CLDN10; FXYD2; SPP1; and MT1G, correspond in the ERA to the expression values of the following probes:

| Probe | Gene |
|---|---|
| A_23_P133474 | GPX3 |
| A_23_P133475 | GPX3 |
| A_01_P007324 | CLDN10 |
| A_23_P48350 | CLDN10 |
| A_24_P196562 | FXYD2 |
| A_23_P161769 | EXYD2 |
| A_23_P7313 | SPP1 |
| A_01_P017618 | SPP1 |
| A_23_P60933 | MT1G |
| A_23_P206707 | MT1G |
| A_23_P206701 | MT1G |

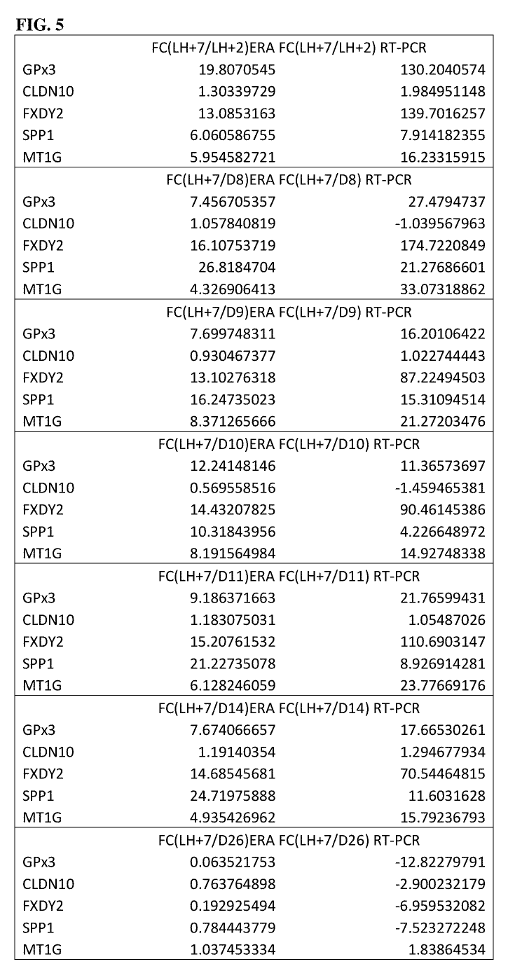

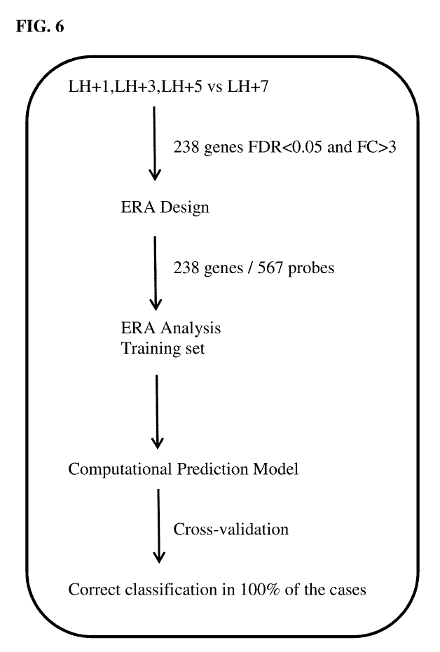

Considering that these are different techniques, quantitative PCR, the sensitivity of which is much higher but it only provides one expression value, and the arrays in the which there is expression of different probes for one and the same gene, in order to make the comparison, the mean expression of the different probes of a gene has been calculated in the array (FIG. 5).

Due to the different sensitivity, it is considered that the ratio of the expression value between both techniques would correspond to a correction factor of 10 (augmented expression 10× in the array) it is accepted that they correspond with a maximum of 100× in the quantitative PCR (FIG. 5).
3. Analyzing the expression pattern of the EPA during the window of implantation to be able to establish the endometrial receptivity profile. Generating a classifier.

### Training

A predictor is a mathematical tool which uses a data matrix, in this case of the data generated with the ERA, and learns to distinguish classes (Medina I, et al., 2007), in this case two or more classes according to the different receptivity profiles that are generated (normal receptive; pathological receptive; normal non-receptive . . . ). The underlying reasoning for this strategy is the following: if it is possible to distinguish among the classes as a consequence of the level of gene expression, it is then in theory possible to find the characteristic gene expression of LH+7 and to use it to assign a class to the expression profile of the test sample analyzed with the customized ERA microarray.

The set of samples which trains the classifier to define the classes is referred to as training set. In other words, the gene expression profiles of these samples, measured with the ERA, are used by the program to know which probes are the most informative and to distinguish between classes (different normal non-receptive and receptivity states). The biopsies used to generate the classification model are carefully chosen and dated in the most reliable manner currently available. This training set will gradually grow as a larger number of samples are tested, but it is made up of receptive samples and on other days of the menstrual cycle. They are all independent samples from different healthy women in the natural cycle and with proven fertility. They are Caucasian women with a body mass index between 19 and 25 kg/m² and between 19 and 34 years old. Only those samples the histological dating of which, by applying Noyes criteria, coincides between the two pathologists and with the day of the menstrual cycle have been chosen.

The classification is done by the bioinformatic program using different mathematical algorithms, there being many available. An algorithm is a well defined, ordered and finite list of operations which allows solving a problem. A final state is reached through successive and well-defined steps given an initial state and an input, obtaining a solution.

The classifier calculates the error committed by means of a process called cross-validation, which consists of leaving a subset of the samples of the training set of a known actual class out of the group for defining the classes, and then testing them with the generated model and seeing if it is right. This is done by making all the possible combinations. The efficacy of the classifier is calculated and prediction models are obtained which correctly classify all the samples of the training set (FIG. 5). In other words, all the samples of the training set are classified by the predictor in the assigned actual class known by the inventors.

A priori, it is impossible to know how the data are distributed in space, it is only possible to know how they are located in the dimensions that can be distinguished, there being three of them. Therefore, there are different algorithms to be applied which would work better or worse depending on how the entered data are distributed in space. The algorithms most widely used in mathematics for expression matrices generated by microarray analysis are applied, and the one that best separates the defined classes is observed. Therefore, there are algorithms which establish a separation according to a straight line, others do so depending on the closest nearby point, based on distances . . . and thus each method is based on a mathematical separation criterion which will more or less fit the reality of the samples.
4. Developing a predictor which allows quantitatively and objectively evaluating and predicting the endometrial receptive state based on the gene expression profile.

### Determining the Prediction

Depending on all the parameters relating to a computational predictor explained above, a prediction model is generated which classifies all the samples according to the assigned actual class, which in turn was dated by Noyes, there being a 100% coincidence (FIG. 7).

The generated prediction model has been trained with a training set of 23 samples, 12 receptive samples and 11 on other days of the menstrual cycle, two classes (receptive/Other) being distinguished. After that, the model will be re-trained as more samples of the same characteristics of the already generated training set are obtained, but also with samples in a receptivity period with pathologies altering the expression pattern of the ERA, as well as the alteration by drugs. Increasingly more classes will thus be gradually defined.

Therefore, the ERA can be used for the positive identification of the endometrial receptivity, as well as for the diagnosis of the alteration thereof associated with endometrial alterations typical of pathologies such as endometriosis, implantation failure, hydrosalpinx, etc. This diagnostic tool would also allow detecting functional modifications induced by interceptive drugs or drugs which intend to improve endometrial receptivity, altering the normalcy/abnormality situation in the receptive profile of the endometrium of a woman.

Therefore, the ERA of the present invention is a customized gene expression microarray. It is a 60-mer oligo array with 8 arrays per slide, with 15K (15744 points) in each array.

It is a customized array with design number 016088 (AMADID). It has 569 probes represented by 238 genes with 8 replicas for each probe, for a total of 4,536 points, 10,672 of which are free points. Reading the expression profile of the expression data for 238 genes represented by 569 probes (genes with an FDR>0.05 and an FC>3) is a prediction model constructed with 23 samples classified with an error of 0, which is capable of classifying the sample as receptive state or other.

The statistical analyses as well as the selection of genes with the indicated characteristics were done using computer programs.

The final list of the ERA includes the 569 probes representing the 238 genes with an FDR<0.05 and an FC>3 (FIG. 1).

The customized ERA array is hybridized with the messenger RNA of another set of samples different from those used to select the genes to be included, which are used to teach the predictor how to classify between LH+7 or another.

After defining these two classes, receptive or outside, the predictor will be scaled, i.e., it will determine how close or far the profile of a sample is from the receptive profile.

### EXAMPLE

### Obtaining and Processing the Samples

Biopsies of the endometrium were taken in 30 healthy female donors with proven fertility, and from 10 patients in a clinic with implantation failure due to an endometrial cause, the 4^{th} loopsies being taken on day 21 of the menstrual cycle (receptive phase, LH+7).

The total RNA of each of the biopsies is extracted using the Trizol protocol (Invitrogen) following the manufacturer's instructions (Life Technologies, Inc., USA). The samples are homogenized using 1 ml of Trizol for each 75 mg of tissue, they are incubated at room temperature for 5 minutes, and 200 μl of chloroform are added for the same amount of tissue and are incubated at room temperature for 5 minutes. They are then centrifuged for 15 minutes at 12,000×g (4° C.). The aqueous phase is precipitated with an equal volume of 2-propanol (isopropanol), it is incubated on ice for 5 minutes and centrifuged for 30 minutes at 12,000×g (4° C.). The precipitate is washed with 70% ethanol in water treated with diethylpyrocarbonate (DEPC) to subsequently resuspend it in DEPC-treated water (15 μl). This protocol usually produces 1-2 μg of total RNA per mg of endometrial tissue. The RNA thus extracted is treated with DNase for 1 hour at 37° C. to remove the traces of DNA and purify it again using the Qiagen RNeasy kit following the manufacturer's instructions. The RNA that is obtained after the columns of the RNeasy kit is analyzed to check its quality in the Agilent 2100 bioanalyzer using the Agilent brand RNA specific chips, RNA Nano LabChip.

Only those RNAs having the following characteristics can be used:

they did not have detectable genomic DNA,

they had a concentration greater than 200 μg/ml,

the value of the radius of rRNA was 28s/18S>1.2, and

the RIN (RNA Integrity Number) value>7.0.

After the analyses with the samples selected due to their suitable quality, single-stranded complementary DNA (cDNA) is generated from the total RNA by incubating it between one and two hours at 40° C. with reverse transcriptase, nucleotides and an oligonucleotide polydT-T7, which has not only the poly T sequence which hybridizes with the polyA tail of messenger RNA, but also the recognition sequence for T7 RNA polymerase.

The cDNA obtained in the previous step is incubated for 2 hours at 40° C. in the presence of T7 RNA polymerase and nucleotides, one of which is labeled with Cy3, to produce complementary RNA called cRNA.

That cRNA is purified by means of a purification kit based on affinity chromatography and is quantified.

Once purified, that labeled cRNA is fragmented for 30 minutes at 60° C. and hybridized in the microarray for 17 hours at 65° C. Once that time has elapsed, the microarray is washed to remove unspecific hybridizations. Once hybridized and washed, the microarrays are centrifuged at 3,000 rpm for 3 minutes to dry the microarrays and they are then read by means of scanning them in an Axon GenePix 4100A, reading for Cy3 intensities (532 nm).

As a result, after the relevant data processing enclosed below, a gene expression matrix is generated the rows of which correspond to the 569 probes of the 238 genes selected and the columns of which correspond to the different samples.

### Processed of the Data of the Array

The data of the array is processed by a series of bioinformatic commands which are in software designed exclusively for the invention as is explained below.

The correction of the bottom effect in the 40 data matrices due to the labeling process typical of the technique is performed.

The empty points are then removed and the normalization process is performed depending on the 40 samples and depending on the expression profile defined according to the prediction model so that it can be compared.

The mean of the eight replicas of each probe is then calculated. The different probes of the same gene are analyzed individually and the results are analyzed by the computational created prediction model which is also included in the software.

### Prediction

The 40 samples to be tested (test set) are run with the created classification model which analyzes the expression of the ERA and predicts which class they belong to.

### Results

The analysis of the expression data of the array was entered in the software. The obtained result indicated that out of the 30 tested samples from healthy women with proven fertility, 27 corresponded to women with an expression receptivity profile of the endometrium considered as normal and 3 corresponding to women with an expression receptivity profile of the endometrium considered as outside of normalcy. Nine out of the 10 patients with implantation failure were classified as outside of normal receptivity and 1 was classified as within normal receptivity. The molecular tool presented a 90% diagnostic efficacy.

### LITERATURE

Al Shahrour F and Dopazo J. In Azuaje F and Dopazo J (eds), Data analysis and visualization in genomics and proteomics. Wiley 2005; 99-112.

Al-Shahrour F, Minguez P, Vaquerizas J M, Conde L and Dopazo J. BABELOMICS: a suite of web tools for functional annotation and analysis of groups of genes in high-throughput experiments. Nucleic Acids Res 2005; 33:460-464.

Balasch J, Fabregues F, Creus M and Vanrell J A. The usefulness of endometrial biopsy for luteal phase evaluation in infertility. Hum Reprod 1992; 7:973-977.

Batista M C, Cartledge T P, Merino M J, Axiotis C, Platia M P, Merriam C R. Midluteal phase endometrial biopsy does not accurately predict luteal function. Fertil Steril 1993; 59:294-300.

Borthwick J, Charnock-Jones S, Tom B D et al, (2003) Determination of the transcript profile of human endometrium. Mol Hum Reprod 9, 19-33.

Carson D, Lagow E, Thathiah A et al, (2002) Changes in gene expression during the early to mid-luteal (receptive phase) transition in human endometrium detected by high-density microarray screening. Mal Hum Reprod 8, 971-979.

Catalano R D, Yanaihara A, Evans A L, Rocha D, Prentice A, Saidi S, Print C G, Charnock-Jones D S, Sharkey A M and Smith S K (2003) The effect of RU486 on the gene expression profile in an endometrial explant model Mol. Human. Reprod 9,465-473.

Coutifaris C, Myers E R, Guzick D S, Diamond M P, Carson S A, Legro R S, McGovern P G, Schlaff W D, Carr B R, Steinkampf M P, Silva S, Vogel D L and Leppert P C. Histological dating of timed endometrial biopsy tissue is not related to fertility status. Fertil Steril 2004; 82:1264-72.

Creus M, Ordi j, Fabregues F, Casamitjana R, Ferrer B, Coll E, Vanrell J A and Balasch J. Alphavbeta 3 integrin expression and pinopod formation in normal and out-of-phase endometria of fertile and infertile women. Hum Reprod 2002; 17:2279-2286.

Horcaladas J A, Sharkey A M, Catalano R D, Sherwin J R A, Dominguez F, Burgos L A, Castro A, Peraza M R, Pellicer A and Simön C (2006) Use of Gene-Expression Profiling to Identify Human Endometrial Refractoriness. J Clin Endocrinol Metabol.

Horcajadas J A, Pellicer A and Simón C (2007) Wide Genomic Analysis of Human Endometrial Receptivity. New times, new opportunities. Human Reprod Update 13, 77-86.

Horcajadas J A, Riesewijk A, Polman J, van Os R, Pellicer A, Mosselman S and Simón, C (2005) Effect of Controlled Ovarian Hyperstimulation in IVF on Endometrial Gene Expression Profiles. Mol Human Reprod 11,195-205.

Kliman H J, Honig S, Walls C, Luna M, McSweet J C, Copperman A B. Optimization of endometrial preparation results in a normal endometrial function test (EFT) and good reproductive outcome in donor ovum recipients. J Assist Reprod Genet 2006; 23:299-303.

Lessey B A, Castelbaum A J, Sawin S W, Sun J. Integrins as markers of uterine receptivity in women. with primary unexplained infertility. Fertil Steril 1995; 63:535-542.

Li T C, Dockery P, Rogers A W and Cooke I D. (How precise is histologic dating of endometrium using the standard dating criteria?. Fertil Steril 1989; 51:759-763.

Medina I, Montaner D, Tarraga J, Dopazo J. Prophet, a web-based tool for class prediction using microarray data. Bioinformatics. 2007; 23(3):390-1.

Mirkin S, Arslan M, Churikov D, Oorica A, Diaz J I, Williams S, Bocca S and Oehninger S (2005) In search of candidate genes critically expressed in the human endometrium during the window of implantation Human Reprod 20:2104-2117.

Mirkin S, Nikas C, Hsiu J G, Diaz J and Oehninger S (2004) Gene expression profiles and structural/functional features of the peri-implantation endometrium in natural and gonadotropin-stimulated cycles. J Clin Endocrinol Metab 89:5742-5752.

Montaner D, Tárraga J, Huerta-Cepas J, Burguet J, Vaquerizas J M Conde L, Minguez P, Vera. J, Mukherjee S, Valls J, Pujana M A G, Alloza E, Herrero J, Al-Shahrour F and Dopazo J. Next station in microarray data analysis: GEPAS. Accepted Nucleic Acids Res. 2006.

Murray M J, Meyer W R, Zaino R J, Lessey B A, Novotny D B, Ireland K, Zeng D and Fritz M A. A critical analysis of the accuracy, reproducibility, and clinical utility of histologic endometrial dating in fertile women. Fertil Steril 2004; 81:1333-1343.

Noyes R W, Hertig A T, and Rock J. Dating the endometrial biopsy. Fertil Steril 1950; 1:3-17.

Ordi J, Creus M, Quinto L, Casamitjana R, Cardesa A and Balasch J. Within-subject between-cycle variability of histological dating, alpha v beta 3 integrin expression, and pinopod formation in the human endometrium. J Clin Endocrinol Metab 2003; 88:2119-2125.

Papanikolaou E G, TouARNye H, Verpoest W, Camus M, VeARNeve V, Van Steirteghem A, Devroey P; http://www.ncbi.nlm.nih.govisites/entrez?Db=pubmed&Cmd=ShowDetailView&TermToSearch=15576388&ordinalpos=79&itool=EntrezSystem 2.PEntrez.Pubmed.Pubmed_ResultsPanel.Pubmed_RVDocSum Early and late ovarian hyperstimulation syndrome: early pregnancy outcome and profile. Hum Reprod. 2005; 20(3):'636-641.

Ponnampalam A P, Weston G C, Trajstman A C. Molecular classification of human endometrial cycle stages by transcriptional profiling. Mol Hum Reprod 2004; 10, 879-893.

Riesewijk A, Martin J, Horcajadas J A Polman J, Pellicer A, Mosselman S and Simón C (2003) Gene expression profiling of human endometrial receptivity on days LH+2 contra LH+7 by microarray technology. Mol. Hum Reprod 9:253-264.

Schena M, Shalon D, Davis R W and Brown P G. Quantitative monitoring of gene expression patterns with a complementary DNA microarray. Science 1995; 270:467-470.

Sharkey A M, Catalano R, Evans A, Charnock-Jones D S and Smith S K (2005) Novel antiangiogenic agents for use in contraception. Contraception 71,263-271.

Shoupe D, Mishell D R Jr, Lacarra M, Lobo R A, Horenstein J, d'Ablaing G. Correlation of endometrial maturation with four methods of estimating day of ovulation. Obstet Gynecol. Obstet Gynecol 1989; 73:88-92.

Talbi S, Hamilton A E, Vo K C, Tulac S, Overgaard M T, Dosiou C, Le Shay N, Nezhat, C N, Kempson R, Lessey B A, Nayak N B and Giudice L C. Molecular phenotyping of human endometrium distinguishes menstrual cycle phases and underlying biological processes in normb-ovulatory women. Endocrinology 2005; 147:1097-1121.

Wilcox A J, Baird D D, Weinberg C R. Time of implantation of the conceptus and loss of pregnancy. N Engl J Med. 1999; 340:1796-1799.

T-REX (http://www.gepas.org/)

FATIGO (http://babelomics.bioinfo.cipf.es/EntryPoint?load Form=fatigo)

PROPHET (http://qepas.bioinfo.cipf.es/cgi-bin/loadtool. cgi?tool=prophet) Agilent earray 4.5 (https://earray.chem.agilent.com/earray/)

1. A method for detecting in a biological sample obtained from human endometrium the normalcy/abnormality situation in the receptive profile of said endometrium, characterized in that it comprises:
a) obtaining an endometrial biopsy of the fundus of the uterus of a woman 7 days after her endogenous LH surge, which is equivalent to the phase of day 20-21 of the menstrual cycle.
b) performing the extraction and purification of mRNA of the endometrial biopsy;
c) determining in said sample the expression profile of the set of the genes involved in endometrial receptivity according to FIG. 1, included in the ERA by means of microarray technology;
d) detecting in said biopsy the expression profile of said genes involved in endometrial receptivity; and
e) analyzing said expression profile of the genes by means of the computer software containing the specific prediction model which classifies and determines the state of the endometrium depending on the gene profile with the established criteria.

2. The method according to claim 1, characterized in that the endometrial sample obtained in (a) is contacted with an oligo (probe) which is complementary to a region of the gene the expression of which is quantified.

3. The method according to claim 1, characterized in that each gene has at least one probe.

4. The method according to claim 1, characterized in that said mRNA is normally induced or suppressed in that phase of the cycle in a receptive endometrium and is suppressed or induced in that same phase of the feminine cycle in a nonreceptive endometrium.

5. The method according to claim 1, characterized in that the expression profile fits the one established by the prediction model for the ERA once the expression pattern of the ERA has been established during the window of implantation.

6. The method according to claim 1, characterized in that the situation of abnormality is caused by subfertility situations or due to an endometrial cause, such as the failure of the implantation or hydrosalpinx.

7. The method according to claim 1, characterized in that the normalcy/abnormality situation in the receptivity profile of the endometrium is due to the effect of drugs or inert devices, or in combination with drugs which alter the normalcy/abnormality situation.

8. The method according to claim 7 for detecting in a biological sample the effect of drugs for detecting which alter the normalcy/abnormality situation in the receptive profile of an endometrium, characterized by the expression profile determined by the ERA, both in a healthy woman and in a woman with various pathological conditions affecting the gene expression profile of the genes included in the ERA, such as implantation failure due to an endometrial cause, for example.

9. The method according to claim 1, characterized in that the receptivity marker used consists of determining the joint expression profile of the genes according to FIG. 1.

10. A microarray for carrying out the method according to claim 1.

11. The microarray according to claim 10, characterized in that it is a customized expression microarray formed by oligos (probes) detecting the mRNA set of the sample.

12. The microarray according to claim 10, characterized in that it defines and classifies the expression receptivity profile by means of a computational prediction model.

13. The microarray according to claim 10, characterized in that it defines the normal receptivity state and of other receptivity situations of both subfertility and infertility, as well as exposure to drugs.

14. The microarray according to claim 10, characterized in that it uses software containing all the information necessary for an endometrial biopsy taken during the receptive period to be classified in the class determined by the prediction model after being analyzed by the ERA and after its gene expression data are preprocessed.

15. The microarray according to claim 10, characterized by having a format of 8×15K (8 arrays of 15,000 probes) per slide.

16. The microarray according to claim 10, characterized in that it comprises the 569 probes of FIG. 1 represented by the 238 genes of FIG. 2.

17. The microarray according to claim 10, characterized in that with the constructed prediction model it is capable of classifying the normalcy/abnormality situation in the receptive profile of said sample.

18. A kit comprising:
a) the microarray according to claim 10, and
b) instructions for use.

19. A kit comprising:
a) the microarray according to claim 10,
b) software which processes, analyzes and predicts from the microarray data, and
c) instructions for use.
